(19) United States
(12) Patent Application Publication
(10) Pub. No.: US 2025/0161668 A1
(43) Pub. Date: May 22, 2025

(54) METHODS AND DEVICES FOR ELECTRODE IMPLANTATION

(71) Applicant: Amber Therapeutics Ltd, London (GB)

(72) Inventors: Charles Knowles, London (GB); Stefan De Wachter, Antwerp (BE)

(21) Appl. No.: 18/841,150

(22) PCT Filed: Feb. 22, 2023
(86) PCT No.: PCT/IB2023/000106

§ 371 Date: Aug. 23, 2024

Related U.S. Application Data
(60) Provisional application No. 63/313,437, filed on Feb. 24, 2022.

(51) Int. Cl. A61N 1/05

(52) U.S. Cl. CPC A61N 1/0558

(57) ABSTRACT

Provided herein are devices, systems, and methods for accessing the pudendal nerve to place an electrical lead on the pudendal nerve for treatment by electrical nerve stimulation. Described herein are devices, systems, and methods for providing electrical nerve stimulation to prevent an episode of incontinence in an individual in need thereof.

### CROSS-REFERENCE

This application claims the benefit of U.S. Provisional Application No. 63/313,437 filed Feb. 24, 2022 which is incorporated herein by reference in its entirety.

### BACKGROUND

Incontinence, or a lack of control over micturition or bowel movements, has many causes but commonly involves injury, weakness, or overactivity of the pelvic floor muscles and the nerves that innervate these muscles and involved organs. Electrical stimulation has been used to treat incontinence by stimulating the muscle directly or the sacral nerve to improve control over micturition and defecation. Approaches to stimulate other nerves or structures to improve incontinence may be limited by the ability to access these nerves and structures.

### SUMMARY

A lack of voluntary control over micturition, defecation, incontinence, or any combination thereof is a problem that can impact quality of life and cause social embarrassment. Urinary incontinence, or loss of bladder control, and fecal incontinence, loss of control of bowel movements, often relate to neurological issues.

To treat or reduce symptoms of incontinence, electrical stimulation of the muscle, sacral nerve, and/or other pelvic nerves involved in incontinence has been used by to improve control over micturition and bowel movements. In some cases, current electrical stimulation therapies may include sacral neuromodulation (SNM). While SNM may reduce the frequency of incontinence episodes, the success of SNM may be limited. SNM may provide a complete cure much less frequently.

As such, targeting another nerve, such as pudendal nerve, may provide an improved approach to treating incontinence. Often, the pudendal nerve contributes to motor functions and mediating volitional contraction of the urethral and anal sphincter muscles in the preservation of continence. In some cases, targeting of pudendal nerve may be combined with a closed-loop capability with dynamic adaptive control to provide a more effective treatment for incontinence. However, pudendal nerve has not been a target for neuromodulation to the same degree as the sacral nerve, partly due to the difficulty in accessing and fixing leads on or close to the pudendal nerve.

Provided herein are devices for placing an electrode lead to a target site, the device comprising: an introducer sheath comprising a sheath elongate shaft having a lumen and a sheath handle at a distal end of the sheath shaft, wherein the introducer sheath is configured to carry at least one electrode on an outer surface of the sheath elongate shaft; an obturator comprising an obturator elongate shaft having a lumen and an obturator handle at a distal end of the obturator elongate shaft, wherein the obturator elongate shaft is configured to fit inside the sheath lumen; and a needle comprising a needle elongate shaft having a lumen, a needle handle at a distal end of the needle elongate shaft, and a needle tip at the proximal end of the needle elongate shaft, wherein the needle elongate shaft is configured to fit inside the obturator lumen, and wherein the lumen of the needle elongate shaft is configured to allow the electrode lead to pass through: wherein the introducer sheath has a stiffness sufficient to guide the placement of the electrode lead at the target site, wherein the target site is a pudendal nerve. In some embodiments, the obturator handle comprise a latch configured to attach to the sheath handle. In some embodiments, the at least one electrode comprises a flexible printed circuit. In some embodiments, the at least one electrode is wrapped around the outer surface of the sheath elongate shaft. In some embodiments, the at least one electrode is bonded to the outer surface of the sheath elongate shaft. In some embodiments, the needle tip is blunt and has a lumen. In some embodiments, the needle tip is configured to protrude at least 1 mm beyond the end of the obturator lumen. In some embodiments, the needle tip is configured to protrude by a movement of the needle handle. In some embodiments, the needle tip is configured to be retractable into the obturator lumen. In some embodiments, the needle tip has an angle ranging from about 15 degrees to about 45 degrees from the needle elongate shaft. In some embodiments, the needle tip angle is configured to advance the needle tip through tissue. In some embodiments, a proximal end of the sheath elongate shaft is angled to form an angle of about 30 degrees to about 90 degrees. In some embodiments, the angle of the proximal end of the sheath elongate shaft allows for advancing the device with little to no damage to surrounding tissue. In some embodiments, the needle has a diameter of about 0.4 mm to about 2 mm. In some embodiments, the needle has a diameter of between 12 and 26 gauge. In some embodiments, the obturator has a diameter of about 1 mm to about 4 mm. In some embodiments, the introducer sheath has a diameter of about 1 mm to about 5 mm. In some embodiments, the sheath elongate shaft comprises high density polyethylene (HDPE). In some embodiments, the obturator elongate shaft comprises stainless steel. In some embodiments, the needle elongate shaft comprises stainless steel. In some embodiments, the introducer sheath has a Young's modulus of about 10 mega pascal (MPa) to about 10,000 MPa. In some embodiments, the sheath elongate shaft has a Young's modulus of about 10 mega pascal (MPa) to about 10,000 MPa. In some embodiments, the introducer sheath and the needle have a combined Young's modulus sufficient to allow for the device to penetrate a deep surgical plane in an individual. In some embodiments, the deep surgical plane comprises a surgical plane of muscle, fat, or any combination thereof. In some embodiments, the introducer sheath and needle have a combined Young's modulus to sufficient allows a user to place the lead adjacent to the pudendal nerve.

Described herein are methods for accessing a pudendal nerve in a subject, the method comprising: (a) creating an opening on a skin surface medial to ischial tuberosity; (b) advancing an electrode introducer through the opening to a predetermined depth; (c) deploying and advancing a needle tip of the electrode introducer to pass close to or penetrate through a ligamentous tissue; and (d) accessing the pudendal nerve. In some embodiments, the method further comprises placing at least one electrode to the pudendal nerve, wherein the electrode introducer is configured to hold and deliver the electrode. In some embodiments, the ligamentous tissue comprises a sacrotuberous ligament. In some embodiments, at least one electrode is placed at the pudendal nerve trunk proximal to Alcock's canal. In some embodiments, the method further comprises identifying and providing on a skin surface a plurality of surface markings corresponding a plurality of anatomical locations based an image of the plurality of anatomical locations, wherein the plurality of surface markings is used to determine the location of the opening on the skin surface. In some embodiments, the plurality of anatomical locations comprises one or more of medial border of ischial bone, lateral border of ischial bone, superior border of inferior pubic ramus, inferior border of inferior pubic ramus, inferior border of acetabulum, and superior border of greater trochanter of femur. In some embodiments, the image is obtained by fluoroscopy. In some embodiments, the plurality of surface markings comprises one or more of a horizontal line through the superior tip of the greater trochanter of the femur (L3), a horizontal line through the inferior border of the acetabulum (L2), a curved line on the inner surface of the ischial bone (L1), and a marking representing the ischial spine that crosses with L3 line. In some embodiments, the pudendal nerve trunk is located in an area medial to the L1 curved line and substantially superior to the L2 line. In some embodiments, a distal portion of the pudendal nerve is located in an area medial to the L1 curved line and substantially inferior to L2 line. In some embodiments, an electrode needle is used to determine presence of electrophysiological responses before proceeding with introducer insertion. In some embodiments, an electrode needle is used to determine laterality of desired electrophysiological responses before proceeding with introducer insertion. In some embodiments, the desired electrophysiological response comprises a detectable EMG response at a stimulation amplitude at less than about 4 milliampere. In some embodiments, the electrode introducer is advanced toward the ischial spine. In some embodiments, the method further comprises retracting the needle tip into the electrode introducer prior to placing the at least one electrode at the pudendal nerve. In some embodiments, advancing the electrode introducer is guided by imaging. In some embodiments, the imaging comprises fluoroscopy. In some embodiments, the method further comprises stimulating at least one electrode on the pudendal nerve at a low stimulation amplitude. In some embodiments, the method further comprises observing contraction of the anal sphincter or a urethral pressure contraction or a combination thereof. In some embodiments, an electromyography (EMG) reading of the contraction of the anal sphincter is obtained from at least three contact points. In some embodiments, the method further comprises obtaining an electromyography (EMG) reading of a pelvic floor muscle. In some embodiments, the method further comprises obtaining a transvaginal electromyography (EMG) reading. In some embodiments, the EMG reading is taken from at least three contact points. In some embodiments, the placement of the at least one electrode is adjusted based on the EMG reading. In some embodiments, the method further comprises verifying the placement of the at least one electrode by electrical stimulation. In some embodiments, the method further comprises verifying the placement of the at least one electrode by EMG of the anal sphincter and the pelvic floor. In some embodiments, the method further comprises verifying the placement of the at least one electrode by X-ray imaging. In some embodiments, the method further comprises removing the electrode introducer through the opening after placing the at least one electrode at the pudendal nerve. In some embodiments, the subject has experienced or is at risk for experiencing an episode of incontinence.

Provided herein are methods for accessing a pudendal nerve in a subject, the method comprising: (a) creating an opening on a skin surface at a predetermined incision distance laterally from inner brim of ischial bone; (b) advancing an electrode introducer through the opening down to the ischial bone and along the medial border of the ischial bone; (c) advancing the electrode introducer following a curve of the inferior pubic ramus at or above inferior border of obturator foramen and in the direction of the pubic symphysis; and (d) accessing the pudendal nerve. In some embodiments, the method further comprises placing at least one electrode at the pudendal nerve, wherein the electrode introducer is configured to hold and deliver the at least one electrode. In some embodiments, the at least one electrode is placed at an anterior portion of the pudendal nerve or branches thereof. In some embodiments, the anterior branch of the pudendal nerve comprises the dorsal genital nerve. In some embodiments, the method further comprises advancing the electrode introducer anteriorly in the ischiorectal fossa and below the pelvic floor in step (c). In some embodiments, the method further comprises identifying and providing on a skin surface a plurality of surface markings corresponding a plurality of anatomical locations based an image of the plurality of anatomical locations, wherein the plurality of surface markings is used to determine the location of the opening on the skin surface. In some embodiments, the plurality of anatomical locations comprises one or more of medial border of ischial bone, lateral border of ischial bone, superior border of inferior pubic ramus, inferior border of inferior pubic ramus, inferior border of acetabulum, and superior border of greater trochanter of femur. In some embodiments, the image is obtained by fluoroscopy. In some embodiments, the plurality of surface markings comprises one or more of a horizontal line through the superior tip of the greater trochanter of the femur (L3), a horizontal line through the inferior border of the acetabulum (L2), a curved line on the inner surface of the ischial bone (L1), and a marking representing the ischial spine that crosses with L3 line. In some embodiments, the pudendal nerve trunk is located in an area medial to the L1 curved line and substantially superior to the L2 line. In some embodiments, a distal portion of the pudendal nerve is located in an area medial to the L1 curved line and substantially inferior to L2 line. In some embodiments, advancing the electrode introducer is guided by imaging. In some embodiments, the imaging comprises fluoroscopy. In some embodiments, the method further comprises stimulating the at least one electrode on the pudendal nerve at a low stimulation amplitude. In some embodiments, the method further comprises observing contraction of the anal sphincter. In some embodiments, an electromyography (EMG) reading of the contraction of the urethral or anal sphincter is taken at least one contact point. In some embodiments, the method further comprises obtaining an electromyography (EMG) reading of a pelvic floor muscle. In some embodiments, the method further comprises obtaining a transvaginal electromyography (EMG) reading. In some embodiments, the EMG reading is taken at least one contact point. In some embodiments, the method further comprises verifying the placement of the at least one electrode by electrical stimulation. In some embodiments, the method further comprises verifying the placement of the at least one electrode by EMG of the anal sphincter and the pelvic floor. In some embodiments, the method further comprises verifying the placement of the at least one electrode by X-ray imaging. In some embodiments, the method further comprises removing the electrode introducer through the opening after placing the at least one electrode at the pudendal nerve. In some embodiments, the subject has experienced or is at risk for experiencing an episode of incontinence.

Provided herein are methods for securing an electrode lead to a tissue near a pudendal nerve, the method comprising: (a) creating an opening on a skin surface near a pudendal nerve; (b) advancing an over-sheath releasably holding an electrode lead and an anchor through the opening to a target location at or near the pudendal nerve, wherein the anchor comprises one or more securing arms on an outer surface of the anchor, wherein the anchor securely holds the electrode lead near a distal end of the electrode lead; (c) releasing the over-sheath from the electrode lead and the anchor, wherein the anchor is exposed to a tissue at the target location and the one or more securing arms of the anchor is deployed into the tissue; and (d) securing the anchor and the electrode lead into the tissue. In some embodiments, the method further comprising removing the over-sheath from the opening. In some embodiments, creating the opening comprises dilatating a tissue around the opening. In some embodiments, the dilatation is achieved by a dissection tool. In some embodiments, the dilatation is achieved by the over-sheath. In some embodiments, the tissue is adjacent to ischial bone and fascial or ligamentous insertions. In some embodiments, the deployment of the one or more securing arms of the anchor into the tissue comprises the one or more securing arms expanding outwardly from the anchor. In some embodiments, the one or more securing arms are spring-loaded. In some embodiments, the anchor comprises a passive anchor. In some embodiments, the tissue to which the anchor and the electrode lead are secured comprises one or more of ligamentous, fascial, or periosteal tissues. In some embodiments, the securing further comprises suturing of the anchor to the tissue. In some embodiments, the anchor securely holds the electrode lead by a frictional force between the anchor and the electrode lead. In some embodiments, the anchor securely holds the electrode lead by ligature of the anchor to the electrode lead. In some embodiments, the ligature comprises compression of the anchor onto the electrode lead. In some embodiments, the ligature comprises using a screw. In some embodiments, the ligature comprises using a ligating clip. In some embodiments. the electrode lead comprises one or more distal lead tips. In some embodiments, the one or more distal lead tips are configured to be substantially straight when covered by an introducer and substantially curved when the introducer retracted from the distal tip. In some embodiments, the curve of the one or more distal lead tips aids in securing the electrode lead to the tissue.

Provided herein are systems for securing an electrode lead to a tissue near a pudendal nerve, the device comprising: (a) an electrode lead; (b) an anchor comprising one or more securing arms on an outer surface of the anchor, wherein the anchor is configured to hold the electrode lead; and (c) an over-sheath having a lumen, wherein an inner diameter of the lumen is sufficiently large to releasably hold the anchor and the electrode lead, wherein the one or more securing arms are configured to expand outward when the anchor is released from the over-sheath. In some embodiments, the tissue is adjacent to ischial bone and fascial or ligamentous insertions. In some embodiments, the tissue comprises one or more of ligamentous, fascial, or periosteal tissues. In some embodiments, the outward expansion of the one or more securing arms deploys the one or more securing arms into the tissue. In some embodiments, the one or more securing arms are spring-loaded. In some embodiments, the anchor comprises a passive anchor. In some embodiments, the electrode lead is secured to the tissue by suturing of the anchor holding the electrode lead to the tissue. In some embodiments, the anchor holds the electrode lead by a frictional force between the anchor and the electrode lead. In some embodiments, the anchor holds the electrode lead by ligature of the anchor to the electrode lead. In some embodiments, the ligature comprises compression of the anchor onto the electrode lead. In some embodiments, the ligature comprises using a screw.

The system of claim 28 or any preceding claims, wherein the ligature comprises using a ligating clip. In some embodiments, the electrode lead comprises one or more distal lead tips. In some embodiments, the one or more distal lead tips are configured to be substantially straight when covered by an introducer and substantially curved when an introducer is retracted from the distal tip. In some embodiments, the curve of the one or more distal lead tips aids in securing the electrode lead to the tissue.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1F show an exemplary embodiment of the electrode lead introducer.

FIGS. 2A-2F show an exemplary embodiment of the electrode lead introducer with electrode disks and insulating members.

FIGS. 3A-3D show an exemplary embodiment of the electrode lead introducer with planar electrodes.

FIGS. 4A-4E show an exemplary embodiment of the electrode lead introducer with coil electrodes.

FIGS. 5A-5C show an exemplary embodiment of the electrode lead introducer with coated electrodes.

FIGS. 6A-6D show an exemplary embodiment of the electrode lead introducer with printed electrodes.

FIG. 7 shows an exemplary flowchart of the steps in for performing electrode lead and implantable pulse generator (IPG) implantation.

FIG. 8 shows an exemplary schematic of the anatomy and the disposition of leads and IPG in an individual.

FIG. 9 shows an exemplary schematic of the anatomy and implanted leads and IPG in an individual.

FIG. 10 show exemplary schematics of the anatomy and implanted leads and IPG in an individual.

FIGS. 11A and 12B show exemplary embodiments of the anatomical paths of the leads using anatomical models.

FIGS. 12A-12G show exemplary embodiments of determining and making surface markings using fluoroscopy. FIGS. 12A-12F show fluoroscopy images with a straight metal instrument 1502.

FIGS. 13A-13B show exemplary embodiments of the markings on a radiological image (FIG. 13A) and on skin in the gluteal region (FIG. 13B).

FIGS. 14A-14B show exemplary embodiments of a fluoroscopy image and a schematic in the lateral view. The lateral depth from the level of the ischial spin to the surgical table may be determined to provide the A-P (anterior-posterior) depth of posterior surface of the ischial bone to guide insertion of the inserter.

FIG. 15 shows an exemplary embodiment of the lateral depth marking from X-ray.

FIGS. 16A-E show exemplary embodiments of sequential fluoroscopic images of insertion of the introducer and lead by ischiorectal approach toward the ischial spine in A-P view. The electrodes on the lead may be seen as four dots along distal end of the lead targeting the pudendal nerve.

FIGS. 17A-B show exemplary embodiments of sequential fluoroscopic images of insertion of the introducer and the lead after the introducer is removed by ischiorectal approach in lateral view, where the electrodes on the lead may be seen as four dots along the pudendal nerve.

FIG. 18 shows an exemplary embodiment of the insertion point for the low gluteal approach as indicated by the distal oval.

FIG. 19 shows an exemplary embodiment of fluoroscopic image of the insertion of the introducer by the low gluteal approach, where the introducer is directed along the line of the inferior border of the obturator foramen towards the pubic symphysis.

FIG. 20 shows an exemplary embodiment of a fluoroscopic image in the AP view of insertion of the lead by the low gluteal approach after the introducer has been removed and two leads with four electrodes on each lead (as indicated by four circles) are in place on the pudendal nerve.

FIGS. 21A and 21B: show an exemplary embodiment of a fluoroscopic images in anterior-posterior (AP) (FIG. 21A) and lateral (FIG. 21B) views where electrophysiology has determined a higher inflexion point of the pudendal nerve in the obturator foramen. In this embodiment, the leads can be seen to cross as the pudendal nerve turns forward in its course.

FIG. 22 shows an exemplary embodiment of a fluoroscopic image in the lateral view of insertion of the lead by the low gluteal approach and confirmation of position of both leads.

FIG. 23A show an exemplary embodiment of an anchor that has been slid onto the lead and a polyester stay suture taken through periosteum of ischium using a J needle. In some cases, the suture is tied around the groove of the passive anchor to compress the suture onto the lead.

FIG. 23B shows an exemplary embodiment of the anchor that is drawn into the tissue by tightening the stay suture after a second ligature is used on the second groove of the anchor.

FIGS. 24A-24D: shows and exemplary embodiment of a method for anchoring the electrode lead using a soft tissue tunnel (FIG. 24A and FIG. 24B) and deployment of a device as extruded from an over-sheath (FIGS. 24C-24D).

FIGS. 25A-25C show exemplary embodiments of a change in conformation of the electrode lead tip as the introducer is retracted.

FIG. 26 shows an exemplary embodiment of IPG pocket created in upper buttock.

FIGS. 27A and 27B show exemplary images of a sequence of tunnelling the leads, where FIG. 27A shows the ischiorectal lead taken through the low gluteal lead site and FIG. 27B shows both leads taken through to the IPG pocket.

FIG. 28 shows an exemplary image of the patient controller in a sterile bag to check impedance.

FIG. 29 shows a workflow for a method of securing an electrode lead to a tissue near a pudendal nerve.

### DETAILED DESCRIPTION

A lack of voluntary control over micturition, defecation, incontinence, or any combination thereof is a problem that can impact quality of life and cause social embarrassment. Urinary and fecal incontinence may affect individuals of all ages. Usually, older individuals may exhibit a greater probability of incontinence with varied pathophysiology Urinary incontinence, or loss of bladder control, and fecal incontinence, loss of control of bowel movements, often relate to neurological issues. Both urinary incontinence and fecal incontinence may involve injury, weakness, or overactivity of the pelvic floor muscles, including but not limited to the urethral and anal sphincter, and the nerves that innervate these muscles and involved organs, such as the bladder, rectum, or anus.

To treat or reduce symptoms of incontinence, electrical stimulation of the muscle, sacral nerve, and/or other pelvic nerves involved in incontinence has been used by to improve control over micturition and bowel movements. In some cases, current electrical stimulation therapies may include sacral neuromodulation (SNM) that may provide fixed patterns of stimulation to treat ‘urge’ (the sudden need to urinate), but such stimulation may be unable to respond to the more common ‘stress’ incontinent events, such as coughing, sneezing, and lifting. While SNM may reduce the frequency of incontinence episodes, the success of SNM may be limited in scope (e.g., not a cure) and may decrease over time. Furthermore, SNM may not be suitable to treat individuals having stress incontinence or mixed urinary incontinence (with stress and urge incontinence). In some cases, SNM may have high long-term costs in management of the electrical stimulation device and may require high level of skill and precision from the surgeon to place the lead on the sacral nerve. As such, targeting another nerve such as pudendal nerve, may provide an improved approach to treating incontinence.

Often, the pudendal nerve contributes to motor functions and mediating volitional contraction of the urethral and anal sphincter muscles in the preservation of continence. In some cases, targeting of pudendal nerve may be combined with a closed-loop capability with dynamic adaptive control to provide a more effective treatment for incontinence. In some cases, pudendal nerve stimulation (PNS) may provide a more effective treatment for individuals having incontinence than SNM. In some cases, PNS may be an effective treatment for incontinence in individuals where SNM has failed. In some cases, PNS may provide an effective treatment for urinary incontinence. In some cases, PNS may provide an effective treatment for chronic pain. In some cases, the pudendal nerve may be an effective continence target for closed loop stimulation.

However, the pudendal nerve has not been a target for neuromodulation to the same degree as the sacral nerve, despite the dominant role of the pudendal nerve in preservation of urinary continence. In some cases, the complex 3D anatomy of the pudendal nerve may make the pudendal nerve a difficult target for treatment by electrical stimulation. In some cases, the complex three-dimensional anatomy of the pudendal nerve may make accurate electrode placement more challenging. In some cases, electrophysiological guidance, rather than or in combination with radiological guidance, may be needed for accurate placement of electrodes on the pudendal nerve. In some cases, there may be uncertainty regarding which section of the pudendal nerve to target (e.g., trunk vs. branches) to provide an effective electrical stimulation treatment. In some cases, there may be difficulties with lead fixation due to the anatomy near the pudendal in comparison to the sacral nerve, which has a boney anatomy of sacrum to facilitate lead fixation. In some cases, there may be a concern that direct targeting of a peripheral nerve may be limited by issues of stimulation tolerability.

Provided herein are devices, systems, and methods for accessing the pudendal nerve to place an electrical lead on the pudendal nerve for treatment by electrical nerve stimulation. Described herein are devices, systems, and methods for providing electrical nerve stimulation to prevent an episode of incontinence in an individual in need thereof. In some cases, the devices, systems, and methods for placing an electrical lead to a target nerve site comprises an introducer sheath, an obturator, and a needle. In some cases, the introducer sheath comprises a sheath elongate shaft having a lumen, a sheath handle at a distal end of the elongated body, wherein the introducer sheath is configured to receive the electrode on an outer surface of the sheath elongate shaft. In some case, the obturator comprises an obturator elongate shaft having a lumen and an obturator handle at a distal end of the obturator elongate shaft, wherein the obturator elongate shaft is configured to fit inside the sheath lumen. In some cases, the needle comprises a needle elongate shaft, a needle handle at a distal end of the needle elongate shaft, and a needle tip at the proximal end of the needle elongate shaft, wherein the needle elongate shaft is configured to fit inside the obturator lumen. In some cases, the devices, systems, and methods for placing an electrical lead to a target nerve site described herein allows for easier access of the pudendal nerve and for an accurate electrode placement despite its complex three-dimensional anatomy. In some cases, the use of a closed loop algorithm stimulation on the pudendal nerve may reduce or minimize issues with issues of stimulation tolerability, which may reduce the effectiveness of PNS over time. In some cases, the devices, systems, and methods provided herein may be compatible with electrophysiological guidance, alone or in combination with radiological guidance, for accurate and reproducible placement of electrodes on the pudendal nerve. In some cases, the devices, systems, and methods provided herein allow for more accurate and reproducible placement of electrodes on the difficult-to-access pudendal nerve that vary less with the skill of the healthcare professional performing the procedure.

Described herein are devices, systems, and methods for accessing a pudendal nerve in a subject to place and fix an electrical lead on to the pudendal nerve. The devices, systems, and methods described herein may provide electrical nerve stimulation to prevent an episode of incontinence in an individual in need thereof. In some cases, the devices, systems, and methods provided herein may access the pudendal nerve by ischiorectal approach. In cases, the ischiorectal approach comprises where the lead introducer penetrates or passes close to the sacrotuberous ligament and directs the lead to the pudendal nerve trunk at a location proximal to Alcock's canal. In some cases, the devices, systems, and methods provided herein may access the pudendal nerve by a low gluteal approach, also referred herein as a low posterior approach. In some cases, the low gluteal approach comprises where the lead introducer and the lead passes in a space between sacrotuberous and sacrospinous ligaments and passes anteriorly in the ischiorectal fossa below the pelvic floor. In some cases, the lead placed using the low gluteal approach may stimulate the anterior branches of the pudendal nerve (including the dorsal genital nerve).

Often, devices, systems, and methods provided herein may address various shortcomings of SNM. In some embodiments, the devices, systems, and methods provided herein may provide higher rates of functional cure and better symptom control. In some embodiments, the devices, systems, and methods provided herein may provide a wider scope of patient access, including but not limited to patients having stress-related urinary incontinence (SUI), mixed urinary incontinence (MUI), and overactive bladder (OAB). In some embodiments, the devices, systems, and methods provided herein may provide a wider scope of patient access, including but not limited to patients having fecal incontinence. In some embodiments, the devices, systems, and methods provided herein may reduce the need for modification of an ongoing therapy and reduce the associated costs with such modifications. In some embodiments, the devices, systems, and methods to access the pudendal nerve may facilitate easy training and may be replicable to a high standard, resulting in high consistency and reproducibility in patient outcomes. In some embodiments, the electrical stimulation of the pudendal nerve may provide an ideal continence target for future closed loop electrical stimulation.

### Electrical Stimulation for Incontinence

Described herein are devices, systems, and methods to prevent an episode of incontinence in an individual in need thereof by providing electrical nerve stimulation. In some instances, the episode of incontinence may comprise urinary incontinence, fecal incontinence, or any combination thereof. The devices, systems and methods disclosed herein may treat a sub-type of incontinence. In some cases, the sub-type of incontinence may comprise urge incontinence, stress incontinence, overflow incontinence, or mixed incontinence.

Urinary incontinence may be categorized into one of four main types: urge incontinence, stress incontinence, overflow incontinence, and mixed incontinence. Urge incontinence is often due to an overactive bladder (OAB). Individuals with urge incontinence have a strong and sudden need to urinate immediately, often leaving them with insufficient time to reach a bathroom. Stress urinary incontinence (SUI) is usually due to a poorly functioning urethral sphincter muscle or hypermobility of the urethra or bladder neck. An individual may experience stress incontinence during activities such as coughing, sneezing, laughing, lifting, or exercise. Overflow incontinence may typically be due to poor bladder contraction or blockage of the urethra. Mixed urinary incontinence (MUI) may involve features of stress and urge incontinence. Incontinence often involves neurological issues, including but not limited to impaired nerve conduction between the brain and/or the affected muscles, and nervous system conditions or injuries (e.g., multiple sclerosis or stroke), or mental confusion. Other causes of incontinence include but are not limited to weakness of pelvic or urethral muscles and pelvic prolapse.

Fecal incontinence, also referred to as bowel incontinence, is the loss of bowel control, causing an individual to pass stool unexpectedly from the rectum. Fecal incontinence is usually categorized into three main types: urge incontinence, passive incontinence and post-defectory leakage (or a combination thereof). Individuals with urge incontinence have a strong and sudden need to defecate immediately, often leaving them with insufficient time to reach a bathroom. Passive fecal incontinence is when an individual passes feces without conscious awareness. Individuals suffering from passive incontinence cannot consciously control their bowel movements and stool can pass without their knowledge. Incontinence often involves neurological issues, including but not limited to impaired nerve conduction between the brain and/or the affected muscles, and nervous system conditions or injuries (e.g., multiple sclerosis or stroke), or mental confusion. Causes of fecal incontinence include but are not limited to nerve damage, anal sphincter muscle damage, constipation, diarrhea, surgery, loss of rectum storage capacity, rectal prolapse, and rectocele.

Sometimes, electrical stimulation of muscles has been used to treat incontinence by training the pelvic floor muscles thereby improving strength and function of the muscle to control over urination and defecation. In some case, electrical stimulation may target the sacral nerve to improve control over urination and defecation. In some case, the electrical stimulation approaches may benefit from stimulation of an alternate target.

In some cases, the electrical stimulation approaches may be capable of delivering only a predefined stimulation protocol and may not be able to adapt to the condition and circumstances of the individual during a particular episode of incontinence. In some cases, this may result in overstimulation or under stimulation of the target tissue, resulting in inadequate control over muscles involved in urination or bowel movements. Usually, traditional approaches to treating incontinence may not be able to mimic sufficiently an innate human response (i.e., reflex) to prevent an incontinence episode and may be insufficient. In some cases, individuals who have an incontinence episode may experience insufficient preventative response. In some cases, the preventative response may include a muscle contraction of at least one pelvic floor muscle to prevent a leakage event in response to an increased intra-abdominal pressure. In some cases, individuals who experience stress incontinence may exhibit a delayed response in preventing an incontinence episode in response to a stress event. In some cases, individuals may experience stress incontinence related to urethral hypermobility (i.e., insufficient support) that may lead to an increased pressure transmitted to the bladder and subsequently an incontinence event.

There is a need for devices, systems, and methods to deliver an electrical stimulation that accounts for the condition of the individual and adapts the stimulation level accordingly. In some cases, closed loop control may account for the condition of the individual and adapt the level of electrical stimulation. In some cases, the closed loop control may provide a ‘synthetic reflex’ that provides the individual control over incontinence at all times. In some case, the electrical stimulation approaches may benefit from stimulation of an alternate target such as the pudendal nerve.

### Electrode Lead Introducer

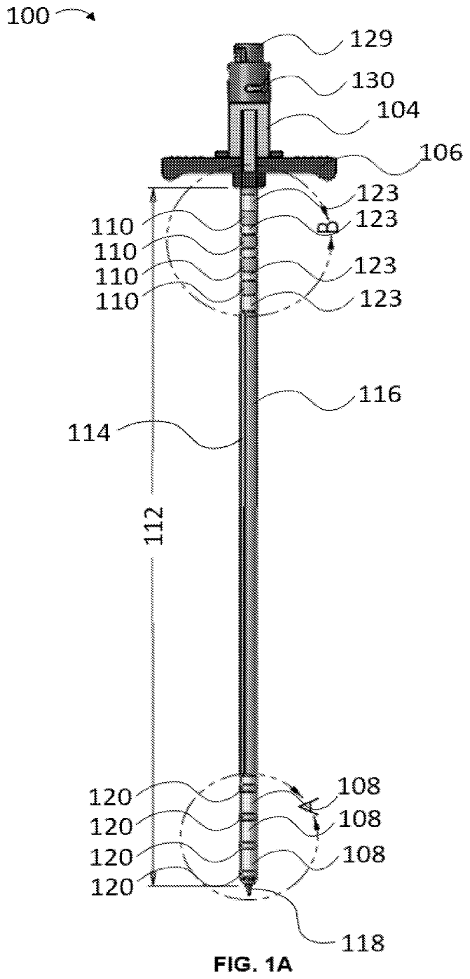

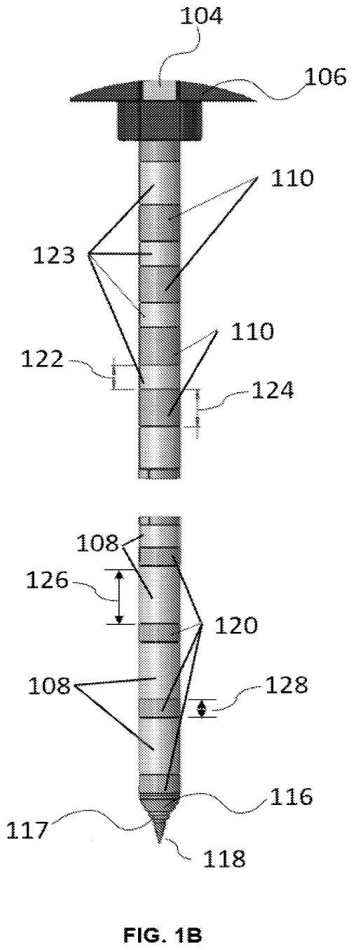

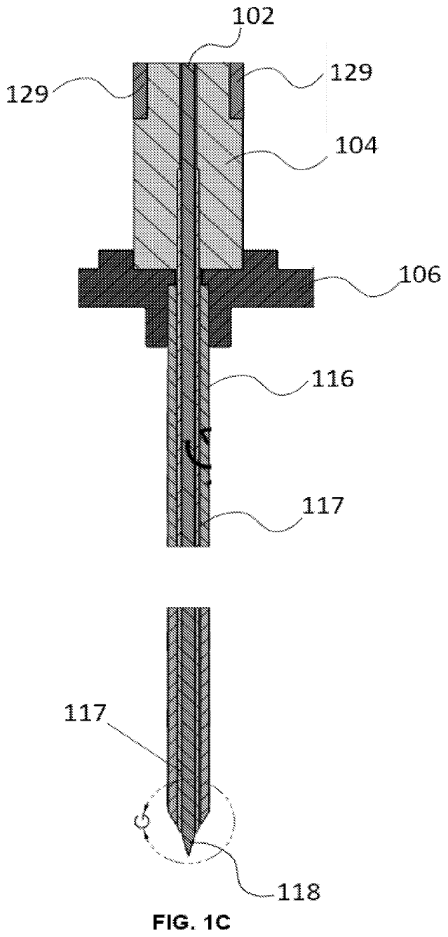

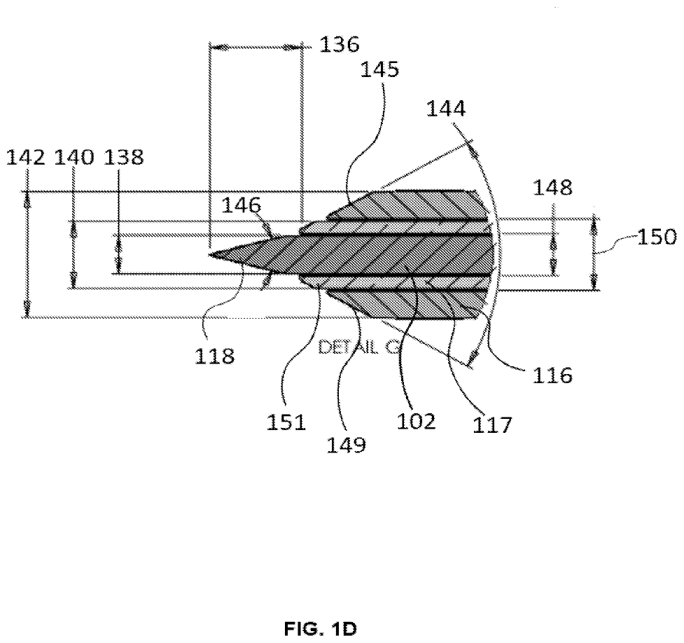

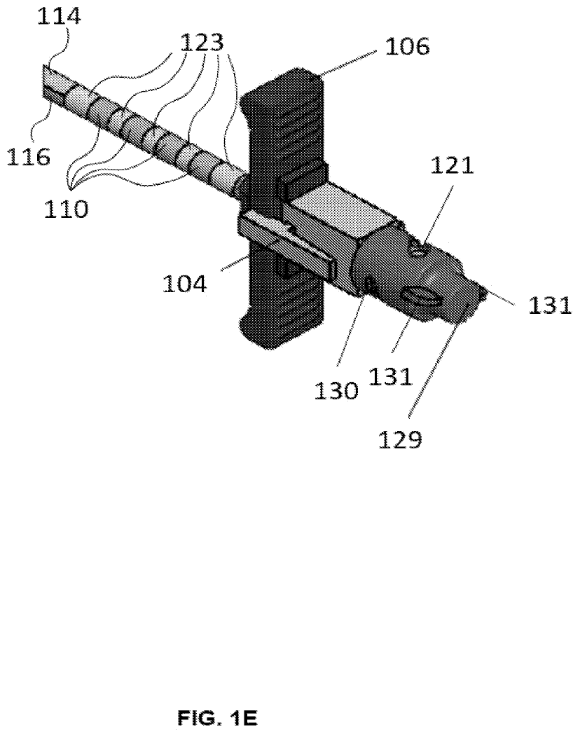

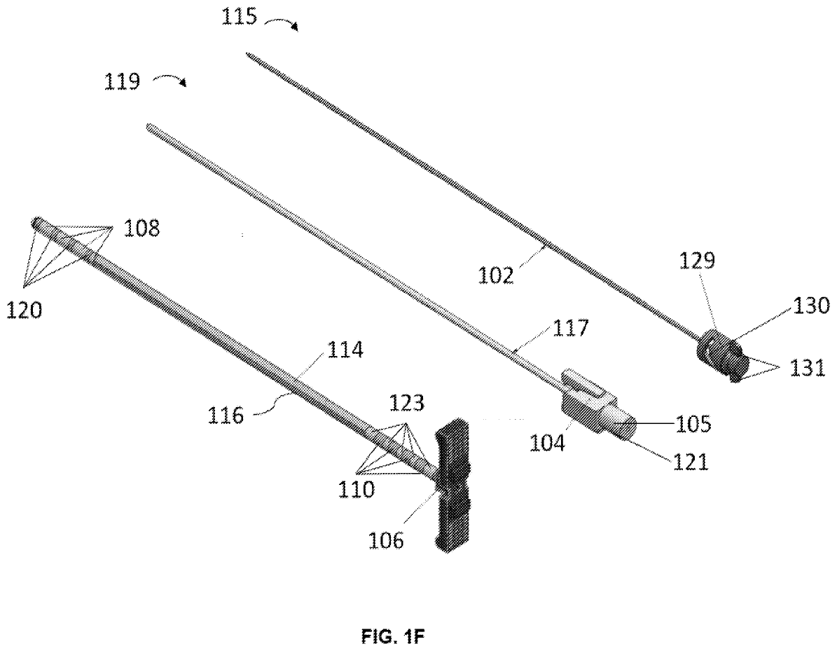

Provided herein are devices, systems, and methods for introducing and placing one or more electrode leads at one or more target sites for treatment of incontinence. In some embodiments, an electrode lead introducer used for placing the electrode at the target site comprises an introducer sheath, a dilator (also referred herein as an obturator), and a needle. FIGS. 1A-1F show an exemplary embodiment of the electrode lead introducer 100. In some cases, the electrode lead introducer may comprise an introducer sheath 114. an elongated body 116, an obturator 119, a needle 115, one or more electrodes (110, 120), one or more regions of electrode insulation (108, 123) or any combination thereof. In some cases, the elongated body 116 may be partially or wholly covered, coated, or surrounded by the introducer sheath 114.

In some embodiments, the introducer sheath comprises a sheath elongate shaft having a lumen and a sheath handle at a distal end of the sheath shaft. In some embodiments, the introducer sheath is configured to receive the electrode on an outer surface of the sheath elongate shaft. In some embodiments, a proximal end of the sheath elongate shaft is angled. In some embodiments, the angle of the proximal end of the sheath elongate shaft al-lows for advancing the device with little to no damage to surrounding tissue. In some embodiments, the introducer sheath has a diameter ranging from about 1 mm to about 5 mm.

In some embodiments, the obturator comprises an obturator elongate shaft having a lumen and an obturator handle at a distal end of the obturator elongate shaft. In some embodiments, the obturator elongate shaft is configured to fit inside the sheath lumen. In some embodiments, the obturator has a diameter ranging from about 1 mm to about 4 mm.

In some embodiments, the needle comprises a needle elongate shaft, a needle handle at a distal end of the needle elongate shaft, and a needle tip at the proximal end of the needle elongate shaft. In some embodiments, the needle elongate shaft is configured to fit inside the obturator lumen. In some embodiments, the obturator handle comprises a latch configured to attach to the sheath handle. In some embodiments, the needle tip is configured to protrude beyond an end of the obturator lumen. In some embodiments, the needle tip protrudes at least 1 mm beyond the end of the obturator lumen. In some embodiments, the needle tip is configured to protrude by a movement of the needle handle. In some embodiments, the needle tip is configured to be retractable into the obturator lumen. In some embodiments, the needle tip is angled from the needle elongate shaft. In some embodiments, the needle tip angle is configured to advance the needle tip through a soft tissue. In some embodiments, the needle has a diameter of about 0.4 mm to about 2 mm. In some embodiments, the needle has a diameter of between 12and 26 gauge.

In some cases, the elongated body 116 may be attached, fastened, and/or fused to a sheath handle 106 at one end of the elongated body 116, also referred herein as elongated shaft, as shown in FIGS. 1A, 1C, 1E, and 1F. In some instances, the introducer sheath 114 be integrated with the one or more electrodes and/or conductive regions (110, 120) and/or one or more regions of electrode insulation (108, 123). Alternatively or in combination, in some embodiments, the introducer sheath 114 may cover or surround, in whole or in part, non-conducting regions (108, 123) of one or more electrodes (110,120) integrated within the elongated body 116. In some instances, the introducer sheath 114 may comprise a non-conductive biocompatible material, including but not limited to high-density polyethylene (HDPE), fluorinated ethylene propylene (FEP), polycarbonate, plastics, or any combination thereof. In some instances, the introducer sheath 114 may be single use and/or disposable. Alternatively or in combination, the introducer sheath 114 may be autoclavable and/or may be cleaned by conventional sterilization methodologies used for other similar medical devices (i.e., trocars, endoscopes, etc.).

In some cases, the sheath handle 106 may be configured to allow a user, medical personal, and/or a surgeon to manipulate and/or navigate the electrode lead inserter as it is advanced into a patient or subject. In some cases, the sheath handle 106 may comprise an ergonomic geometry, where such ergonomic geometry may be configured to be operated with a single hand of a user, medical personal, and/or a surgeon, freeing up the other hand of the user, medical personal, and/or surgeon for other tasks.

In some cases, the mechanical stiffness of the material for the elongated body 116 and the introducer sheath 114 may be chosen to allow for easy insertion of the electrode lead introducer 100 into a patient. In some cases, the Young's modulus of the introducer sheath 114 and the elongated body 116 may allow for a user, medical personnel, and/or surgeon to maneuver the electrode lead inserter into a deep surgical plane to access the pudendal nerve. In some cases, the Young's modulus of the introducer sheath 114 and/or the elongated body 116 may prevent bowing or flexing of the combined elongated body 116 and introducer sheath when the user, medical personnel, and/or surgeon exerts a force on the distal end of the device when inserting into a patient. In some cases, the Young's modulus of the introducer sheath 114 and/or the elongated body 116 may lower the total mechanical work necessary to insert the electrode lead introducer into deep muscle and/or fat surgical planes adjacent to the pudendal nerve. In some instances, a higher stiffness of the sheath allows for easier delivery of the lead in regions with high tissue density or tissue resistance. In some instances, a higher stiffness of the sheath allows for more accurate placement of the lead to the target site in regions with high tissue density or tissue resistance around the target site. In some instances, the stiffness of the materials is characterized by Young's modulus. In some instances, the introducer sheath 114 has a Young's modulus of about 10 mega pascals (MPa) to about 10,000 MPa. In some instances, the elongated body 116 has a Young's modulus of about 10 MPa to about 10,000 MPa. In some instances, the sheath of the introducer to access the pudendal nerve has a higher stiffness than a sheath of an introducer typically used to access the sacral nerve. In some instances, the higher stiffness allows for easier access to the pudendal nerve and easier electrode lead placement on the anatomy of the pudendal nerve. In some instances, the combination of the sheath and needle has a stiffness similar to the combined sheath and dilator of an introducer typically used to access the sacral nerve. In some instances, the combination of the sheath and needle of the introducer has a higher stiffness than the combined sheath and dilator of an introducer typically used to access the sacral nerve.

In some cases, the elongated body 116 may comprise a length 112. In some cases, the length 112 of the device may enable proper manipulation of the device within patients comprising varying anatomical features, to properly place the one or more electrode leads, as described elsewhere herein. In some cases, the length of the refers to an insertable length. In some cases, the anatomical feature variations between subjects may comprise an enlargement or reduction in anatomical features surrounding or adjacent the pudendal, sacral nerves, or any combination or branches thereof.

In some cases, the length of the elongated body 116 may comprise a distance from about 10 centimeters (cm) to about 20 cm. In some cases, the length of the elongated body 116 may comprise a distances from about 12 cm to about 13 cm, about 12 cm to about 14 cm, about 12 cm to about 15 cm, about 12 cm to about 16 cm, about 12 cm to about 17 cm, about 12 cm to about 18 cm, about 12 cm to about 19 cm, about 12 cm to about 20 cm, about 13 cm to about 14 cm, about 13 cm to about 15 cm, about 13 cm to about 16 cm, about 13 cm to about 17 cm, about 13 cm to about 18 cm, about 13 cm to about 19 cm, about 13 cm to about 20 cm, about 14 cm to about 15 cm, about 14 cm to about 16 cm, about 14 cm to about 17 cm, about 14 cm to about 18 cm, about 14 cm to about 19 cm, about 14 cm to about 20 cm, about 15 cm to about 16 cm, about 15 cm to about 17 cm, about 15 cm to about 18 cm, about 15 cm to about 19 cm, about 15 cm to about 20 cm, about 16 cm to about 17 cm, about 16 cm to about 18 cm, about 16 cm to about 19 cm, about 16 cm to about 20 cm, about 17 cm to about 18 cm, about 17 cm to about 19 cm, about 17 cm to about 20 cm, about 18 cm to about 19 cm, about 18 cm to about 20 cm, or about 19 cm to about 20 cm. In some cases, the length of the elongated body 116 may comprise a distance from about 12 cm, about 13 cm, about 14 cm, about 15 cm, about 16 cm, about 17 cm, about 18 cm, about 19 cm, or about 20 cm. In some cases, the length of the elongated body 116 may comprise a distance from at least about 12 cm, about 13 cm, about 14 cm, about 15 cm, about 16 cm, about 17 cm, about 18 cm, or about 19 cm. In some cases, the length of the elongated body 116 may comprise a distance from at most about 13 cm, about 14 cm, about 15 cm, about 16 cm, about 17 cm, about 18 cm, about 19 cm, or about 20 cm.

FIG. 1D shows, in some embodiments, the elongated body 116, comprising an inner lumen diameter 150 that is configured to allow for the obturator 119 outer diameter 140 to pass through the inner lumen of the elongated body with a slip-fit mechanical interface.

In some cases, the elongated body inner lumen diameter 150 may comprise a distance of about 1.2 mm to about 3 mm. In some cases, the elongated body inner lumen diameter 150 may comprise a distance of about 1.2 millimeters (mm) to about 1.3 mm, about 1.2 mm to about 1.4 mm, about 1.2 mm to about 1.5 mm, about 1.2 mm to about 1.6 mm, about 1.2 mm to about 1.7 mm, about 1.2 mm to about 1.8 mm, about 1.2 mm to about 2 mm, about 1.2 mm to about 2.5 mm, about 1.2 mm to about 3 mm, about 1.3 mm to about 1.4 mm, about 1.3 mm to about 1.5 mm, about 1.3 mm to about 1.6 mm, about 1.3 mm to about 1.7 mm, about 1.3 mm to about 1.8 mm, about 1.3 mm to about 2 mm, about 1.3 mm to about 2.5 mm, about 1.3 mm to about 3 mm, about 1.4 mm to about 1.5 mm, about 1.4 mm to about 1.6 mm, about 1.4 mm to about 1.7 mm, about 1.4 mm to about 1.8 mm, about 1.4 mm to about 2 mm, about 1.4 mm to about 2.5 mm, about 1.4 mm to about 3 mm, about 1.5 mm to about 1.6 mm, about 1.5 mm to about 1.7 mm, about 1.5 mm to about 1.8 mm, about 1.5 mm to about 2 mm, about 1.5 mm to about 2.5 mm, about 1.5 mm to about 3 mm, about 1.6 mm to about 1.7 mm, about 1.6 mm to about 1.8 mm, about 1.6 mm to about 2 mm, about 1.6 mm to about 2.5 mm, about 1.6 mm to about 3 mm, about 1.7 mm to about 1.8 mm, about 1.7 mm to about 2 mm, about 1.7 mm to about 2.5 mm, about 1.7 mm to about 3 mm, about 1.8 mm to about 2 mm, about 1.8 mm to about 2.5 mm, about 1.8 mm to about 3 mm, about 2 mm to about 2.5 mm, about 2 mm to about 3 mm, or about 2.5 mm to about 3 mm. In some cases, the elongated body inner lumen diameter 150 may comprise a distance of about 1.2 mm, about 1.3 mm, about 1.4 mm, about 1.5 mm, about 1.6 mm, about 1.7 mm, about 1.8 mm, about 2 mm, about 2.5 mm, or about 3 mm. In some cases, the elongated body inner lumen diameter 150 may comprise a distance of at least about 1.2 mm, about 1.3 mm, about 1.4 mm, about 1.5 mm, about 1.6 mm, about 1.7 mm, about 1.8 mm, about 2 mm, or about 2.5 mm. In some cases, the inner lumen diameter 150 may comprise a distance of at most about 1.3 mm, about 1.4 mm, about 1.5 mm, about 1.6 mm, about 1.7 mm, about 1.8 mm, about 2 mm, about 2.5 mm, or about 3 mm. In some cases, the elongated body inner lumen diameter 150 may be a diameter that accept a guidewire such that the guidewire and the inner lumen diameter 150 comprise a slip-fit mechanical interface.

In some cases, the elongated body outer diameter 142 may comprise a diameter of about 1 mm to about 10 mm. In some cases, the elongated body outer diameter 142 may comprise a diameter of about 1 mm to about 1.5 mm, about 1 mm to about 2 mm, about 1 mm to about 2.5 mm, about 1 mm to about 3 mm, about 1 mm to about 3.5 mm, about 1 mm to about 4 mm, about 1 mm to about 4.5 mm, about 1 mm to about 5 mm, about 1 mm to about 6 mm, about 1 mm to about 8 mm, about 1 mm to about 10 mm, about 1.5 mm to about 2 mm, about 1.5 mm to about 2.5 mm, about 1.5 mm to about 3 mm, about 1.5 mm to about 3.5 mm, about 1.5 mm to about 4 mm, about 1.5 mm to about 4.5 mm, about 1.5 mm to about 5 mm, about 1.5 mm to about 6 mm, about 1.5 mm to about 8 mm, about 1.5 mm to about 10 mm, about 2 mm to about 2.5 mm, about 2 mm to about 3 mm, about 2 mm to about 3.5 mm, about 2 mm to about 4 mm, about 2 mm to about 4.5 mm, about 2 mm to about 5 mm, about 2 mm to about 6 mm, about 2 mm to about 8 mm, about 2 mm to about 10 mm, about 2.5 mm to about 3 mm, about 2.5 mm to about 3.5 mm, about 2.5 mm to about 4 mm, about 2.5 mm to about 4.5 mm, about 2.5 mm to about 5 mm, about 2.5 mm to about 6 mm, about 2.5 mm to about 8 mm, about 2.5 mm to about 10 mm, about 3 mm to about 3.5 mm, about 3 mm to about 4 mm, about 3 mm to about 4.5 mm, about 3 mm to about 5 mm, about 3 mm to about 6 mm, about 3 mm to about 8 mm, about 3 mm to about 10 mm, about 3.5 mm to about 4 mm, about 3.5 mm to about 4.5 mm, about 3.5 mm to about 5 mm, about 3.5 mm to about 6 mm, about 3.5 mm to about 8 mm, about 3.5 mm to about 10 mm, about 4 mm to about 4.5 mm, about 4 mm to about 5 mm, about 4 mm to about 6 mm, about 4 mm to about 8 mm, about 4 mm to about 10 mm, about 4.5 mm to about 5 mm, about 4.5 mm to about 6 mm, about 4.5 mm to about 8 mm, about 4.5 mm to about 10 mm, about 5 mm to about 6 mm, about 5 mm to about 8 mm, about 5 mm to about 10 mm, about 6 mm to about 8 mm, about 6 mm to about 10 mm, or about 8 mm to about 10 mm. In some cases, the elongated body outer diameter 142 may comprise a diameter of about 1 mm, about 1.5 mm, about 2 mm, about 2.5 mm, about 3 mm, about 3.5 mm, about 4 mm, about 4.5 mm, about 5 mm, about 6 mm, about 8 mm, or about 10 mm. In some cases, the elongated body outer diameter 142 may comprise a diameter of at least about 1 mm, about 1.5 mm, about 2 mm, about 2.5 mm, about 3 mm, about 3.5 mm, about 4 mm, about 4.5 mm, about 5 mm, about 6 mm, or about 8 mm. In some cases, the elongated body outer diameter 142 may comprise a diameter of at most about 1.5 mm, about 2 mm, about 2.5 mm, about 3 mm, about 3.5 mm, about 4 mm, about 4.5 mm, about 5 mm, about 6 mm, about 8 mm, or about 10 mm.

In some embodiments, the elongated body 116 may comprise an angled facet 145 on one end of the elongated body 116, as shown in FIG. 1D. In some instances, the angled facet 145 of the elongated body 116 may be configured to allow for the electrode lead inserter 100 to penetrate into a subject receiving an implanted electrode in a similar manner as a pointed needle. In some embodiments, the angle facet 145 of the proximal end of the sheath elongate shaft may allow for advancing the device with little to no damage to surrounding tissue. In some cases, the angled facet 145 may be angled at an angle 144 with respect to a mirrored angled facet 149 of the elongated body 116 separated by 180 degrees.

In some cases, the angle 144 may comprise a value of about 30 degrees to about 90 degrees. In some cases, the angle 144 may comprise a value of about 30 degrees to about 45 degrees, about 30 degrees to about 60 degrees, about 30 degrees to about 75 degrees, about 30 degrees to about 90 degrees, about 45 degrees to about 60 degrees, about 45 degrees to about 75 degrees, about 45 degrees to about 90 degrees, about 60 degrees to about 75 degrees, about 60 degrees to about 90 degrees, about 75 degrees to about 90 degrees, or about 80 degrees to about 90 degrees. In some cases, the angle 144 may comprise a value of about 30 degrees, about 35 degrees, about 40 degrees, about 45 degrees, about 50 degrees, about 55 degrees, about 60 degrees, about 65 degrees, about 70 degrees, about 75 degrees, about 80 degrees, or about 90 degrees. In some cases, the angle 144 may comprise a value of at least about 30 degrees, about 35 degrees, about 40 degrees, about 45 degrees, about 50 degrees, about 55 degrees, about 60 degrees, about 65 degrees, about 70 degrees, about 75 degrees, or about 80 degrees. In some cases, the angle 144 may comprise a value of at most about 35 degrees, about 40 degrees, about 45 degrees, about 50 degrees, about 55 degrees, about 60 degrees, about 65 degrees, about 70 degrees, about 75 degrees, about 80 degrees, or about 90 degrees.

In some embodiments, the obturator 119 may comprises an obturator elongated body 117 having a lumen and an obturator handle 104 at an end of the obturator elongated body, as shown in FIG. 1F. In some instances, the obturator elongated body 117 may be comprised of a plastic (e.g., ABS), stainless steel, or any combination thereof. In some cases, the obturator may be comprised of a stainless-steel obturator elongated body 117 yet comprise a plastic handle 104. In some cases, the obturator elongated body 117 may be configured to fit inside the elongated body 116 lumen.

In some instances, the obturator elongated body 117 may comprise an outer diameter 140. In some cases, the outer diameter 140 of the obturator elongated body 117 may comprise a diameter of about 0.5 mm to about 5 mm. In some cases, the outer diameter 140 of the obturator elongated body 117 may comprise a diameter of about 0.5 mm to about 0.6 mm, about 0.5 mm to about 0.7 mm, about 0.5 mm to about 0.8 mm, about 0.5 mm to about 0.9 mm, about 0.5 mm to about 1 mm, about 0.5 mm to about 1.5 mm, about 0.5 mm to about 2 mm, about 0.5 mm to about 2.5 mm, about 0.5 mm to about 3 mm, about 0.5 mm to about 4 mm, about 0.5 mm to about 5 mm, about 0.6 mm to about 0.7 mm, about 0.6 mm to about 0.8 mm, about 0.6 mm to about 0.9 mm, about 0.6 mm to about 1 mm, about 0.6 mm to about 1.5 mm, about 0.6 mm to about 2 mm, about 0.6 mm to about 2.5 mm, about 0.6 mm to about 3 mm, about 0.6 mm to about 4 mm, about 0.6 mm to about 5 mm, about 0.7 mm to about 0.8 mm, about 0.7 mm to about 0.9 mm, about 0.7 mm to about 1 mm, about 0.7 mm to about 1.5 mm, about 0.7 mm to about 2 mm, about 0.7 mm to about 2.5 mm, about 0.7 mm to about 3 mm, about 0.7 mm to about 4 mm, about 0.7 mm to about 5 mm, about 0.8 mm to about 0.9 mm, about 0.8 mm to about 1 mm, about 0.8 mm to about 1.5 mm, about 0.8 mm to about 2 mm, about 0.8 mm to about 2.5 mm, about 0.8 mm to about 3 mm, about 0.8 mm to about 4 mm, about 0.8 mm to about 5 mm, about 0.9 mm to about 1 mm, about 0.9 mm to about 1.5 mm, about 0.9 mm to about 2 mm, about 0.9 mm to about 2.5 mm, about 0.9 mm to about 3 mm, about 0.9 mm to about 4 mm, about 0.9 mm to about 5 mm, about 1 mm to about 1.5 mm, about 1 mm to about 2 mm, about 1 mm to about 2.5 mm, about 1 mm to about 3 mm, about 1 mm to about 4 mm, about 1 mm to about 5 mm, about 1.5 mm to about 2 mm, about 1.5 mm to about 2.5 mm, about 1.5 mm to about 3 mm, about 1.5 mm to about 4 mm, about 1.5 mm to about 5 mm, about 2 mm to about 2.5 mm, about 2 mm to about 3 mm, about 2 mm to about 4 mm, about 2 mm to about 5 mm, about 2.5 mm to about 3 mm, about 2.5 mm to about 4 mm, about 2.5 mm to about 5 mm, about 3 mm to about 4 mm, about 3 mm to about 5 mm, or about 4 mm to about 5 mm. In some cases, the outer diameter 140 of the obturator elongated body 117 may comprise a diameter of about 0.5 mm, about 0.6 mm, about 0.7 mm, about 0.8 mm, about 0.9 mm, about 1 mm, about 1.5 mm, about 2 mm, about 2.5 mm, about 3 mm, about 4 mm, or about 5 mm. In some cases, the outer diameter 140 of the obturator elongated body 117 may comprise a diameter of at least about 0.5 mm, about 0.6 mm, about 0.7 mm, about 0.8 mm, about 0.9 mm, about 1 mm, about 1.5 mm, about 2 mm, about 2.5 mm, about 3 mm, or about 4 mm. In some cases, the outer diameter 140 of the obturator elongated body 117 may comprise a diameter of at most about 0.6 mm, about 0.7 mm, about 0.8 mm, about 0.9 mm, about 1 mm, about 1.5 mm, about 2 mm, about 2.5 mm, about 3 mm, about 4 mm, or about 5 mm.

In some cases, the obturator 119 may comprise an inner lumen, as seen in FIG. 1D. In some instances, the inner lumen may comprise an inner diameter 148. In some instances, the inner lumen of the obturator 119 may comprise a diameter such that the needle body 102 and obturator inner lumen mechanically coupled with a slip fit interface.

In some cases, the inner diameter 148 of the inner lumen of the obturator 119 may comprise a diameter of about 0.2 mm to about 1.4 mm. In some cases, the inner diameter 148 of the inner lumen of the obturator 119 may comprise a diameter of about 0.2 mm to about 0.3 mm, about 0.2 mm to about 0.4 mm, about 0.2 mm to about 0.5 mm, about 0.2 mm to about 0.6 mm, about 0.2 mm to about 0.7 mm, about 0.2 mm to about 0.8 mm, about 0.2 mm to about 0.9 mm, about 0.2 mm to about 1 mm, about 0.2 mm to about 1.2 mm, about 0.2 mm to about 1.4 mm, about 0.3 mm to about 0.4 mm, about 0.3 mm to about 0.5 mm, about 0.3 mm to about 0.6 mm, about 0.3 mm to about 0.7 mm, about 0.3 mm to about 0.8 mm, about 0.3 mm to about 0.9 mm, about 0.3 mm to about 1 mm, about 0.3 mm to about 1.2 mm, about 0.3 mm to about 1.4 mm, about 0.4 mm to about 0.5 mm, about 0.4 mm to about 0.6 mm, about 0.4 mm to about 0.7 mm, about 0.4 mm to about 0.8 mm, about 0.4 mm to about 0.9 mm, about 0.4 mm to about 1 mm, about 0.4 mm to about 1.2 mm, about 0.4 mm to about 1.4 mm, about 0.5 mm to about 0.6 mm, about 0.5 mm to about 0.7 mm, about 0.5 mm to about 0.8 mm, about 0.5 mm to about 0.9 mm, about 0.5 mm to about 1 mm, about 0.5 mm to about 1.2 mm, about 0.5 mm to about 1.4 mm, about 0.6 mm to about 0.7 mm, about 0.6 mm to about 0.8 mm, about 0.6 mm to about 0.9 mm, about 0.6 mm to about 1 mm, about 0.6 mm to about 1.2 mm, about 0.6 mm to about 1.4 mm, about 0.7 mm to about 0.8 mm, about 0.7 mm to about 0.9 mm, about 0.7 mm to about 1 mm, about 0.7 mm to about 1.2 mm, about 0.7 mm to about 1.4 mm, about 0.8 mm to about 0.9 mm, about 0.8 mm to about 1 mm, about 0.8 mm to about 1.2 mm, about 0.8 mm to about 1.4 mm, about 0.9 mm to about 1 mm, about 0.9 mm to about 1.2 mm, about 0.9 mm to about 1.4 mm, about 1 mm to about 1.2 mm, about 1 mm to about 1.4 mm, or about 1.2 mm to about 1.4 mm. In some cases, the inner diameter 148 of the inner lumen of the obturator 119 may comprise a diameter of about 0.2 mm, about 0.3 mm, about 0.4 mm, about 0.5 mm, about 0.6 mm, about 0.7 mm, about 0.8 mm, about 0.9 mm, about 1 mm, about 1.2 mm, or about 1.4 mm. In some cases, the inner diameter 148 of the inner lumen of the obturator 119 may comprise a diameter of at least about 0.2 mm, about 0.3 mm, about 0.4 mm, about 0.5 mm, about 0.6 mm, about 0.7 mm, about 0.8 mm, about 0.9 mm, about 1 mm, or about 1.2 mm. In some cases, the inner diameter 148 of the inner lumen of the obturator 119 may comprise a diameter of at most about 0.3 mm, about 0.4 mm, about 0.5 mm, about 0.6 mm, about 0.7 mm, about 0.8 mm, about 0.9 mm, about 1 mm, about 1.2 mm, or about 1.4 mm.

In some cases, the obturator 119 may be configured to assist the insertion of the elongated body 116 into the patient/subject receiving the electrode lead implant by providing structural rigidity. In some cases, the obturator elongated body 117 may comprise a region 151 that protrudes a distance out from the elongated body 116, as seen in FIG. 1D. In some cases, the region 151 that protrudes a distance out from the elongated body 116 may comprise a blunt protrusion. Alternatively or in combination, the region 151 that protrudes a distance out from the elongated body 116 may comprise a protrusion with angle parallel to that of the angle facet 145 of the elongated body 116, described elsewhere herein.

In some instances, the distance of the protrusion 151 that may extend beyond the end of the elongated body 116 may extend by a distance of about 0.2 mm to about 3 mm. In some instances, the distance of the protrusion 151 that may extend beyond the end of the elongated body 116 may extend by a distance of about 0.2 mm to about 0.4 mm, about 0.2 mm to about 0.6 mm, about 0.2 mm to about 0.8 mm, about 0.2 mm to about 1 mm, about 0.2 mm to about 1.2 mm, about 0.2 mm to about 1.4 mm, about 0.2 mm to about 1.6 mm, about 0.2 mm to about 1.8 mm, about 0.2 mm to about 2 mm, about 0.2 mm to about 2.5 mm, about 0.2 mm to about 3 mm, about 0.4 mm to about 0.6 mm, about 0.4 mm to about 0.8 mm, about 0.4 mm to about 1 mm, about 0.4 mm to about 1.2 mm, about 0.4 mm to about 1.4 mm, about 0.4 mm to about 1.6 mm, about 0.4 mm to about 1.8 mm, about 0.4 mm to about 2 mm, about 0.4 mm to about 2.5 mm, about 0.4 mm to about 3 mm, about 0.6 mm to about 0.8 mm, about 0.6 mm to about 1 mm, about 0.6 mm to about 1.2 mm, about 0.6 mm to about 1.4 mm, about 0.6 mm to about 1.6 mm, about 0.6 mm to about 1.8 mm, about 0.6 mm to about 2 mm, about 0.6 mm to about 2.5 mm, about 0.6 mm to about 3 mm, about 0.8 mm to about 1 mm, about 0.8 mm to about 1.2 mm, about 0.8 mm to about 1.4 mm, about 0.8 mm to about 1.6 mm, about 0.8 mm to about 1.8 mm, about 0.8 mm to about 2 mm, about 0.8 mm to about 2.5 mm, about 0.8 mm to about 3 mm, about 1 mm to about 1.2 mm, about 1 mm to about 1.4 mm, about 1 mm to about 1.6 mm, about 1 mm to about 1.8 mm, about 1 mm to about 2 mm, about 1 mm to about 2.5 mm, about 1 mm to about 3 mm, about 1.2 mm to about 1.4 mm, about 1.2 mm to about 1.6 mm, about 1.2 mm to about 1.8 mm, about 1.2 mm to about 2 mm, about 1.2 mm to about 2.5 mm, about 1.2 mm to about 3 mm, about 1.4 mm to about 1.6 mm, about 1.4 mm to about 1.8 mm, about 1.4 mm to about 2 mm, about 1.4 mm to about 2.5 mm, about 1.4 mm to about 3 mm, about 1.6 mm to about 1.8 mm, about 1.6 mm to about 2 mm, about 1.6 mm to about 2.5 mm, about 1.6 mm to about 3 mm, about 1.8 mm to about 2 mm, about 1.8 mm to about 2.5 mm, about 1.8 mm to about 3 mm, about 2 mm to about 2.5 mm, about 2 mm to about 3 mm, or about 2.5 mm to about 3 mm. In some instances, the distance of the protrusion 151 that may extend beyond the end of the elongated body 116 may extend by a distance of about 0.2 mm, about 0.4 mm, about 0.6 mm, about 0.8 mm, about 1 mm, about 1.2 mm, about 1.4 mm, about 1.6 mm, about 1.8 mm, about 2 mm, about 2.5 mm, or about 3 mm. In some instances, the distance of the protrusion 151 that may extend beyond the end of the elongated body 116 may extend by a distance of at least about 0.2 mm, about 0.4 mm, about 0.6 mm, about 0.8 mm, about 1 mm, about 1.2 mm, about 1.4 mm, about 1.6 mm, about 1.8 mm, about 2 mm, or about 2.5 mm. In some instances, the distance of the protrusion 151 that may extend beyond the end of the elongated body 116 may extend by a distance of at most about 0.4 mm, about 0.6 mm, about 0.8 mm, about 1 mm, about 1.2 mm, about 1.4 mm, about 1.6 mm, about 1.8 mm, about 2 mm, about 2.5 mm, or about 3 mm.

In some cases, the obturator handle 104 may mechanically couple to the sheath handle 106, as shown in FIG. 1A and FIG. 1E. In some cases, the mechanical coupling between the obturator handle 104 and the sheath handle 106 may comprise a hook and latch, quick release, or any combination thereof. In some cases, the obturator handle 104 may comprise a coupling receptacle 105, configured to receive and fasten to the coupling mechanism of the needle handle 129, as seen in FIG. 1E. In some cases, the coupling receptacle 105 may comprise a coupling feature 121 configured to interface with the needle handle 129 when the needle body 102 is inserted into the inner lumen of obturator elongated body 117. In some instances, the coupling feature 121 may be configured to slide within a track on the needle handle 129. In some cases, the coupling feature 121 may interface with the track of the needle handle 129 at a location of the track with an interference fit, thereby providing an interference fit based mechanical fastening between the needle 115 and the obturator 119.

In some embodiments, the needle 115 may comprises a needle elongated body 102, a needle handle 129 at one end of the needle elongate shaft, and a needle tip 118 at the other end of the needle elongate shaft. In some cases, the needle elongated body 102 may be configured to fit into the obturator inner lumen, designated by an inner diameter 148 of the obturator inner lumen. In some cases, the needle elongated body may comprise an outer diameter 138, as seen in FIG. 1D. In some instances, the fit may comprise a slip fit between the inner diameter 148 of the obturator inner lumen and the outer diameter 138 of the needle elongated body. In some cases, the needle 115 may be comprised of stainless steel. In some instances, the needle 115 may be hollow or maybe be partially hollow and/or partially solid. Alternatively or in combination, the needle 115 may be comprised of a rigid non-deformable plastic and/or polymer.

In some instances, the needle elongated body outer diameter 138 may comprise a diameter of about 0.1 mm to about 3 mm. In some instances, the needle elongated body outer diameter 138 may comprise a diameter of about 0.1 mm to about 0.2 mm, about 0.1 mm to about 0.3 mm, about 0.1 mm to about 0.4 mm, about 0.1 mm to about 0.5 mm, about 0.1 mm to about 0.6 mm, about 0.1 mm to about 0.8 mm, about 0.1 mm to about 1 mm, about 0.1 mm to about 1.5 mm, about 0.1 mm to about 2 mm, about 0.1 mm to about 2.5 mm, about 0.1 mm to about 3 mm, about 0.2 mm to about 0.3 mm, about 0.2 mm to about 0.4 mm, about 0.2 mm to about 0.5 mm, about 0.2 mm to about 0.6 mm, about 0.2 mm to about 0.8 mm, about 0.2 mm to about 1 mm, about 0.2 mm to about 1.5 mm, about 0.2 mm to about 2 mm, about 0.2 mm to about 2.5 mm, about 0.2 mm to about 3 mm, about 0.3 mm to about 0.4 mm, about 0.3 mm to about 0.5 mm, about 0.3 mm to about 0.6 mm, about 0.3 mm to about 0.8 mm, about 0.3 mm to about 1 mm, about 0.3 mm to about 1.5 mm, about 0.3 mm to about 2 mm, about 0.3 mm to about 2.5 mm, about 0.3 mm to about 3 mm, about 0.4 mm to about 0.5 mm, about 0.4 mm to about 0.6 mm, about 0.4 mm to about 0.8 mm, about 0.4 mm to about 1 mm, about 0.4 mm to about 1.5 mm, about 0.4 mm to about 2 mm, about 0.4 mm to about 2.5 mm, about 0.4 mm to about 3 mm, about 0.5 mm to about 0.6 mm, about 0.5 mm to about 0.8 mm, about 0.5 mm to about 1 mm, about 0.5 mm to about 1.5 mm, about 0.5 mm to about 2 mm, about 0.5 mm to about 2.5 mm, about 0.5 mm to about 3 mm, about 0.6 mm to about 0.8 mm, about 0.6 mm to about 1 mm, about 0.6 mm to about 1.5 mm, about 0.6 mm to about 2 mm, about 0.6 mm to about 2.5 mm, about 0.6 mm to about 3 mm, about 0.8 mm to about 1 mm, about 0.8 mm to about 1.5 mm, about 0.8 mm to about 2 mm, about 0.8 mm to about 2.5 mm, about 0.8 mm to about 3 mm, about 1 mm to about 1.5 mm, about 1 mm to about 2 mm, about 1 mm to about 2.5 mm, about 1 mm to about 3 mm, about 1.5 mm to about 2 mm, about 1.5 mm to about 2.5 mm, about 1.5 mm to about 3 mm, about 2 mm to about 2.5 mm, about 2 mm to about 3 mm, or about 2.5 mm to about 3 mm. In some instances, the needle elongated body outer diameter 138 may comprise a diameter of about 0.1 mm, about 0.2 mm, about 0.3 mm, about 0.4 mm, about 0.5 mm, about 0.6 mm, about 0.8 mm, about 1 mm, about 1.5 mm, about 2 mm, about 2.5 mm, or about 3 mm. In some instances, the needle elongated body outer diameter 138 may comprise a diameter of at least about 0.1 mm, about 0.2 mm, about 0.3 mm, about 0.4 mm, about 0.5 mm, about 0.6 mm, about 0.8 mm, about 1 mm, about 1.5 mm, about 2 mm, or about 2.5 mm. In some instances, the needle elongated body outer diameter 138 may comprise a diameter of at most about 0.2 mm, about 0.3 mm, about 0.4 mm, about 0.5 mm, about 0.6 mm, about 0.8 mm, about 1 mm, about 1.5 mm, about 2 mm, about 2.5 mm, or about 3 mm.

In some instances, the needle elongated body outer diameter 138 may comprise a diameter of about 12 American Wire Gauge (AWG) to about 26 AWG. In some instances, the needle elongated body outer diameter 138 may comprise a diameter of about 12 AWG to about 14 AWG, about 12 AWG to about 16 AWG, about 12 AWG to about 18 AWG, about 12 AWG to about 20 AWG, about 12 AWG to about 22 AWG, about 12 AWG to about 24 AWG, about 12 AWG to about 26 AWG, about 14 AWG to about 16 AWG, about 14 AWG to about 18 AWG, about 14 AWG to about 20 AWG, about 14 AWG to about 22 AWG, about 14 AWG to about 24 AWG, about 14 AWG to about 26 AWG, about 16 AWG to about 18 AWG, about 16 AWG to about 20 AWG, about 16 AWG to about 22 AWG, about 16 AWG to about 24 AWG, about 16 AWG to about 26 AWG, about 18 AWG to about 20 AWG, about 18 AWG to about 22 AWG, about 18 AWG to about 24 AWG, about 18 AWG to about 26 AWG, about 20 AWG to about 22 AWG, about 20 AWG to about 24 AWG, about 20 AWG to about 26 AWG, about 22 AWG to about 24 AWG, about 22 AWG to about 26 AWG, or about 24 AWG to about 26 AWG. In some instances, the needle elongated body outer diameter 138 may comprise a diameter of about 12 AWG, about 14 AWG, about 16 AWG, about 18 AWG, about 20 AWG, about 22 AWG, about 24 AWG, or about 26 AWG. In some instances, the needle elongated body outer diameter 138 may comprise a diameter of at least about 12 AWG, about 14 AWG, about 16 AWG, about 18 AWG, about 20 AWG, about 22 AWG, or about 24 AWG. In some instances, the needle elongated body outer diameter 138 may comprise a diameter of at most about 14 AWG, about 16 AWG, about 18 AWG, about 20 AWG, about 22 AWG, about 24 AWG, or about 26 AWG.

In some cases, the needle handle 129 may comprise a coupling feature 130 configured to couple to the coupling feature 121 of the obturator, described elsewhere herein. In some cases, the needle handle coupling feature 130 may comprise a path or a slot feature, whereby the obturator coupling feature 121, upon inserting the needle 115 into the inner lumen of the obturator, may travel within and apply a tension and/or holding force when rotated around the central axis of the needle 115 and obturator 119. In some instances, the needle handle may be rotated by rotating one or more flanges 131 of the needle handle 129. In some cases, the rotation may be accomplished by the user, medical personal, surgeon or any combination thereof personnel. Alternatively or in combination, the rotation may be accomplished by a motor.

In some embodiments, the needle tip 118 may configured to protrude beyond an end of the obturator lumen. In some embodiments, the needle tip 118 may protrude 136 at least 1 mm beyond the end of the obturator lumen. In some embodiments, the needle tip may be configured to protrude by a movement of the needle handle 129. In some embodiments, the needle tip 118 may be configured to be retractable into the obturator lumen.

In some embodiments, the needle tip 118 may have an angle 146 ranging from about 15 degrees to about 45 degrees from the needle elongated body about 1 degree to about 50 degrees. In some embodiments, the needle tip 118 may have an angle 146 ranging from about 15 degrees to about 45 degrees from the needle elongated body about 15 degrees to about 25 degrees, about 15 degrees to about 35 degrees, about 15 degrees to about 40 degrees, about 15 degrees to about 45 degrees, about 15 degrees to about 50 degrees, about 15 degrees to about 1 degree, about 25 degrees to about 35 degrees, about 25 degrees to about 40 degrees, about 25 degrees to about 45 degrees, about 25 degrees to about 50 degrees, about 25 degrees to about 1 degree, about 35 degrees to about 40 degrees, about 35 degrees to about 45 degrees, about 35 degrees to about 50 degrees, about 35 degrees to about 1 degree, about 40 degrees to about 45 degrees, about 40 degrees to about 50 degrees, about 40 degrees to about 1 degree, about 45 degrees to about 50 degrees, about 45 degrees to about 1 degree, or about 50 degrees to about 1 degree. In some embodiments, the needle tip 118 may have an angle 146 ranging from about 15 degrees to about 45 degrees from the needle elongated body about 15 degrees, about 25 degrees, about 35 degrees, about 40 degrees, about 45 degrees, about 50 degrees, or about 1 degree. In some embodiments, the needle tip 118 may have an angle 146 ranging from about 15 degrees to about 45 degrees from the needle elongated body at least about 15 degrees, about 25 degrees, about 35 degrees, about 40 degrees, about 45 degrees, or about 50 degrees. In some embodiments, the needle tip 118 may have an angle 146 ranging from about 15 degrees to about 45 degrees from the needle elongated body at most about 25 degrees, about 35 degrees, about 40 degrees, about 45 degrees, about 50 degrees, or about 1 degree. In some embodiments, the needle tip 118 angle 146 may be configured to advance the needle tip through a soft tissue.

In some embodiments, the electrode lead introducer 100 may comprise one or more electrodes (120, 110), as seen in FIGS. 1A, 1B, 1E, and 1F, configured to provide electrical stimulation and/or to measure electrical signals adjacent to a patient's pudendal, sacral, or any combination thereof nerves or any branches thereof. In some instances, the electrodes may be configured to assist the user, medical personal, and/or surgeon, in navigating to target portions of the patient's pudendal, sacral, or any combination thereof nerves or any branches thereof, that produce. In some cases, the electrodes may be adjacent to one or more insulators (108, 123). The one or more insulators (108, 123) may be configured to not conduct and/or sense electrical current.

In some cases, the electrode lead introducer 100 may comprise two sets of one or more electrodes (120, 110) and two sets of one or more insulators (108, 123) whereby each set of electrodes and/or insulators are located at opposite ends of the electrode lead introducer, as seen in FIGS. 1A-1B. Designating for purposes of example only, the needle tip 118 as the proximal end of the electrode lead introducer 100, the first set of one or more electrodes 120 and/or the first set of one or more insulators 108 may be located at a proximal end of the electrode lead introducer, as shown in FIG. 1B. Designating for purposes of example only, the sheath handle 106 as the distal end of the electrode lead introducer 100, the second set of one or more electrodes 110 and/or the second set of one or more insulators 123 may be located at a distal end of the electrode lead introducer. In some instances, the one or more electrodes located at the proximal end of the electrode lead introducer 120 may be in electrical communication with the one or more electrodes located at the distal end of the electrode lead introducer 110. In some cases, the proximal end one or more electrodes 120 may be configured to detect and/or provide electrical signals to a patient's pudendal, sacral, or any combination thereof nerves or any branches thereof. In some cases, the one or more electrodes (120, 110) at the distal and/or proximal region of the electrode lead introducer may comprise at least one, at least two, at least three, at least four, at least five, or at least six electrodes. In some cases, the one or more electrodes (120, 110) at the distal and/or proximal region of the electrode lead introducer may comprise at most one, at most two, at most three, at most four, at most five, or at most six electrodes. In some cases, the distal one or more electrodes may be configured to couple to terminations of a hook type probe, where the hook type probe may provide an electrical stimulation signal and/or detect an electrical signal via the one or more electrodes at the distal end 120. In some cases, the hook type probe may be in electrical communication with the one or more distal and/or proximal electrodes.

In some instances, the one or more electrodes located at the distal 110 and proximal 120 end and/or the one or more insulators located at the distal 120 and proximal 108 end of the electrode lead introducer may comprise electrodes and insulators of varying length, as can be seen in FIG. 1B. In some instances, the one or more proximal electrodes 120, may comprise a length 128. In some cases, the length 128 of the proximal one or more electrodes 120 may comprise about 0.8 mm to about 2 mm. In some cases, the length 128 of the proximal one or more electrodes 120 may comprise about 0.8 mm to about 0.9 mm, about 0.8 mm to about 1 mm, about 0.8 mm to about 1.1 mm, about 0.8 mm to about 1.2 mm, about 0.8 mm to about 1.3 mm, about 0.8 mm to about 1.4 mm, about 0.8 mm to about 1.5 mm, about 0.8 mm to about 2 mm, about 0.9 mm to about 1 mm, about 0.9 mm to about 1.1 mm, about 0.9 mm to about 1.2 mm, about 0.9 mm to about 1.3 mm, about 0.9 mm to about 1.4 mm, about 0.9 mm to about 1.5 mm, about 0.9 mm to about 2 mm, about 1 mm to about 1.1 mm, about 1 mm to about 1.2 mm, about 1 mm to about 1.3 mm, about 1 mm to about 1.4 mm, about 1 mm to about 1.5 mm, about 1 mm to about 2 mm, about 1.1 mm to about 1.2 mm, about 1.1 mm to about 1.3 mm, about 1.1 mm to about 1.4 mm, about 1.1 mm to about 1.5 mm, about 1. 1 mm to about 2 mm, about 1.2 mm to about 1.3 mm, about 1.2 mm to about 1.4 mm, about 1.2 mm to about 1.5 mm, about 1.2 mm to about 2 mm, about 1.3 mm to about 1.4 mm, about 1.3 mm to about 1.5 mm, about 1.3 mm to about 2 mm, about 1.4 mm to about 1.5 mm, about 1.4 mm to about 2 mm, or about 1.5 mm to about 2 mm. In some cases, the length 128 of the proximal one or more electrodes 120 may comprise about 0.8 mm, about 0.9 mm, about 1 mm, about 1.1 mm, about 1.2 mm, about 1.3 mm, about 1.4 mm, about 1.5 mm, or about 2 mm. In some cases, the length 128 of the proximal one or more electrodes 120 may comprise at least about 0.8 mm, about 0.9 mm, about 1 mm, about 1.1 mm, about 1.2 mm, about 1.3 mm, about 1.4 mm, or about 1.5 mm. In some cases, the length 128 of the proximal one or more electrodes 120 may comprise at most about 0.9 mm, about 1 mm, about 1. 1 mm, about 1.2 mm, about 1.3 mm, about 1.4 mm, about 1.5 mm, or about 2 mm.

In some cases, the length 126 of the proximal one or more insulators 108 may comprise about 5 mm to about 7 mm. In some cases, the length 126 of the proximal one or more insulators 108 may comprise about 5 mm to about 5.1 mm, about 5 mm to about 5.2 mm, about 5 mm to about 5.3 mm, about 5 mm to about 5.4 mm, about 5 mm to about 5.5 mm, about 5 mm to about 5.8 mm, about 5 mm to about 6 mm, about 5 mm to about 6.5 mm, about 5 mm to about 7 mm, about 5.1 mm to about 5.2 mm, about 5.1 mm to about 5.3 mm, about 5.1 mm to about 5.4 mm, about 5. 1 mm to about 5.5 mm, about 5.1 mm to about 5.8 mm, about 5.1 mm to about 6 mm, about 5. 1 mm to about 6.5 mm, about 5.1 mm to about 7 mm, about 5.2 mm to about 5.3 mm, about 5.2 mm to about 5.4 mm, about 5.2 mm to about 5.5 mm, about 5.2 mm to about 5.8 mm, about 5.2 mm to about 6 mm, about 5.2 mm to about 6.5 mm, about 5.2 mm to about 7 mm, about 5.3 mm to about 5.4 mm, about 5.3 mm to about 5.5 mm, about 5.3 mm to about 5.8 mm, about 5.3 mm to about 6 mm, about 5.3 mm to about 6.5 mm, about 5.3 mm to about 7 mm, about 5.4 mm to about 5.5 mm, about 5.4 mm to about 5.8 mm, about 5.4 mm to about 6 mm, about 5.4 mm to about 6.5 mm, about 5.4 mm to about 7 mm, about 5.5 mm to about 5.8 mm, about 5.5 mm to about 6 mm, about 5.5 mm to about 6.5 mm, about 5.5 mm to about 7 mm, about 5.8 mm to about 6 mm, about 5.8 mm to about 6.5 mm, about 5.8 mm to about 7 mm, about 6 mm to about 6.5 mm, about 6 mm to about 7 mm, or about 6.5 mm to about 7 mm. In some cases, the length 126 of the proximal one or more insulators 108 may comprise about 5 mm, about 5.1 mm, about 5.2 mm, about 5.3 mm, about 5.4 mm, about 5.5 mm, about 5.8 mm, about 6 mm, about 6.5 mm, or about 7 mm. In some cases, the length 126 of the proximal one or more insulators 108 may comprise at least about 5 mm, about 5.1 mm, about 5.2 mm, about 5.3 mm, about 5.4 mm, about 5.5 mm, about 5.8 mm, about 6 mm, or about 6.5 mm. In some cases, the length 126 of the proximal one or more insulators 108 may comprise at most about 5.1 mm, about 5.2 mm, about 5.3 mm, about 5.4 mm, about 5.5 mm, about 5.8 mm, about 6 mm, about 6.5 mm, or about 7 mm.

In some cases, the length 124 of the distal one or more electrodes 110 may comprise a length of about 2.5 mm to about 4 mm. In some cases, the length 124 of the distal one or more electrodes 110 may comprise a length of about 2.5 mm to about 2.6 mm, about 2.5 mm to about 2.7 mm, about 2.5 mm to about 2.8 mm, about 2.5 mm to about 2.9 mm, about 2.5 mm to about 3 mm, about 2.5 mm to about 3.1 mm, about 2.5 mm to about 3.5 mm, about 2.5 mm to about 4 mm, about 2.6 mm to about 2.7 mm, about 2.6 mm to about 2.8 mm, about 2.6 mm to about 2.9 mm, about 2.6 mm to about 3 mm, about 2.6 mm to about 3.1 mm, about 2.6 mm to about 3.5 mm, about 2.6 mm to about 4 mm, about 2.7 mm to about 2.8 mm, about 2.7 mm to about 2.9 mm, about 2.7 mm to about 3 mm, about 2.7 mm to about 3.1 mm, about 2.7 mm to about 3.5 mm, about 2.7 mm to about 4 mm, about 2.8 mm to about 2.9 mm, about 2.8 mm to about 3 mm, about 2.8 mm to about 3.1 mm, about 2.8 mm to about 3.5 mm, about 2.8 mm to about 4 mm, about 2.9 mm to about 3 mm, about 2.9 mm to about 3.1 mm, about 2.9 mm to about 3.5 mm, about 2.9 mm to about 4 mm, about 3 mm to about 3.1 mm, about 3 mm to about 3.5 mm, about 3 mm to about 4 mm, about 3.1 mm to about 3.5 mm, about 3.1 mm to about 4 mm, or about 3.5 mm to about 4 mm. In some cases, the length 124 of the distal one or more electrodes 110 may comprise a length of about 2.5 mm, about 2.6 mm, about 2.7 mm, about 2.8 mm, about 2.9 mm, about 3 mm, about 3.1 mm, about 3.5 mm, or about 4 mm. In some cases, the length 124 of the distal one or more electrodes 110 may comprise a length of at least about 2.5 mm, about 2.6 mm, about 2.7 mm, about 2.8 mm, about 2.9 mm, about 3 mm, about 3.1 mm, or about 3.5 mm. In some cases, the length 124 of the distal one or more electrodes 110 may comprise a length of at most about 2.6 mm, about 2.7 mm, about 2.8 mm, about 2.9 mm, about 3 mm, about 3.1 mm, about 3.5 mm, or about 4 mm. In some cases, the first electrode of the one or more distal electrodes 108 may be spaced a distance of at least about 1.5 mm from the distal most portion of the elongated body 116.

In some cases, the length 122 of the distal one or more insulators 123 may comprise a length of about 1.2 mm to about 3 mm. In some cases, the length 122 of the distal one or more insulators 123 may comprise a length of about 1.2 mm to about 1.3 mm, about 1.2 mm to about 1.4 mm, about 1.2 mm to about 1.5 mm, about 1.2 mm to about 1.6 mm, about 1.2 mm to about 1.7 mm, about 1.2 mm to about 1.8 mm, about 1.2 mm to about 1.9 mm, about 1.2 mm to about 2 mm, about 1.2 mm to about 2.5 mm, about 1.2 mm to about 3 mm, about 1.3 mm to about 1.4 mm, about 1.3 mm to about 1.5 mm, about 1.3 mm to about 1.6 mm, about 1.3 mm to about 1.7 mm, about 1.3 mm to about 1.8 mm, about 1.3 mm to about 1.9 mm, about 1.3 mm to about 2 mm, about 1.3 mm to about 2.5 mm, about 1.3 mm to about 3 mm, about 1.4 mm to about 1.5 mm, about 1.4 mm to about 1.6 mm, about 1.4 mm to about 1.7 mm, about 1.4 mm to about 1.8 mm, about 1.4 mm to about 1.9 mm, about 1.4 mm to about 2 mm, about 1.4 mm to about 2.5 mm, about 1.4 mm to about 3 mm, about 1.5 mm to about 1.6 mm, about 1.5 mm to about 1.7 mm, about 1.5 mm to about 1.8 mm, about 1.5 mm to about 1.9 mm, about 1.5 mm to about 2 mm, about 1.5 mm to about 2.5 mm, about 1.5 mm to about 3 mm, about 1.6 mm to about 1.7 mm, about 1.6 mm to about 1.8 mm, about 1.6 mm to about 1.9 mm, about 1.6 mm to about 2 mm, about 1.6 mm to about 2.5 mm, about 1.6 mm to about 3 mm, about 1.7 mm to about 1.8 mm, about 1.7 mm to about 1.9 mm, about 1.7 mm to about 2 mm, about 1.7 mm to about 2.5 mm, about 1.7 mm to about 3 mm, about 1.8 mm to about 1.9 mm, about 1.8 mm to about 2 mm, about 1.8 mm to about 2.5 mm, about 1.8 mm to about 3 mm, about 1.9 mm to about 2 mm, about 1.9 mm to about 2.5 mm, about 1.9 mm to about 3 mm, about 2 mm to about 2.5 mm, about 2 mm to about 3 mm, or about 2.5 mm to about 3 mm. In some cases, the length 122 of the distal one or more insulators 123 may comprise a length of about 1.2 mm, about 1.3 mm, about 1.4 mm, about 1.5 mm, about 1.6 mm, about 1.7 mm, about 1.8 mm, about 1.9 mm, about 2 mm, about 2.5 mm, or about 3 mm. In some cases, the length 122 of the distal one or more insulators 123 may comprise a length of at least about 1.2 mm, about 1.3 mm, about 1.4 mm, about 1.5 mm, about 1.6 mm, about 1.7 mm, about 1.8 mm, about 1.9 mm, about 2 mm, or about 2.5 mm. In some cases, the length 122 of the distal one or more insulators 123 may comprise a length of at most about 1.3 mm, about 1.4 mm, about 1.5 mm, about 1.6 mm, about 1.7 mm, about 1.8 mm, about 1.9 mm, about 2 mm, about 2.5 mm, or about 3 mm.

Usually, the electrodes may be manufactured by various methods. In some embodiments, the electrode comprises a flexible printed circuit. In some embodiments, the electrode is wrapped around the outer surface of the sheath elongate shaft. In some embodiments, the electrode is bonded to the outer surface of the sheath elongate shaft. In some embodiments, the manufacturing method chosen facilitates a large-scale manufacturing of the electrode in bulk. In some embodiments, the manufacturing method chosen facilitates accurate manufacturing of the electrode with low tolerances.

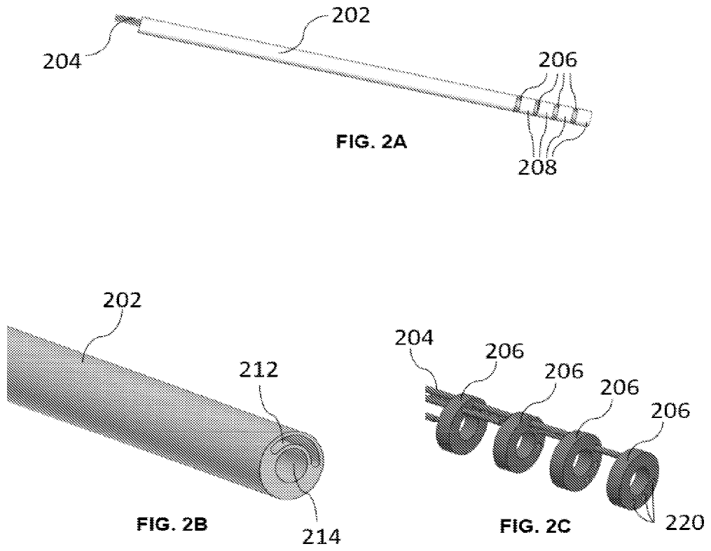

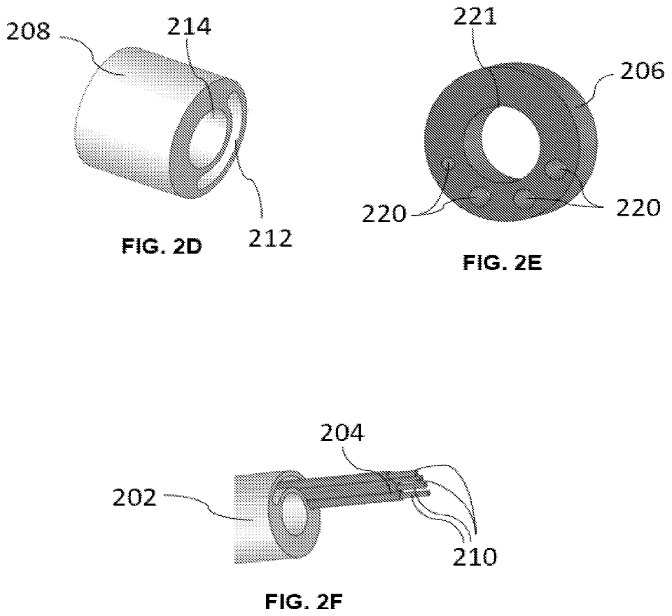

In some embodiments, the one or more electrodes (120, 110) may comprise one or more stacked ring electrodes 206, as shown in FIGS. 2B-2F. In some embodiments, the electrodes may comprise one or more stacked electrodes 206 and one or more isolator rings having the electrodes electrically connected to the cores of a ribbon cable 204. In some cases, the ribbon cable 204 may be in electrical communication with one or more stacked electrodes 206 through an electrical coupling feature 220 of the one or more stacked ring electrodes 206, shown in FIG. 2C and 2E. In some instances, the one or more stacked electrodes 206 may comprise a central lumen 221 configured to align with the central lumen of the sheath 214 and/or the one or more insulators 208.

In some instances, the ribbon cable 204 may comprise a plurality of electrical conductors 210. In some cases, the plurality of electrical conductors 210 may be coated and/or covered by an electrically insulating material configured to prevent flow of electricity and/or electrical coupling. In some cases, the ribbon cable plurality of electrical conductors 210 may each individually connect one or more of the stacked electrodes 206, as seen in FIG. 2A. In some instances, the electrical coupling features 220 may comprise an inner diameter configured to be in a slip mechanical fit with the cover and/or insulated one or more conductors 210 of the ribbon cable. In some instances, such electrical wiring may provide spatial control of sensing or delivering electrical signals. In some cases, one or more insulators 208 may be placed adjacent to one or more stacked ring electrodes 206, as seen in FIG. 2A.

In some embodiments, the ribbon cable 204 may pass through a slot 212 in the wall of the sheath 202 or could lie on its surface or in a shallow surface slot. In some embodiments, the bonding of stacked insulators/conductors is sufficient to withstand the insertion forces. In some embodiments, the insulator 208 comprises at least one slot 212 configured to fit the ribbon cables 204 and a central lumen 214.

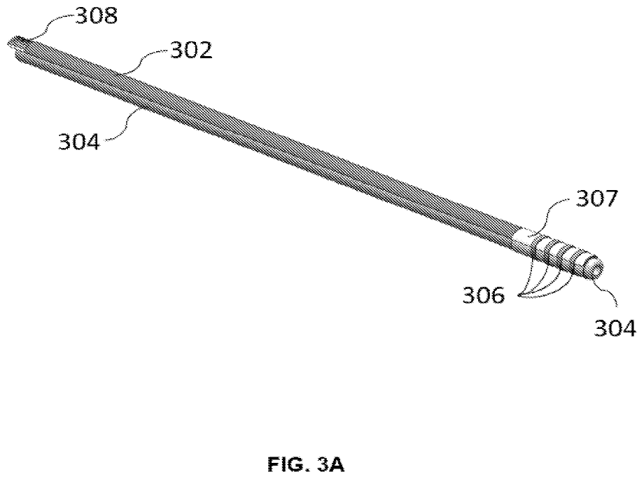

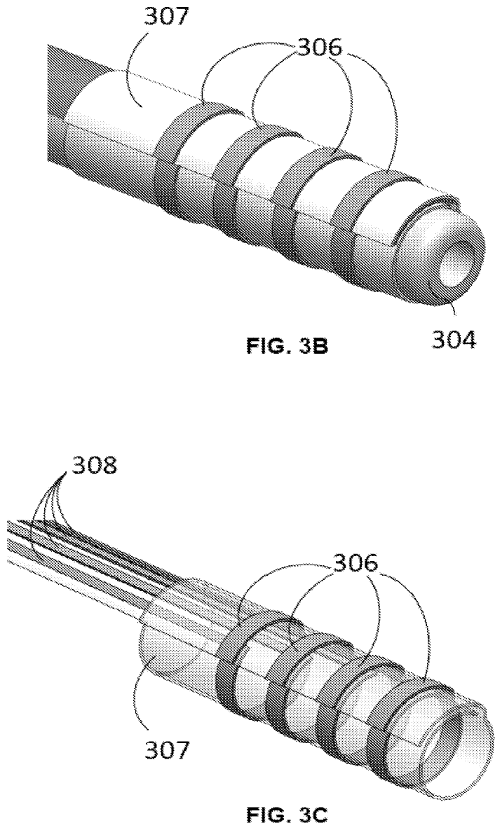

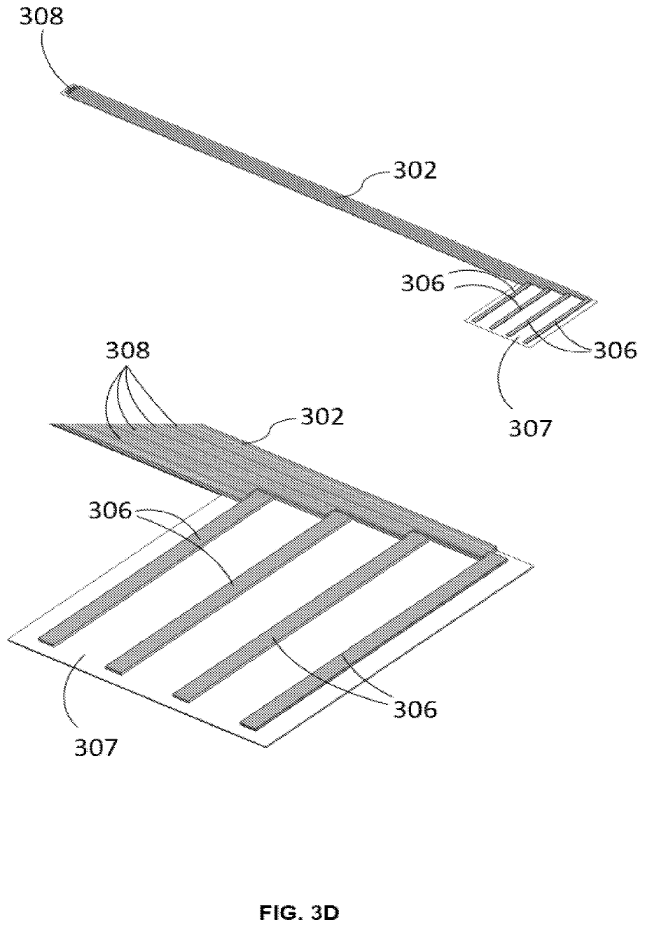

In some embodiments, the one or more electrodes (120, 110) may comprise one or more flexible printed circuit electrodes, as seen in FIGS. 3A-3D. In some embodiments, the one or more electrodes 306 may be wrapped around the outer surface of the sheath elongate shaft 304. In some embodiments, the electrodes 306 may be prepared as a laminated film 304, 302 comprising conductive tracks 308. In some embodiments, the laminated film may wrap around and bonded to the outside of the sheath elongate shaft. In some embodiments, the sheath elongate shaft 304 may be recessed for the film thickness. In some embodiments, conductive tracks may be covered with insulator 302. In some embodiments, the film is in a flat form before being wrapped around the sheath elongate shaft. In some cases, the film may comprise one or more insulator regions 307, as seen in FIGS. 3A-3D. In some cases, the electrodes 306 may be on and/or around the one or more insulator regions 307 and electrically and in communication with the conductive tracks 302.

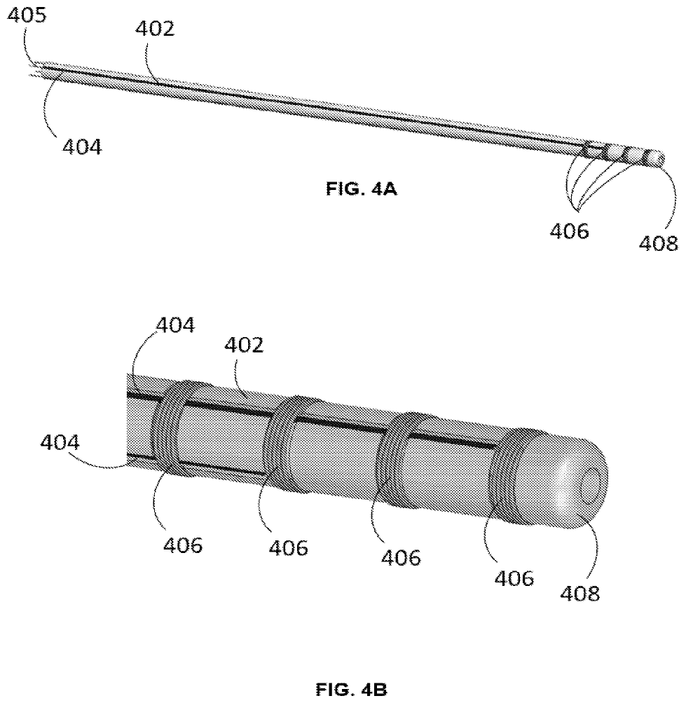

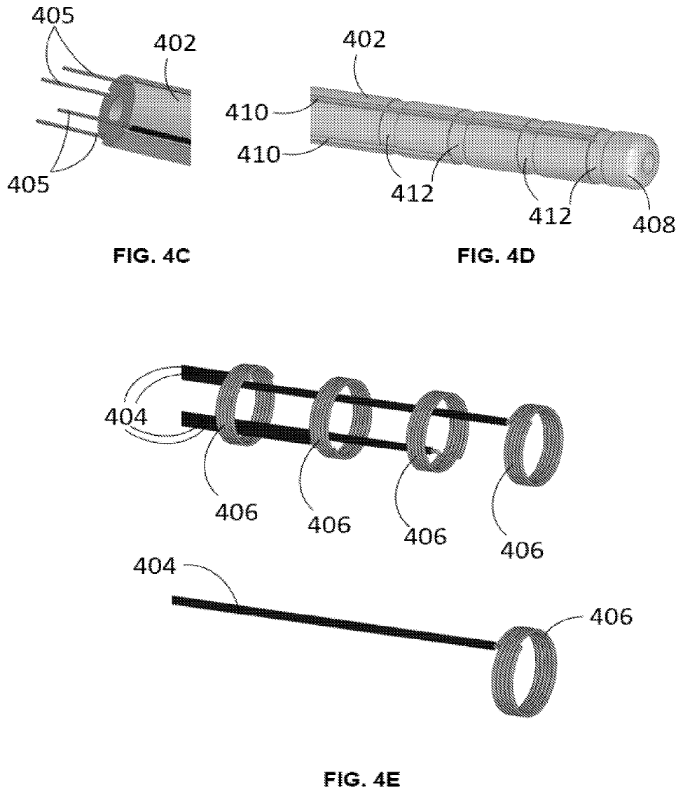

In some embodiments, the one or more electrodes (120, 110) may comprise one or more coil electrodes, as seen in FIGS. 4A-4E. In some embodiments, the one or more coil electrodes 406 may be wrapped around the outer surface of the sheath elongated shaft 408. In some embodiments, the one or more coil electrodes may be manufactured by stripping off an insulative coating and/or cover off and the solid core wire 404, thereby exposing the inner conductor, and winding the inner conductor to form a coil electrode. The coil electrodes may be bonded to the sheath elongated shaft 408 by an adhesive, tension forces, press fit, or any combination thereof coupling. In some embodiments, the one or more coil electrodes 406 may be connected to a solid core wire 404 with insulation. In some embodiments, the solid core wire 404 with insulation may be stripped such that the solid core wires 404 may expose an electric conducting core 405 that may be connected to the one or more coil electrodes 406. In some embodiments, the insulation of the solid core wire 404 may prevent a solid core wire 404 from electrically coupling to the one or more electrical coil electrodes 406 even when the solid core wires 404 are adjacent to the one or more electrical coils, as seen in FIG. 4B. In some embodiments, the sheath elongated shaft 408 may be recessed 410, 412 for the electrodes and wires. In some instances, the recessed features 410 and 412 may establish nonconductive regions of the sheath elongated shaft 402. In some instances, the solid core wires 404 may be terminated at the distal region of the sheath elongated shaft 402, as seen in FIG. 4C, where the solid core wires 404 may interface with an electrical coupling interface in electrical communication with the system providing and/or sensing electrical signals.

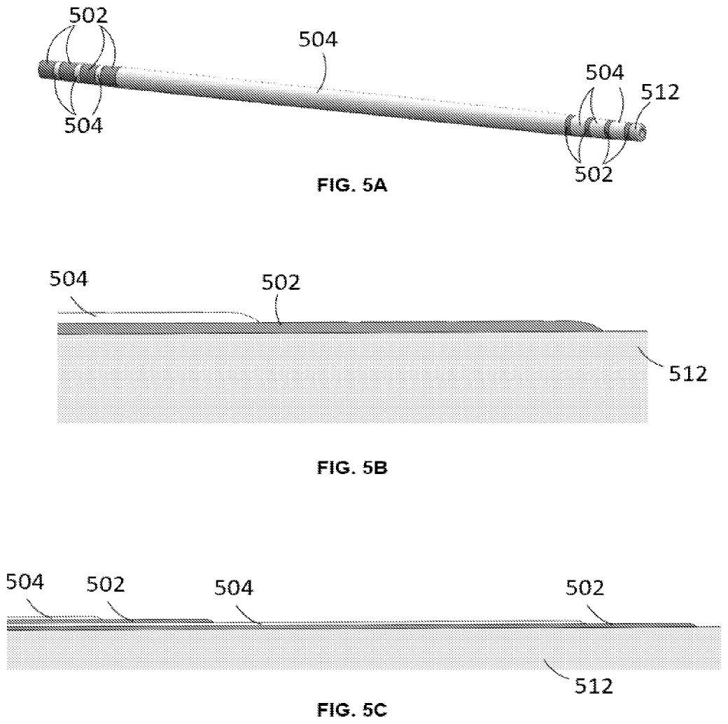

In some embodiments, the one or more electrodes (120, 110) may be bonded to the outer surface of the elongated body 116, as shown in FIGS. 5A-5C. In some embodiments the one or more electrodes 502 may be deposited adjacent to the surface of the elongated body 116 through a dip coating processing. For example, the elongated body 512 may be repeatedly dipped (FIG. 5C) in conductive ink and/or insulating non-conductive material to controlled depths to create annular electrodes 502 and insulating layers 504. In some embodiments, the elongated body 512 may be dipped in alternating conductive ink and/or insulating material to generate one or more annular electrode 502

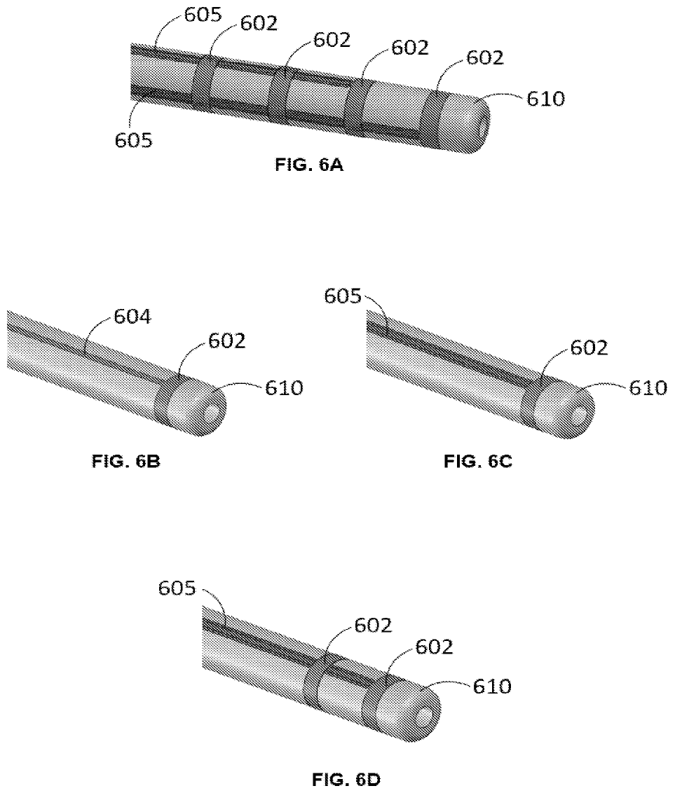

In some embodiments, the one or more electrodes (120, 110) may be printed adjacent to the outer surface of the sheath elongated body 610, as shown in FIGS. 6A-6D. In some embodiments, the electrodes 602 may be printed as conductive tracks covered with insulating layer 605. In some embodiments, the printing may comprise inkjet printing, three-dimensional printing, multi-axis three-dimensional printing, or any combination thereof. In some embodiments, there may be a buildup of either conductive track electrodes 602 or insulating layers 605 by repeated applications of conductive and insulating ink. In some cases, one electrode 602 and electrode lead conductor 604 of the one or more printed electrodes may be printed at a first time point followed by printing an insulating layer 605 at a second time point after the first time point, as seen in FIG. 6B-6C. In some cases, at a third time point after the second time point, a second electrode 602 may then be printed over a region of insulated conductor 605, as seen in FIG. 6D. This process may be repeated one or more times to generate one or more electrodes 602. In some cases, the electrode lead conductors 604 may be printed such that electrical signals may be delivered and/or sensed from the distal region of the sheath elongated body 610 to the distal region of the sheath elongated body 610 where the electrodes may be located.

In some embodiments, the electrodes are designed to deliver various amounts of voltage, current, and/or power. In some embodiments, the electrodes are designed to deliver a voltage of about 10V per electrode. In some embodiments, the electrodes are designed to deliver a voltage of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 V per electrode. In some embodiments, the electrodes are designed to deliver a voltage of at most about 5, 10, 15, 20, 25, 30, 35, 40, 45, or 50 V per electrode. In some embodiments, the electrodes are designed to deliver a voltage of about 1 to about 50 V per electrode, about 1 to about 40 V per electrode, about 1 to about 30 V per electrode, or about 1 to about 20 V per electrode. In some embodiments, the electrodes are designed to deliver a current of about 10 mA per electrode. In some embodiments, the electrodes are designed to deliver a current of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mA per electrode. In some embodiments, the electrodes are designed to deliver a current of at most about 5, 10, 15, 20, 25, 30, 35, 40, 45, or 50 mA per electrode. In some embodiments, the electrodes are designed to deliver a current of about 1 to about 50 mA per electrode, about 1 to about 40 mA per electrode, about 1 to about 30 mA per electrode, or about 1 to about 20 mA per electrode. In some embodiments, the electrodes are designed to deliver a power (VA) of about 0.1 W per electrode. In some embodiments, the electrodes are designed to deliver a power (VA) of at least about 0.01, 0.05, 0.1, 0.5, 1, 2, 3, 4, or 5 W per electrode. In some embodiments, the electrodes are designed to deliver a power (VA) of at most about 0.05, 0.1, 0.5, 1, 2, 3, 4, 5, 6, 7, 8, 9, or 10 W per electrode. In some embodiments, the electrodes are designed to deliver a power (VA) of about 0.01 to about 10 W per electrode, about 0.01 to about 5 W per electrode, or about 0.01 to about 1 W per electrode.

Provided herein are devices for placing an electrode lead to a pudendal nerve, the device comprising: an introducer sheath comprising a sheath elongate shaft having a lumen and a sheath handle at a distal end of the sheath shaft, wherein the introducer sheath is configured to carry an electrode on an outer surface of the sheath elongate shaft; an obturator comprising an obturator elongate shaft having a lumen and an obturator handle at a distal end of the obturator elongate shaft, wherein the obturator elongate shaft is configured to fit inside the sheath lumen; and a needle comprising a needle elongate shaft having a lumen, a needle handle at a distal end of the needle elongate shaft, and a needle tip at the proximal end of the needle elongate shaft, wherein the needle elongate shaft is configured to fit inside the obturator lumen, and wherein the lumen of the needle elongate shaft is configured to allow the electrode lead to pass through; wherein the introducer sheath has a stiffness sufficient to guide the placement of the electrode lead at the target site. In some embodiments, the stiffness of the sheath and the needle together is sufficient to allow secure, accurate placement of the electrode lead onto the pudendal nerve. In some embodiments, the stiffness of the sheath is sufficient to allow secure, accurate placement of the electrode lead onto the pudendal nerve.

Described herein are devices for placing an electrode lead to a pudendal nerve, the device comprising: an introducer sheath comprising a sheath elongate shaft having a lumen and a sheath handle at a distal end of the sheath shaft, wherein the introducer sheath is configured to carry an electrode on an outer surface of the sheath elongate shaft; an obturator comprising an obturator elongate shaft having a lumen and an obturator handle at a distal end of the obturator elongate shaft, wherein the obturator elongate shaft is configured to fit inside the sheath lumen; and a needle comprising a needle elongate shaft having a lumen, a needle handle at a distal end of the needle elongate shaft, and a needle tip at the proximal end of the needle elongate shaft, wherein the needle elongate shaft is configured to fit inside the obturator lumen, and wherein the lumen of the needle elongate shaft is configured to allow the electrode lead to pass through; wherein the introducer sheath has a stiffness sufficient to guide the placement of the electrode lead at the target site. In some embodiments, the needle is removable from the introducer sheath. In some embodiments, the insertable length of the sheath is about 10 cm to about 20 cm. In some embodiments, the introducer sheath has an outer diameter of about 1 mm to about 5 mm. In some embodiments, the introducer sheath has an inner diameter of about 1 mm to about 3 mm. In some embodiments, the introducer sheath has an inner diameter sufficient for the obturator and the needle to pass through. In some embodiments, the inner diameter of the needle shaft is sufficient for the guidewire to pass through. In some embodiments, the needle tip is blunt and has a lumen. In some embodiments, the needle tip extends about 1 mm to about 5 mm beyond the end of the sheath elongate shaft. In some embodiments, the stiffness of the sheath and the needle together is sufficient to allow secure, accurate placement of the electrode lead onto the pudendal nerve. In some embodiments, the stiffness of the sheath is sufficient to allow secure, accurate placement of the electrode lead onto the pudendal nerve. In some embodiments, the stiffness of the sheath is higher than stiffness of sheath for sacral nerve lead placement.

Described herein are devices for placing an electrode lead to a pudendal nerve, the device comprising: an introducer sheath comprising a sheath elongate shaft having a lumen and a sheath handle at a distal end of the sheath shaft, wherein the introducer sheath is configured to carry an electrode on an outer surface of the sheath elongate shaft; an obturator comprising an obturator elongate shaft having a lumen and an obturator handle at a distal end of the obturator elongate shaft, wherein the obturator elongate shaft is configured to fit inside the sheath lumen; and a needle comprising a needle elongate shaft having a lumen, a needle handle at a distal end of the needle elongate shaft, and a needle tip at the proximal end of the needle elongate shaft, wherein the needle elongate shaft is configured to fit inside the obturator lumen, and wherein the lumen of the needle elongate shaft is configured to allow the electrode lead to pass through; wherein the introducer sheath has a stiffness sufficient to guide the placement of the electrode lead at the target site. In some embodiments, the needle is removable from the introducer sheath. In some embodiments, the insertable length of the sheath is about 10 cm to about 20 cm. In some embodiments, the introducer sheath has an outer diameter of about 1 mm to about 5 mm. In some embodiments, the introducer sheath has an inner diameter of about 1 mm to about 3 mm. In some embodiments, the introducer sheath has an inner diameter sufficient for the obturator and the needle to pass through. In some embodiments, the inner diameter of the needle shaft is sufficient for the guidewire to pass through. In some embodiments, the needle tip is blunt and has a lumen. In some embodiments, the needle tip extends about 1 mm to about 5 mm beyond the end of the sheath elongate shaft. In some embodiments, the sheath comprises a plurality of electrically isolated electrodes. In some embodiments, the plurality of electrodes forms about a plurality of wide bands around the sheath with a gap between electrodes and the first band about at least 1 mm from the end of the sheath elongate shaft. In some embodiments, the stiffness of the sheath and the needle together is sufficient to allow secure, accurate placement of the electrode lead onto the pudendal nerve. In some embodiments, the stiffness of the sheath is sufficient to allow secure, accurate placement of the electrode lead onto the pudendal nerve. In some embodiments, the stiffness of the sheath is higher than stiffness of sheath for sacral nerve lead placement. In some embodiments, the electrodes are configured to deliver a voltage of about 5V to about 15V per electrode. In some embodiments, the electrodes are configured to deliver a current of about 5 mA to about 15 mA per electrode. In some embodiments, the electrodes are configured to deliver a power of about 0.05 W to about 0.5 W per electrode.

Provided herein are devices for placing an electrode lead to a pudendal nerve, the device comprising: an introducer sheath comprising a sheath elongate shaft having a lumen and a sheath handle at a distal end of the sheath shaft, wherein the introducer sheath is configured to carry an electrode on an outer surface of the sheath elongate shaft; an obturator comprising an obturator elongate shaft having a lumen and an obturator handle at a distal end of the obturator elongate shaft, wherein the obturator elongate shaft is configured to fit inside the sheath lumen; and a needle comprising a needle elongate shaft having a lumen, a needle handle at a distal end of the needle elongate shaft, and a needle tip at the proximal end of the needle elongate shaft, wherein the needle elongate shaft is configured to fit inside the obturator lumen, and wherein the lumen of the needle elongate shaft is configured to allow the electrode lead to pass through; wherein the introducer sheath has a stiffness sufficient to guide the placement of the electrode lead at the target site. In some embodiments, the needle is removable from the introducer sheath. In some embodiments, the insertable length of the sheath is about 15 cm. In some embodiments, the introducer sheath has a maximum outer diameter of about 5 mm. In some embodiments, the introducer sheath has an inner diameter of about 1 mm to about 2 mm. In some embodiments, the introducer sheath has an inner diameter sufficient for the obturator and the needle to pass through. In some embodiments, the inner diameter of the needle shaft is sufficient for the guidewire to pass through. In some embodiments, the needle tip is blunt and has a lumen. In some embodiments, the needle tip extends a maximum of 3 mm beyond the end of the sheath elongate shaft. In some embodiments, the sheath comprises four electrically isolated electrodes forming about 1.5 mm wide bands around the sheath with a gap between electrodes of about 5 mm and the first band about 1.5 mm from the end of the sheath elongate shaft. In some embodiments, the stiffness of the sheath and the needle together is sufficient to allow secure, accurate placement of the electrode lead onto the pudendal nerve. In some embodiments, the stiffness of the sheath is sufficient to allow secure, accurate placement of the electrode lead onto the pudendal nerve. In some embodiments, the stiffness of the sheath is higher than stiffness of sheath for sacral nerve lead placement. In some embodiments, the electrodes are configured to deliver a voltage of about 10V per electrode. In some embodiments, the electrodes are configured to deliver a current of about 10 mA per electrode. In some embodiments, the electrodes are configured to deliver a power of about 0.1 W per electrode.

Described herein are devices for placing an electrode lead to a pudendal nerve, the device comprising: an introducer sheath comprising a sheath elongate shaft having a lumen and a sheath handle at a distal end of the sheath shaft, wherein the introducer sheath is configured to carry an electrode on an outer surface of the sheath elongate shaft; an obturator comprising an obturator elongate shaft having a lumen and an obturator handle at a distal end of the obturator elongate shaft, wherein the obturator elongate shaft is configured to fit inside the sheath lumen; and a needle comprising a needle elongate shaft having a lumen, a needle handle at a distal end of the needle elongate shaft, and a needle tip at the proximal end of the needle elongate shaft, wherein the needle elongate shaft is configured to fit inside the obturator lumen, and wherein the lumen of the needle elongate shaft is configured to allow the electrode lead to pass through; wherein the introducer sheath has a stiffness sufficient to guide the placement of the electrode lead at the target site. In some embodiments, the needle tip is blunt and has a lumen. In some embodiments, the needle tip is configured to protrude between about 1 mm to 5 mm beyond the end of the obturator lumen. In some embodiments, the needle tip is configured to be retractable into the obturator lumen. In some embodiments, the needle tip angle is configured to advance the needle tip through tissue. In some embodiments, the angle of the proximal end of the sheath elongate shaft allows for advancing the device with little to no damage to surrounding tissue. In some embodiments, the electrode is wrapped around the outer surface of the sheath elongate shaft.

Provided herein are devices for placing an electrode lead to a pudendal nerve, the device comprising: an introducer sheath comprising a sheath elongate shaft having a lumen and a sheath handle at a distal end of the sheath shaft, wherein the introducer sheath is configured to carry an electrode on an outer surface of the sheath elongate shaft; an obturator comprising an obturator elongate shaft having a lumen and an obturator handle at a distal end of the obturator elongate shaft, wherein the obturator elongate shaft is configured to fit inside the sheath lumen; and a needle comprising a needle elongate shaft having a lumen, a needle handle at a distal end of the needle elongate shaft, and a needle tip at the proximal end of the needle elongate shaft, wherein the needle elongate shaft is configured to fit inside the obturator lumen, and wherein the lumen of the needle elongate shaft is configured to allow the electrode lead to pass through; wherein the introducer sheath has a stiffness sufficient to guide the placement of the electrode lead at the target site. In some embodiments, the needle tip is blunt and has a lumen. In some embodiments, the needle tip is configured to protrude between about 1 mm to 5 mm beyond the end of the obturator lumen. In some embodiments, the needle tip is configured to be retractable into the obturator lumen. In some embodiments, the needle tip angle is configured to advance the needle tip through tissue. In some embodiments, the angle of the proximal end of the sheath elongate shaft allows for advancing the device with little to no damage to surrounding tissue. In some embodiments, the electrode is wrapped around the outer surface of the sheath elongate shaft.

Described herein are devices for placing an electrode lead to a pudendal nerve, the device comprising: an introducer sheath comprising a sheath elongate shaft having a lumen and a sheath handle at a distal end of the sheath shaft, wherein the introducer sheath is configured to carry an electrode on an outer surface of the sheath elongate shaft; an obturator comprising an obturator elongate shaft having a lumen and an obturator handle at a distal end of the obturator elongate shaft, wherein the obturator elongate shaft is configured to fit inside the sheath lumen; and a needle comprising a needle elongate shaft having a lumen, a needle handle at a distal end of the needle elongate shaft, and a needle tip at the proximal end of the needle elongate shaft, wherein the needle elongate shaft is configured to fit inside the obturator lumen, and wherein the lumen of the needle elongate shaft is configured to allow the electrode lead to pass through; wherein the introducer sheath has a stiffness sufficient to guide the placement of the electrode lead at the target site. In some embodiments, the needle tip is blunt and has a lumen. In some embodiments, the needle tip is configured to protrude between about 1 mm to 5 mm beyond the end of the obturator lumen. In some embodiments, the needle is removable from the introducer sheath. In some embodiments, the insertable length of the sheath is about 10 cm to about 20 cm. In some embodiments, the introducer sheath has an outer diameter of about 1 mm to about 5 mm. In some embodiments, the introducer sheath has an inner diameter of about 1 mm to about 3 mm. In some embodiments, the introducer sheath has an inner diameter sufficient for the obturator and the needle to pass through. In some embodiments, the inner diameter of the needle shaft is sufficient for the guidewire to pass through. In some embodiments, the needle tip is blunt and has a lumen. In some embodiments, the needle tip extends about 1 mm to about 5 mm beyond the end of the sheath elongate shaft. In some embodiments, the sheath comprises a plurality of electrically isolated electrodes. In some embodiments, the plurality of electrodes forms about a plurality of wide bands around the sheath with a gap between electrodes and the first band about at least 1 mm from the end of the sheath elongate shaft. In some embodiments, the needle tip is configured to be retractable into the obturator lumen. In some embodiments, the electrode is wrapped around the outer surface of the sheath elongate shaft. In some embodiments, the needle tip angle is configured to advance the needle tip through tissue. In some embodiments, the angle of the proximal end of the sheath elongate shaft allows for advancing the device with little to no damage to surrounding tissue. In some embodiments, the obturator has a diameter of about 1 mm to about 4 mm. In some embodiments, the introducer sheath has a diameter of about 1 mm to about 5 mm. In some embodiments, the introducer sheath and the needle have a combined Young's modulus sufficient to allow for the device to penetrate a deep surgical plane in an individual. In some embodiments, the deep surgical plane comprises a surgical plane of muscle, fat, or any combination thereof. In some embodiments, the introducer sheath has a Young's modulus of about 10 mega pascal (MPa) to about 10,000 MPa. In some embodiments, the introducer sheath and needle have a combined Young's modulus to sufficient allows a user to place the lead adjacent to the pudendal nerve. In some embodiments, the stiffness of the sheath and the needle together is sufficient to allow secure, accurate placement of the electrode lead onto the pudendal nerve. In some embodiments, the stiffness of the sheath is sufficient to allow secure, accurate placement of the electrode lead onto the pudendal nerve. In some embodiments, the stiffness of the sheath is higher than stiffness of sheath for sacral nerve lead placement. In some embodiments, the electrodes are configured to deliver a voltage of about 5V to about 15V per electrode. In some embodiments, the electrodes are configured to deliver a current of about 5 mA to about 15 mA per electrode. In some embodiments, the electrodes are configured to deliver a power of about 0.05 W to about 0.5 W per electrode. In some embodiments, the needle tip is configured to be retractable into the obturator lumen. In some embodiments, the needle tip angle is configured to advance the needle tip through tissue. In some embodiments, the angle of the proximal end of the sheath elongate shaft allows for advancing the device with little to no damage to surrounding tissue. In some embodiments, the electrode is wrapped around the outer surface of the sheath elongate shaft.

### Electrode Lead Placement

Described herein are methods, devices, and systems for placing at least one electrode lead onto a target area of the pudendal nerve to treat incontinence. In some cases, the methods, devices, and systems provided herein may be used to place at least one electrode lead on a nerve that serve one or more muscles used for urination to treat urinary incontinence. In some cases, the methods, devices, and systems provided herein may be used to place at least one electrode lead on a nerve that serve one or more muscles used for urination to treat fecal incontinence. Often, accessing the pudendal nerve and placing an electrode or an electrode lead with minimal injury to the surrounding tissues may be difficult by the anatomical structure near the pudendal nerve. In some cases, the introducer may allow for access the pudendal nerve by one or more anatomical paths with little damage to surrounding tissues. In some cases, the introducer may access the pudendal nerve by an ischiorectal approach, where the introducer directed to penetrate or pass close to the sacrotuberous ligament and place the lead to the pudendal nerve trunk at a target location proximal to Alcock's canal. In some cases, the introducer may access the pudendal nerve by a low gluteal approach, where the introducer is directed to pass in the space between sacrotuberous ligament and sacrospinous ligament and to pass anteriorly in the ischiorectal fossa below the pelvic floor to place the lead on the anterior branches of the pudendal nerve to stimulate the pudendal nerve and the dorsal genital nerve.

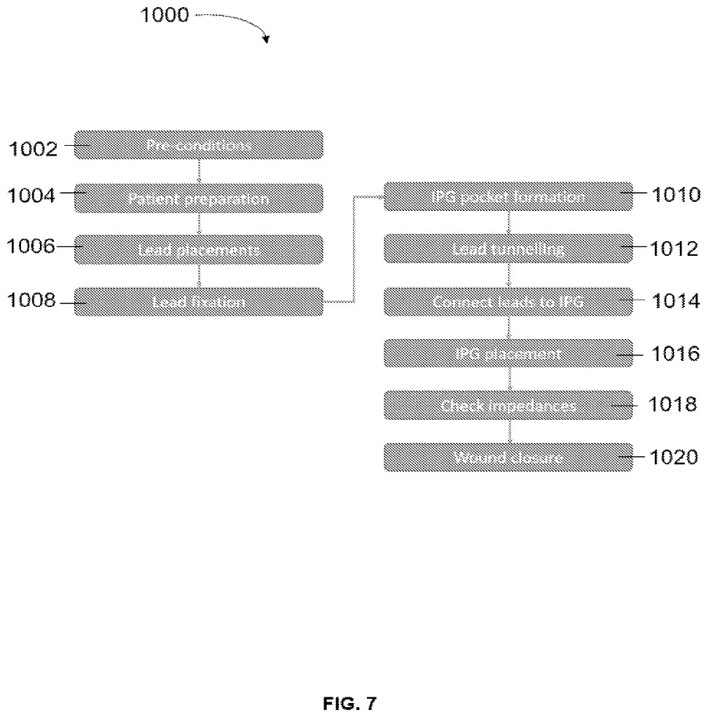

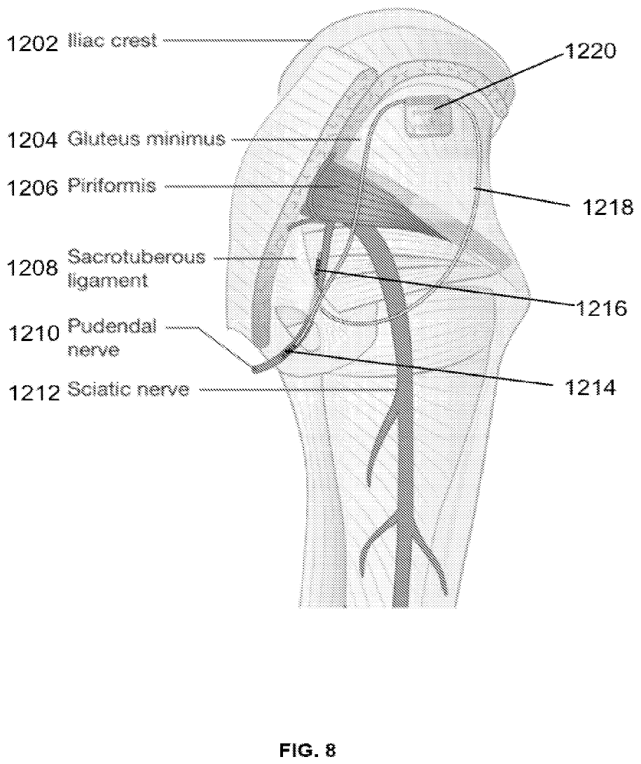

Provided herein are methods, devices, and systems for implanting at least one electrode lead and an implantable pulse generator (IPG) in an individual. Usually, the implantation procedure may involve one or more of steps of pre-condition, patient preparation, placement of one or more leads, fixation of the leads, IPG pocket formation, lead tunneling, connecting the one or more leads to IPG, IPG placement, check of impedances, and tissue closure. FIG. 7 shows an exemplary flowchart 1000 of the steps in for performing electrode lead and implantable pulse generator (IPG) implantation. In some embodiments, the implantation procedure involves sequential steps of pre-condition 1002, patient preparation 1004, placement of one or more leads 1006, fixation of the leads 1008, IPG pocket formation 1010, lead tunneling 1012, connecting the one or more leads to IPG 1014, IPG placement 1016, check of impedances 1018, and wound closure 1020. In some embodiments, the steps are performed by a healthcare professional or a surgeon in an individual to treat incontinence.

Provided herein are methods, devices, and systems to allow for accessing the pudendal nerve by one or more anatomical paths. First, the patient may be positioned in a prone position so as to elevate the buttocks to enable lead insertion by posterior or gluteal approaches. Often, the buttocks may be marked using surface landmarks, including but not limited to greater trochanter and ischial tuberosity, to locate the surface position of the ischial spine. In some cases, using these landmarks and plain radiological imaging (C-arm image intensifier of equivalent), needle electrodes may be inserted to locate the pudendal nerve at each target location. In some cases, the target location comprises distal/pelvic floor via low gluteal approach and proximal trunk of the pudendal nerve via gluteal approach. In some cases, the nerves arc located by intra-operative electrophysiology (EMG responses). In some cases, the nerves are located by visual motor responses. In some cases, the nerves are located by urethral pressure measurements. In some cases, once the nerves are located, the introducer is used to follow the path of the needles to each target location. In some cases, the introducers at the location are advanced and fine alterations are made to their position such that stimulation via a defined proportion of the total number of electrodes leads to a pelvic floor EMG, urethral sphincter, or anal EMG response. In some cases, the fine alterations are made to position of the introducer and the electrodes such that stimulation by at least one of the electrodes leads to a pudendal EMG response. In some cases, the fine alterations are made to position of the introducer and the electrodes such that stimulation by the majority or all of the electrodes leads to a response. In some cases, once the introducers are adequately positioned, the introducer trocars are withdrawn and replaced by the electrode leads using the markings provided to accurately align the lead electrodes with the rings on the introducer. In some cases, the introducers are carefully removed (under image intensification) so as not to disturb lead positioning. In some cases, a small skin incision is made to facilitate access to the leads, which are then fixed in position e.g., by using the fixation devices (threaded onto the lead) and standard non-absorbable monofilament sutures to local fascia. In some cases, the leads are then tunneled to the future IPG site.

In some cases, an electrode lead is placed onto a target area of the target nerve for treating incontinence using the introducer device described herein. In some cases, an electrode lead is placed onto a target area of the target nerve for treating incontinence using a guidewire and/or sheath. In some cases, the guidewire and/or sheath are off-the-shelf components and/or products. In some cases, the target nerve comprises a pudendal nerve. In some embodiments, the electrode needle comprises a Chiba needle. In some embodiments, the lead or guidewire introducer comprises a metal obturator or stiffening wire and an insulating plastic sheath. In some embodiments, the lead or guidewire introducer is advanced over an off-the-shelf guidewire component. In some embodiments, the lead or guidewire introducer is modified to allow easier access to the pudendal nerve. In some embodiments, the lead or guidewire introducer is configured to perforate a ligament. In some embodiments, the lead or guidewire introducer is configured to allow access close to the sacrotuberous ligament. In some embodiments, the sheath of the introducer to access the pudendal nerve has a higher stiffness than a sheath of an introducer typically used to access the sacral nerve.

In some embodiments, the lead comprises a sensor that can obtain neurophysiological recordings from the pudendal nerve. In some embodiments, the method comprises placing a sensor on the pudendal nerve to obtain electrical signal from the pudendal nerve. In some embodiments, the obtained electrical signal may be used to determine the level of neurostimulation of the pudendal nerve to prevent an incontinence episode.

Provided herein are methods for fixing the position of an electrode lead once placed at an targeted location for pudendal nerve stimulation, the method comprising (a) dilating soft tissues deep to the site of skin incision using sharp dissection or an over-sheath device that can be advanced by sliding onto the electrode lead; (b) advancing one or more anchoring devices onto the electrode lead and positioning the one or more anchoring devices into the soft tissue space created by dilatation; and (c) deploying the one or more anchoring devices such that it grips both the electrode lead and the soft tissue space thereby preventing movement of the electrode lead. In some embodiments, the method may apply to both electrode leads and sites of insertion. In some embodiments, the sharp dissection of step (a) of the method may be completed, accomplished and/or achieved using standard surgical instrumentation e.g., a scalpel. In some embodiments, dilating the soft tissues of step (a) of the method may be achieved, completed, and/or accomplished by an over-sheath device to bluntly create a tunnel of a diameter sufficient to permit smooth subsequent advancement of the anchor on the electrode lead. In some embodiments, the soft tissue deep to the site of skin incision may comprise tissue adjacent to the ischial bone and fascial and/or ligamentous insertions. In some embodiments, the one or more anchoring devices may comprise one of a plurality of passive anchors. In some embodiments, advancing the one or more anchoring devices onto the electrode lead may be accomplished, completed, and/or achieved by manually advancing the one or more anchoring devices (e.g., pushing by hand or basic surgical instrument such as a clip) onto the electrode lead. In some embodiments, advancing the one or more anchoring device may be accomplished, completed, and/or achieved using an over-sheath device to advance the one or more anchoring devices into position on the electrode lead. In some embodiments, the one or more anchoring devices may be contained within the over-sheath device before deployment. In some embodiments, the one or more anchoring devices may be secured to the electrode lead with one or more fixation methods. In some embodiments, the one or more anchoring devices may be secured to the soft tissue space by one or more fixation methods. In some embodiments, the one or more anchoring devices may be fixed to the soft tissue space, where the soft tissue space may comprise native human tissues e.g., ligamentous, fascial, periosteal, or any combination thereof tissues. In some embodiments, the one or more anchoring devices may be fixed to the soft tissue space using standard surgical approaches e.g., suturing. In some embodiments, physical features of the one or more anchoring devices may fix and/or secure the one or more anchoring devices to the native human tissues. In some embodiments, the one or more anchoring devices may be fixed to the electrode lead by one or more fixation methods. In some embodiments, the one or more anchoring devices may be fixed to the electrode lead by frictional force exerted between the one or more anchor devices and the electrode lead. In some embodiments, ligatures may be used to compress the one or more anchoring devices onto the electrode lead. In some embodiments, standard suture material may provide ligatures for compression of the one or more anchoring devices onto the electrode lead thereby fixing and/or securing the one or more anchoring devices to the electrode lead. In some embodiments, a small-mounted screw assembly may provide compression of the one or more anchoring devices onto the electrode thereby securing and/or fixing the one or more anchoring device onto the electrode lead. In some cases, the small-mounted screw assembly may be tightened using a miniaturized torque wrench to a pre-specified pressure limit. In some embodiments, miniaturized torque wrench may allow tightening of the small-mounted screw assembly deep in soft tissues. In some embodiments, standard surgical ligating clips may be used to compress the one or more anchoring devices onto the electrode lead thereby securing and/or fixing the one or more anchoring devices onto the electrode lead. In some embodiments, the standard surgical ligating clips may be applied using a ligating clip applier. In some embodiments, the one or more anchoring devices may be fixed to the electrode lead and/or surrounding soft tissues by an activating mechanism, e.g., spring loaded tines, described elsewhere herein. In some embodiments, the common anchoring mechanism of the one or more anchoring devices may be deployed automatically on extrusion of the one or more anchoring devices from the over-sheath device. In some embodiments, retraction of the over-sheath device may activate a common anchoring mechanism of the one or more anchoring devices. In some embodiments, the activation of anchoring and/or fixing of the one or more anchoring devices may be activated when the one or more anchoring devices are pushed out from the over-sheath device. In some embodiments, the one or more anchoring devices may be pushed out of the over-sheath devices by a pushing device. In some embodiments, the one or more anchoring devices may comprise one or more mechanisms that are activated automatically when the one or more anchoring devices are extruded from the over-sheath device. In some embodiments, the one or more mechanisms may comprise activation of spring-loaded tines of the one or more anchoring devices. In some embodiments, the activating mechanism may comprise a clipping function of the one or more anchoring devices to the electrode lead and to the soft tissue space.

### Pudendal Nerve

The pudendal nerve is a major nerve in the pelvic region. Usually, the pudendal nerve may run through the pelvic floor muscles that support organs and ends at external genitalia. Often, the pudendal nerve may send motor and sensation information from the genital area. In some cases, the pudendal nerve may be crucial for sensation and function in the pelvic region. In some cases, the pudendal nerve is a part of the peripheral nervous system.

Typically, the pudendal nerve is found bilaterally, one for each side of the body, on the left and the right. In some cases, the pudendal nerve may arise from the sacral plexus in the lowest part of the spine. In some cases, the sacral plexus comprise a bundle of nerves located on the back of the pelvis. In some cases, the sacral plexus comprises a complex network of nerves that give and receive feedback on movement and sensation to the thighs, lower legs, feet, and part of the pelvis. Usually, the pudendal nerve connects to the S2 to S4 sacral spinal nerve roots in the sacral plexus and runs through the pelvis and gluteal region at the upper end of the femur. Often, the pudendal nerve passes through the greater sciatic foramen, exits the gluteal region through the lesser sciatic foramen, and travels alongside the pudendal artery and vein into the pudendal canal, also referred herein as the Alcock's canal, a narrow tunnel-like opening in the pelvis. In some cases, the pudendal nerve divides into smaller nerve branches after entering the pudendal canal. In some cases, the pudendal nerve branches into inferior rectal nerve, perineal nerve, and dorsal genital nerve. In some cases, the inferior rectal nerve controls the anal sphincter and sends sensory and motor information to the anal sphincter and anal canal. In some cases, the pudendal nerve plays a role in reflex control of bladder contraction and emptying. In some cases, the perineal nerve controls the pelvic floor muscles and the urethral sphincter. In some cases, the perineal nerve provides sensory and motor information from the perineum and the labia or scrotum. In some cases, the dorsal nerve sends sensory information, including but not limited to touch, pleasure, pain, to the skin of penis or clitoris.

In some cases, the motor function of the pudendal nerve controls the movement of one or more muscles. In some cases, the motor function of the pudendal nerve controls the movement one or more of anal sphincter muscles and urethral sphincter muscles. In some cases, the anal sphincter muscles aid in holding in and release of feces. In some cases, urethral sphincter muscles aid in holding in and release of urine. In some cases, the pudendal nerve provides sensory information about touch, pleasure, pain, and temperature of various anatomy, including but not limited to penis, vagina, perineum, anus, and anal canal. In some cases, the pudendal nerve injuries may result in one or more of loss of sensation in the nerve's distribution, fecal and urinary incontinence, sexual dysfunction, or a combination thereof.

### Pre-Conditions

In some embodiments, the individual experiencing incontinence and being prepared for treatment by electrical nerve stimulation may undergo various pre-condition steps before the start of the implantation procedure. In some embodiments, the implantation procedure is performed in a sterile operating room environment with laminar flow or a similar condition. In some embodiments, the sterile operating room environment has limited entry and movement of personnel. In some embodiments, the equipment used for the procedure, including but not limited to the introducer, are sterilized prior to the procedure. In some embodiments, the introducer comprised materials compatible with standard sterilization procedures, including but not limited to ethylene oxide gas, gamma irradiation, and autoclave sterilization. In some embodiments, the surgical table may be capable of various patient positioning and X-ray C-arm access. In some embodiments, a radiographer, also known as radiologic technologist, may be present during the procedure to work with an image intensifier. In some embodiments, one or more non-invasive imaging methods are used along the anatomical path of the introducer during the implantation procedure to provide images of one or more of the anatomy, needle insertions, the introducer, the electrodes, and/or the leads. In some embodiments, the patient controller may be fully charged and linked to the IPG prior to surgery. In some embodiments, the IPG may be linked and charged through its packaging to maintain sterility. In some embodiments, the patient controller may be placed in a sterile bag and linked during surgery.

### Patient Preparation

In some embodiments, the individual may be prepared for treatment by electrical nerve stimulation before the start of the implantation procedure. In some embodiments, the IPG implantation site may be pre-marked in relation to posture and clothing to increase comfort for the individual after the procedure in their daily lives. In some embodiments, the individual may be given a general anesthetic prior to the procedure. In some embodiments, the individual is adequately positioned in prone jack-knife to allow surgical access. In some embodiments, left/right sided tilting and correct position may be checked prior to the procedure. In some embodiments, a urethral transducer may be inserted in the individual to monitor and improve the accuracy of lead placement. In some embodiments, a transducer on urinary catheter may be used to monitor the progress of the lead placement. In some embodiments, a transvaginal probe may be inserted safely into vagina of the individual for the purposes of measuring EMG. In some embodiments, the transvaginal probe may be used to monitor and improve the accuracy of the lead placement. In some embodiments, the transvaginal probe may be used to monitor the progress of the lead placement. In some embodiments, an electrical ground pad may be placed on the individual away from surgical site. In some embodiments, the skin of the individual, including but not limited to vaginal introitus, may be prepared, and draped prior to the procedure to reduce infection and surgical complications. In some embodiments, a needle electrode may be inserted into the external anal sphincter of the individual for the purposes of measuring EMG. In some embodiments, an adhesive surface electrode may be applied onto the peri-anal skin of the individual for the purposes of measuring EMG.

### Lead Placements

Provided herein are methods, devices, and systems to allow for accessing the pudendal nerve by one or more anatomical paths with little damage to tissues surrounding the pudendal nerve. Often, accessing the pudendal nerve and placing one or more leads with minimal injury to the surrounding tissues may be difficult by the anatomical structure near the pudendal nerve. Described herein are imaging-guided markings on skin to guide the anatomical path of the introducer to access the pudendal nerve. In some embodiments, radiological images of the gluteal area of the individual may be taken with a metal guide placed on the skin. In some embodiments, the radiological images may be used to determine the locations of a series of surface markings on the skin in the gluteal region to provide the direction of the anatomical path of the introducer in the individual to access the pudendal nerve. In some embodiments, the radiological images are taken by fluoroscopy. In some embodiments, the radiological images are taken by X-ray. In some embodiments, the introducer may access the pudendal nerve by an ischiorectal approach, where the introducer is directed to penetrate or pass close to the sacrotuberous ligament and place the lead to the pudendal nerve trunk at a target location proximal to Alcock's canal in the region of the ischial spine. In some embodiments, the introducer may access the pudendal nerve by a low gluteal approach, where the introducer is directed to pass in the space between sacrotuberous ligament and sacrospinous ligament and to pass anteriorly in the ischiorectal fossa below the pelvic floor to place the lead on the anterior branches of the pudendal nerve to stimulate the pudendal nerve and the dorsal genital nerve.

In some embodiments, the low gluteal approach uses surface markings on the skin to traverse the gluteal muscles to reach the ischial spine whereupon electrophysiological responses are used to guide further placement. In some embodiments, the ischiorectal approach enters the skin lateral to the anus near the ischial tuberosity and uses transvaginal or transrectal palpation of the ischial spine +/−electrophysiological responses to place the electrode lead. In some embodiments, low gluteal approach and ischiorectal approach to access the pudendal nerve may benefit from guidance by radiological imaging. In some embodiments, it is generally considered that such approaches target the area of the pudendal nerve at or proximal to the region of Alcock's canal i.e., the nerve trunk. In some embodiments, the proximal pudendal nerve trunk has a fascicular anatomy in which its distal branches are represented as individual fascicles or distinct groups of fascicles. In some embodiments, the fascicular anatomy of the pudendal nerve may affect the accuracy of lead placement, where small changes in lead position may favor certain fascicles and thence different motor or afferent effects.

Often, the pudendal nerve may be accessible to electrical stimulation, but the effectiveness of treating incontinence by PNS may be affected by the site of stimulation. In some embodiments, proximal stimulation to the PN trunk (i.e., above Alcock's canal) may provide direct motor stimulation to both urethral and anal sphincters. In some embodiments, proximal stimulation to the PN trunk may lead to some contraction of the pelvic floor/levator ani, based on stimulation being provided proximal to both inferior rectal and perineal nerve branches. In some embodiments, stimulation in the region of Alcock's canal may provide urethral sphincter contraction but less anal sphincter contraction. In some embodiments, the region of Alcock's canal still proximal to the perineal nerve, and some contraction of the pelvic floor/levator ani may be anticipated from stimulation of this region. In some embodiments, unilateral stimulation may lead to bilateral motor effects based on anatomical dissections. In some embodiments, more distal stimulation (i.e., of the dorsal genital nerve) may lead to only effects mediated by afferent stimulation. In some embodiments, the fascicular anatomy of the PN trunk may be important for accuracy of lead placement.

FIGS. 8-11 show the anatomical path desired for lead placements. FIG. 8 shows an exemplary schematic of the anatomy and the disposition of leads and IPG in an individual. FIG. 8 shows iliac crest 1202 of the ileum, gluteus minimus 1204, piriformis 1206, sacrotuberous ligament 1208, pudendal nerve 1210, and sciatic nerve 1212. In some embodiments, the leads 1214, 1216 may be placed on one or more locations along the length of the pudendal nerve 1210. In some embodiments, the wires 1218 of the leads 1214, 1216 may be connected to the IPG 1220. In some embodiments, the placement of the leads 1214, 1216 on the pudendal nerve may be verified and fixed before their wires are connected to the IPG.

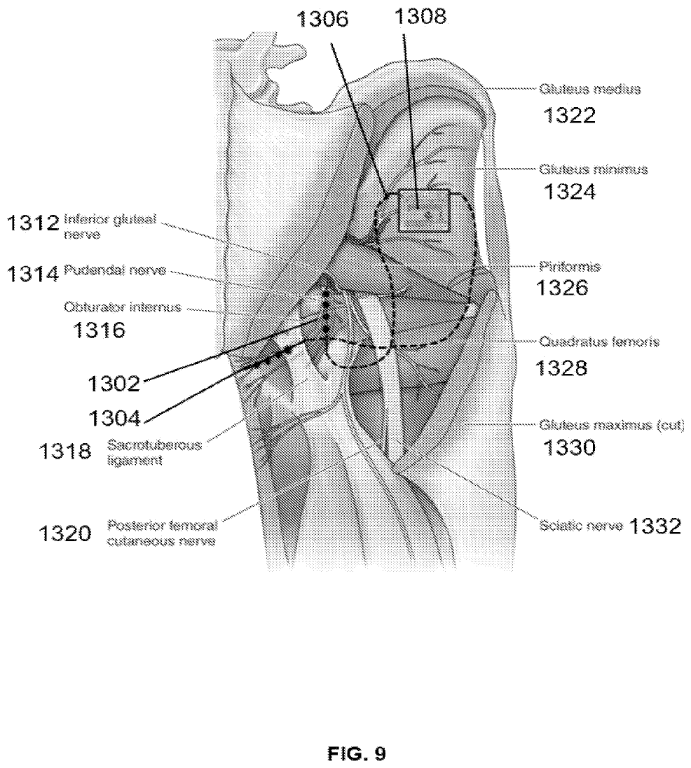

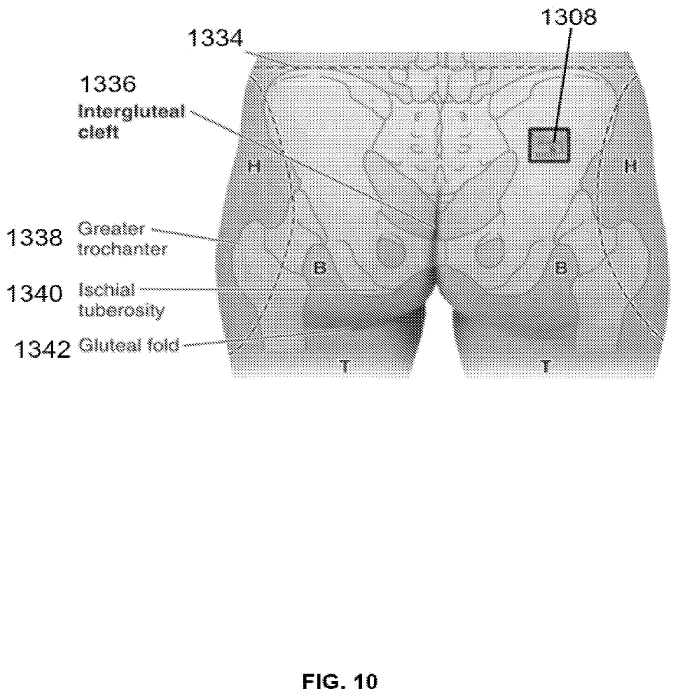

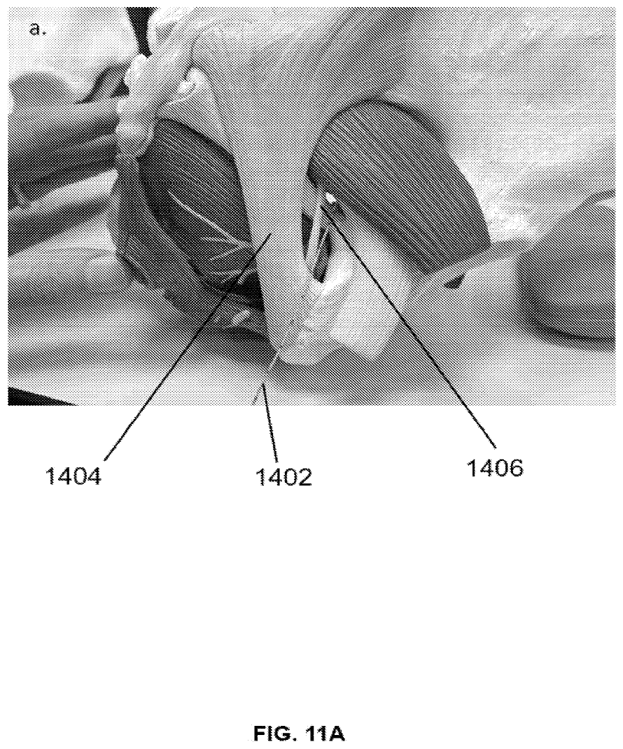

FIGS. 9 and 10 show exemplary schematics of the anatomy and implanted leads and IPG in an individual. FIG. 9 shows two leads 1302, 1304, each lead with four electrodes (shown as dark circles), placed on two sections of the pudendal nerve 1314. In some embodiments, the wires 1306 of the leads 1302 and 1304 may be connected to the IPG 1308. Shown in FIG. 9 are inferior gluteal nerve 1312, pudendal nerve 1314, obturator internus 1316, sacrotuberous ligament 1318, posterior femoral cutaneous nerve 1320, gluteus medius 1322, gluteus minimus 1324, piriformis 1326, quadratus femoris 1328, gluteus maximus 1330, and sciatic nerve 1332. FIG. 10 shows the IPG 1308 placed in a pocket in the buttock fat overlying the gluteal muscles. Shown in FIG. 10 are iliac crest 1334, intergluteal cleft 1336, greater trochanter 1338 of the femur, ischial tuberosity 1340 of the pelvis, and gluteal fold 1342.

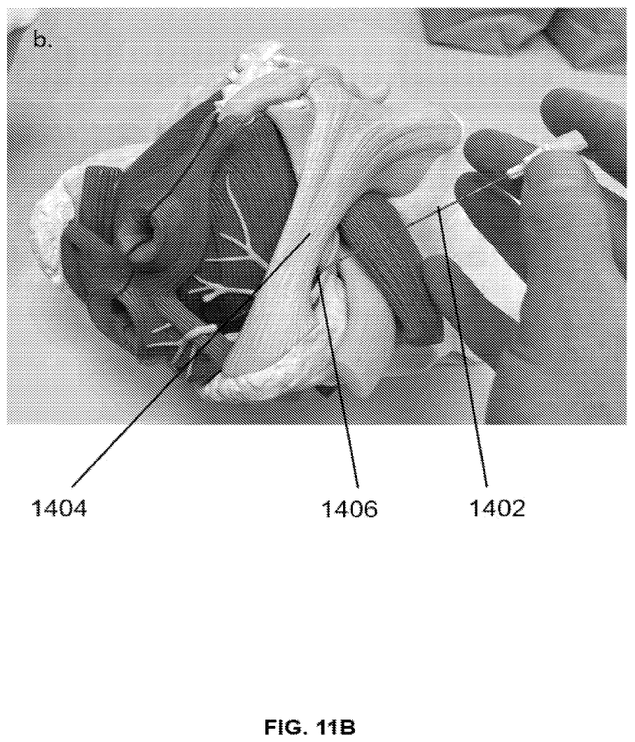

FIGS. 11A and 11B show exemplary embodiments of the anatomical paths of the leads using anatomical models. FIG. 11A shows an exemplary embodiment of the ischiorectal approach, where a needle 1402, representing the needle of the introducer, is shown passing the sacrotuberous ligament 1404 to access the pudendal nerve 1406. In some embodiments, the needle of the introducer may pierce or pass close to the sacrotuberous ligament 1404 in vivo. FIG. 11B shows exemplary embodiment of the low gluteal approach, where the needle 1402 is directed approximating a low gluteal approach to access the pudendal nerve 1406.

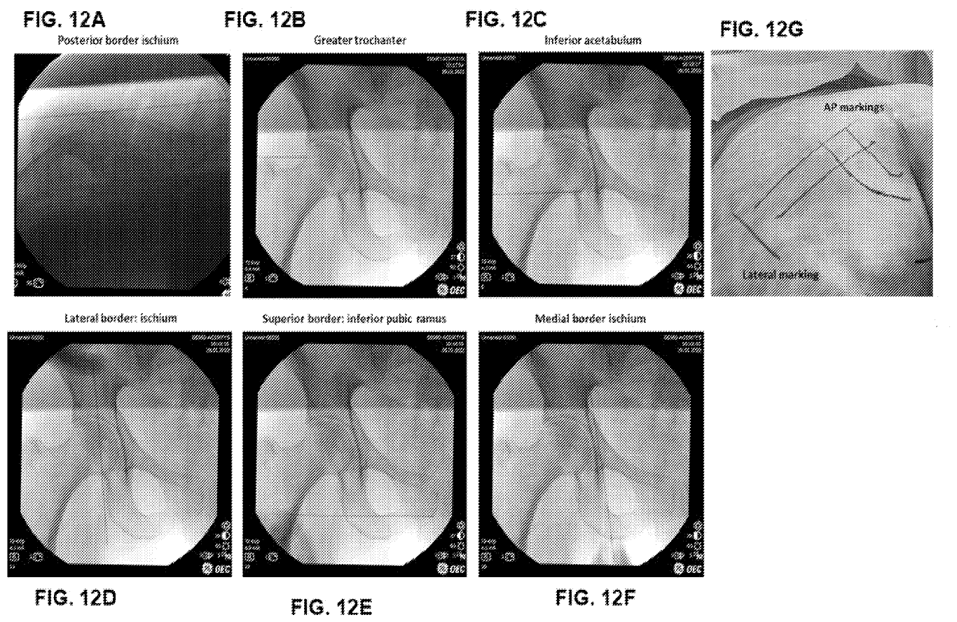

In some embodiments, the lead placement comprises determining the location of a series of surface markings on the skin in the gluteal region from information gathered from radiological images of the gluteal region of the individual. In some embodiments, the radiological images comprise fluoroscopy images. In some embodiments, the radiological images comprise X-ray images. In some embodiments, the radiological images may be used in combination with other non-invasive imaging techniques. In some embodiment, a metal instrument may be used during imaging to provide a guide (e.g., reference) for the surface markings. In some embodiment, the metal instrument may be a straight metal instrument. In some embodiments, the metal instrument may be curved. In some embodiments, the metal instrument may comprise one or more small protrusions or indentations to further aid in its function as a guide. FIGS. 12A-12G show exemplary embodiments of determining and making surface markings using fluoroscopy. FIGS. 12A-12F show fluoroscopy images. In some embodiments, the fluoroscopy images comprise one or more images of posterior border of the ischium (FIG. 12A), greater trochanter (FIG. 12B), inferior border of the acetabulum (FIG. 12C), lateral border of the ischium (FIG. 12D), superior border of the inferior pubic ramus (FIG. 12E), medial border of the ischium (FIG. 12F), or combinations thereof. FIG. 12G shows surface markings (AP markings and lateral markings) in the gluteal region of the individual from information gathered from the fluoroscopy images of FIGS. 12A-12F.

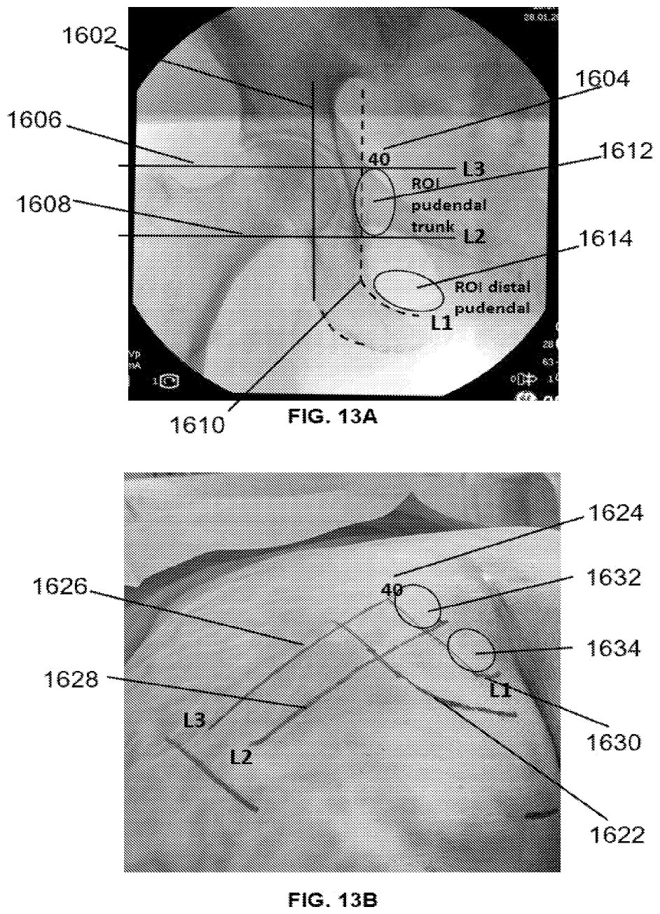

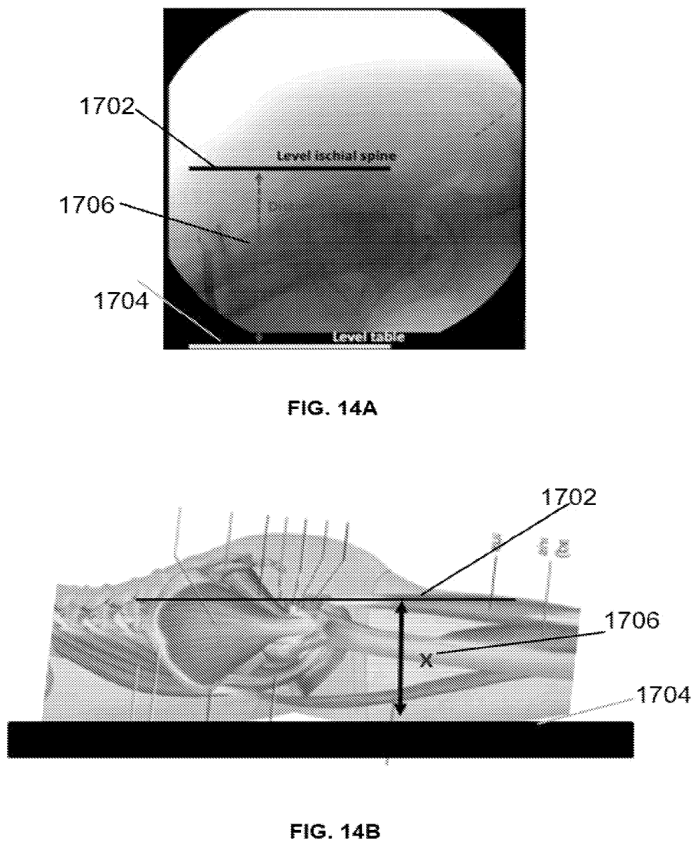

FIGS. 13A-13B show exemplary embodiments of the markings on a radiological image (FIG. 13A) and on skin in the gluteal region (FIG. 13B). In some embodiments, each marking indicates one or more anatomical sites of interest. In some embodiments, the markings comprise at least two lines. In some embodiments, the markings comprise at least three lines. In some embodiments, the markings comprise four lines. In some embodiments, the cross section of the markings may provide an approximate location of a target insertion site for the introducer. In some embodiments, the markings help indicate at least one anatomical region of interest for the implantation procedure. In some embodiments, the anatomical region of interest comprises the location of a portion of the pudendal nerve. In some embodiments, the anatomical region of interest comprises the location of the trunk of the pudendal nerve. In some embodiments, the anatomical region of interest comprises the location of the distal part of the pudendal nerve. In some embodiments, the markings comprise a horizontal line through the superior tip of the greater trochanter of the femur to mark the level of the ischial spine, labeled as L3 in a radiological image 1606 or on the skin of the individual 1626. In some embodiments, the markings comprise a horizontal line through the inferior border of the acetabulum, labeled as L2 in a radiological image 1608 or on the skin of the individual 1628. In some embodiments, the L2 line marks the upper part of the sacrotuberous ligament. In some embodiments, the L2 line may define the lower border of the lesser sciatic foramen, which may indicate the site where the pudendal nerve turns from the gluteal area into the ischiorectal fossa. In some embodiments, the markings comprise a curved line on the inner surface of the ischial bone (inner table of the posterior pelvic ramus), labeled as L1 in a radiological image 1610 or on the skin of the individual 1630. In some embodiments, the markings comprise an intersection of L3 line with a line 1602, 1622, labeled as X in a radiological image 1604 or on the skin of the individual 1624. In some embodiments, the X mark represents the ischial spine. In some embodiments, the markings help indicate at least one anatomical area of interest for the implantation procedure. In some embodiments, the anatomical area of interest comprises the location of a portion of the pudendal nerve. In some embodiments, the location of the trunk of the pudendal nerve is indicated by the area of interest that is in between the L3 line 1606, 1626 and L2 line 1608, 1628. In some embodiments, the location of the trunk of the pudendal nerve is indicated by the area of interest that is inferior to the X mark. In some embodiments, the location of the trunk of the pudendal nerve is indicated by the area of interest that lateral to the L1 line. In some embodiments, the location of the trunk of the pudendal nerve is indicated by an oval area shown as 1612 in a radiological image and 1632 on the skin in FIGS. 13A and 13B. In some embodiments, the location of the distal part of the pudendal nerve is indicated by the area of interest that is inferior to the L2 line 1608, 1628 and lateral to the L1 line 1610, 1630. In some embodiments, the location of the distal part of the pudendal nerve is indicated by an oval area shown as 1614 in a radiological image and 1634 on the skin in FIGS. 13A and 13B.

FIGS. 14A-14B show exemplary embodiments of a fluoroscopy image and a schematic in the lateral view. The lateral depth from the level of the ischial spine to the surgical table may be determined to provide the A-P (anterior-posterior) depth of posterior surface of the ischial bone to guide insertion of the introducer. As shown in FIGS. 14A-14B, the line 1702 marks the level of the ischial spine, the line 1704 marks the level of the surgical table, and the dotted line 1706 indicates the height measured from the table. In some embodiments, this height 1706 is used to determine the point of insertion in the perineum. In some embodiments, the level of the upper (posterior) surface of the ischial bone may be marked by placing radio-opaque instrument against the thigh and measuring the distance (x) from the table to the line. In some embodiments, oblique views may help to determine the angle of needle/lead insertion. The X-ray arm may be rotated until the obturator fossa appears on X-ray as a very shallow oval. The inclination of the X-ray arm may parallel the direction of the distal part of the pudendal nerve. In some cases, placing the electrode according to the inclination of the X-ray may put the lead parallel to the target nerve.

In some embodiments, the electrodes on the leads may provide stimulation to the pudendal nerve to control the movement of the anal sphincter and/or urethral sphincter. In some embodiments, the electrodes on the leads may provide stimulation to the pudendal nerve to alter the sensory information from the pelvic floor muscles and/or external genitalia. In some embodiments, the electrodes on the leads may provide stimulation to the pudendal nerve to alter the sensory information to the pelvic floor muscles and/or external genitalia.

In some embodiments, acute pudendal nerve stimulation may lead to urethral closure and pelvic floor contraction. In some embodiments, acute pudendal nerve stimulation may lead to acute inhibition of bladder contractility. In some embodiments, a wide frequency range (1-100 Hz) may be capable of inducing contractions. In some embodiments, the latency of contraction response to the PNS may be short. In some embodiments, contractions may manifest as increases in intra-urethral pressure. In some embodiments, low frequency stimulation in the range of 2-50 Hz of the pudendal nerve or its branches induces reductions in bladder contractility. In some embodiments, low frequency stimulation may range from about 0.1 Hz to about 30 Hz, about 1 Hz to about 30 Hz, about 1 Hz to about 20 Hz, about 2 Hz to about 15 Hz, about 1 Hz to about 10 Hz, or about 1 Hz to 50 Hz. In some embodiments, low frequency stimulation in the range of about 2-50 Hz of the pudendal nerve or its branches may induce reduction in sphincter contraction. In some embodiments, higher frequencies, such as greater than 50 Hz, may lead to unwanted potentiation of bladder contractions and voiding. In some embodiments, higher frequencies may range from about 10 Hz to about 500 Hz, about 20 Hz to about 500 Hz, about 30 Hz to about 500 Hz, about 50 Hz to about 500 Hz, or about 50 Hz to 100 Hz.

In some embodiments, one or more leads may be placed along the pudendal nerve. In some embodiments, one lead may be placed along the pudendal nerve. In some embodiments, two leads may be placed along the pudendal nerve. In some embodiments, three leads may be placed along the pudendal nerve.

In some embodiments, two leads may be spaced apart along the pudendal nerve. In some embodiments, two leads may be spaced apart at least about 1 cm, 2 cm, 3 cm, 4 cm, 5 cm, 6 cm, 7 cm, 8 cm, 9 cm, or 10 cm along the pudendal nerve. In some embodiments, two leads may be spaced apart no more than about 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm along the pudendal nerve. In some embodiments, two leads may be spaced apart between about 1 cm to about 20 cm, about 1 cm to about 15 cm, or about 1 cm to about 10 cm along the pudendal nerve. In some embodiments, two leads may be spaced apart about 1 cm, 2 cm, 3 cm, 4 cm, 5 cm, 6 cm, 7 cm, 8 cm, 9 cm, or 10 cm along the pudendal nerve.

In some embodiments, the lead comprises one or more electrodes. In some embodiments, the lead comprises at least 1, 2, 3, 4, 5, 6, 7, 8, 9, or 10 electrodes. In some embodiments, the lead comprises no more than 2, 3, 4, 5, 6, 7, 8, 9, or 10 electrodes. In some embodiments, the lead comprises 1, 2, 3, 4, 5, 6, 7, 8, 9, or 10 electrodes. In some embodiments, the electrodes are evenly spaced apart. In some embodiments, the spacing between the electrodes are different. In some embodiments, the dimensions of the electrodes on a lead are same. In some embodiments, the dimensions of the electrodes on a lead are different.

### Ischiorectal Approach

Provided herein are methods, devices, and systems to allow for accessing the pudendal nerve by ischiorectal approach with little damage to tissues surrounding the pudendal nerve. In some embodiments, the lead may be placed on the pudendal nerve by an ischiorectal approach, where the introducer is directed to penetrate or pass close to the sacrotuberous ligament and place the lead to the pudendal nerve trunk at a target location proximal to Alcock's canal.

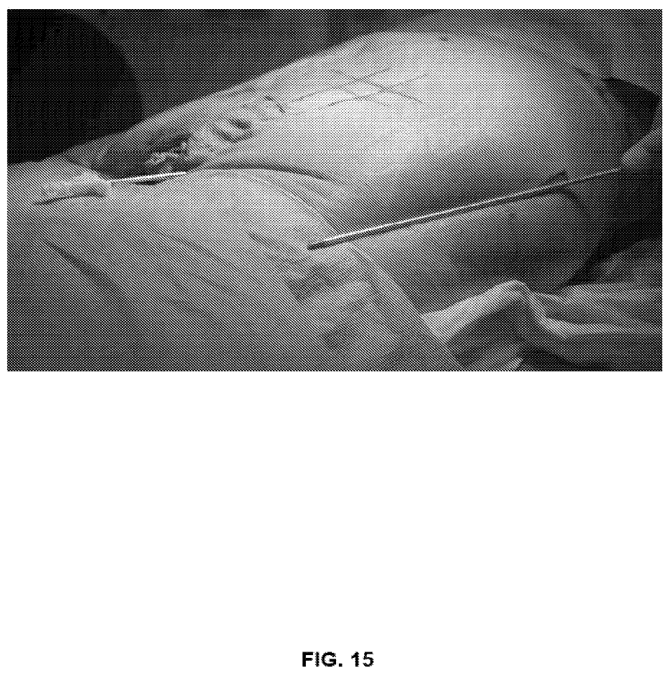

In some embodiments, skin medial to ischial tuberosity may be cut at the level determined from lateral measurements. In some embodiments, the skin incision is about 1 cm. In some embodiments, the skin incision is more than 1 cm. In some embodiments, the soft tissue at and near the incision is opened. In some embodiments, the soft tissue at and near the incision may be opened to allow for a clear palpation of ischial tuberosity. In some embodiments, the soft tissue at and near the incision may be opened to allow for flexibility on angulation of introducer. In some embodiments, introducer is advanced toward marked as an area of interest through the incision opening and to a depth as marked on the individual laterally. In some embodiments, introducer is advanced with the introducer needle retracted. In some embodiments, the advancement of the introducer through the skin opening may be followed by imaging in AP view as shown in FIG. 15. In some embodiments, the imaging comprises fluoroscopy. In some embodiments, the imaging comprises X-ray. In some embodiments, the incision and introducer are at the same horizontal level as shown in FIG. 15. FIG. 15 shows an exemplary embodiment of the lateral depth marking from X-ray.

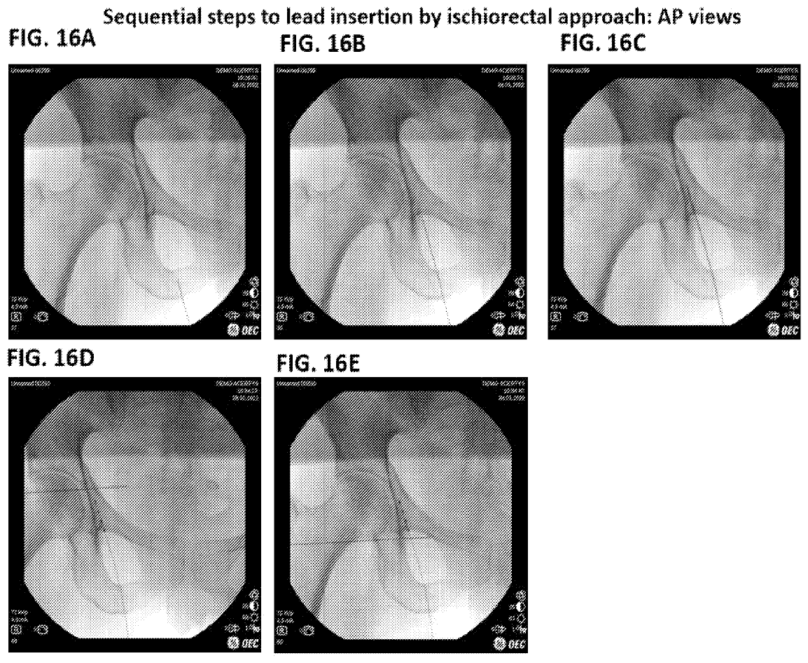

In some embodiments, the internal needle is deployed using a screw lock mechanism when the introducer meets resistance after advancing within the soft tissue through the skin opening. In some embodiments, the introducer position is advanced a distance sufficient to perforate or pass close to the sacrotuberous ligament with the introducer needle to access the pudendal nerve. In some embodiments, the distance sufficient to perforate or pass close to the sacrotuberous ligament is at least about 1, 2, 3, 4, 5, 6, 7, 8, 9, or 10 cm. In some embodiments, the distance sufficient to perforate or pass close to the sacrotuberous ligament is about 4 cm. In some embodiments, the distance sufficient to perforate or pass close to the sacrotuberous ligament is at least 1 cm. In some embodiments, the introducer needle is in an unretracted position while the introducer is advanced. In some embodiments, the introducer needle is in a retracted position while the introducer is advanced to the sacrotuberous ligament, and the needle protrudes to an unretracted position to perforate the sacrotuberous ligament. In some embodiments, the introducer needle is retracted after the sacrotuberous ligament is perforated. In some embodiments, the obturator is left in place on the pudendal nerve after the introducer needle is retracted. In some embodiments, a distal contact point of the lead is connected to an electrical connector. In some embodiments, the distal contact point of the lead is connected to a J-hook electrical connector. In some embodiments, the introducer is advanced from bottom to top of the area of interest guided by X-ray and EMG measurement as shown in FIGS. 16A-E. In some embodiments, the introducer is advanced at least about 0.5, 1, 2, 3, 4, or 5 cm. In some embodiments, the introducer is advanced about 2.5 cm. In some embodiments, the introducer is advanced no more than about 2, 3, 4, or 5 cm. FIGS. 16A-E show exemplary embodiments of sequential fluoroscopic images of insertion of the introducer and lead by ischiorectal approach toward the ischial spine in A-P view. The electrodes on the lead may be seen as four dots along distal end of the lead targeting the pudendal nerve in FIGS. 16D and 16E.

In some embodiments, one or more electrodes on the lead are stimulated as electrical signal and/or contraction of muscles innervated by the pudendal nerve is observed. In some embodiments, the placement of the lead on the pudendal nerve may be adjusted based on the electrical signal, contraction of muscles, or a combination thereof. In some embodiments, one or more electrodes on the lead are stimulated as muscle contraction of anal sphincter is observed. In some embodiments, all electrodes on the lead are stimulated as muscle contraction of anal sphincter is observed. In some embodiments, one or more electrodes on the lead are stimulated as muscle contraction of muscles of the pelvic floor is observed. In some embodiments, all electrodes on the lead are stimulated as muscle contraction of urinary sphincter is observed. In some embodiments, one or more electrodes on the lead are stimulated as muscle contraction of urinary sphincter is observed. In some embodiments, the muscle contraction and EMG measurements of the pelvic floor are observed while the electrodes are stimulated. In some embodiments, the muscle contraction is observed while the electrodes are stimulated. In some embodiments, the EMG measurements of the pelvic floor is taken while the electrodes are stimulated. In some embodiments, the transvaginal EMG measurements are taken while one or more electrodes are stimulated. In some embodiments, the transvaginal EMG measurements are taken while all electrodes are stimulated. In some embodiments, the EMG measurements are taken using an intravaginal device. In some embodiments, the intravaginal device is a vaginal EMG probe.

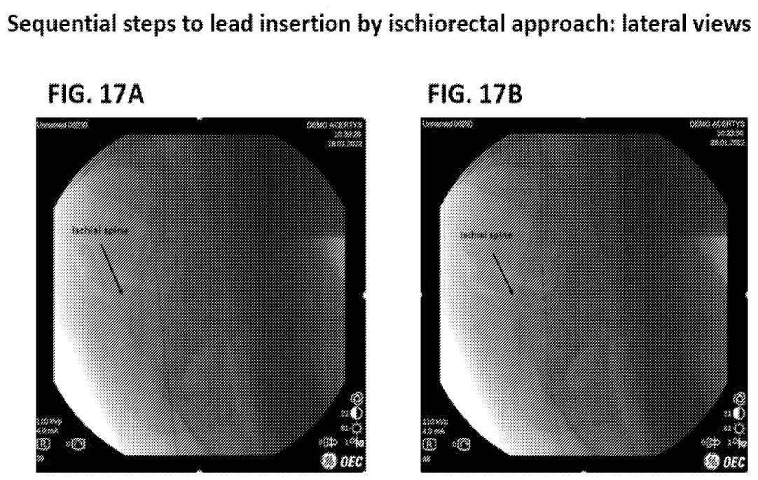

In some embodiments, the position of the lead on the pudendal nerve is adjusted based on the muscle contraction, the EMG measurements, or a combination thereof to achieve a desired muscle response to the electrical stimulation. In some embodiments, the desired muscle response comprises contraction of the muscle at a specified electrical stimulation parameter, including but not limited to amplitude, frequency, and duration. In some embodiments, the desired muscle response comprises a lack of muscle contraction at a specified electrical stimulation parameter. In some embodiments, EMG or evidence of muscle contraction is recorded at a low stimulation amplitude at one or more contact points. In some embodiments, EMG or evidence of muscle contraction is recorded at a low stimulation amplitude at two contact points. In some embodiments, FMG or evidence of muscle contraction is recorded at a low stimulation amplitude at three contact points. In some embodiments, the obturator may be removed, and the lead may be introduced up to the marking point. In some embodiments, the lead electrodes may be left at the same position as the introducer ring electrodes. In some embodiments, the introducer may be removed completely while keeping the lead stable at the desired position using continuous radiological imaging, including but not limited to fluoroscopy. In some embodiments, adequate position of the lead may be verified by electrical stimulation or radiological imaging, including but not limited to X-ray, or a combination thereof. FIGS. 17A-B show exemplary embodiments of sequential fluoroscopic images of insertion of the introducer and the lead after the introducer is removed by ischiorectal approach in lateral view, where the electrodes on the lead may be seen as four dots along the pudendal nerve in FIG. 17B.

### Low Gluteal Approach

Provided herein are methods, devices, and systems to allow for accessing the pudendal nerve by low gluteal approach with little damage to tissues surrounding the pudendal nerve. In some embodiments, the introducer may access the pudendal nerve by a low gluteal approach, where the introducer is directed to pass in the space between sacrotuberous ligament and sacrospinous ligament and to pass anteriorly in the ischiorectal fossa below the pelvic floor to place the lead on the anterior branches of the pudendal nerve to stimulate the pudendal nerve and the dorsal genital nerve.

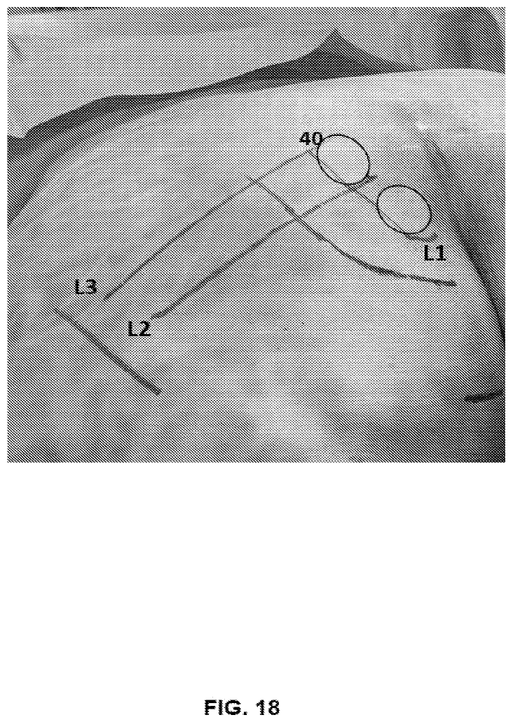

In some embodiments, an electrode needle is used to determine presence of an electrophysiological response before proceeding with introducer insertion. In some embodiments, an electrode needle is used to determine laterality of a desired electrophysiological response before proceeding with introducer insertion. In some cases, the desired electrophysiological response may comprise a detectable EMG response at a stimulation amplitude at less than about 4 milliampere. In some embodiments, skin that is lateral from the inner brim of the ischial bone as determined by a prior AP measurement may be cut. In some embodiments, the incision site based on the prior AP measurement is at a location equidistant between L2 line and L1 line on anterior border of ischial bone. In some embodiments, the skin incision is about 1 cm to about 2 cm, or about 1 cm to about 3 cm, about 1 cm to about 4 cm, or about 1 cm to about 5 cm. In some embodiments, the soft tissue at and near the incision may be opened to allow for a clear palpation of the ischial bone. In some embodiments, the soft tissue at and near the incision may be opened to allow for flexibility on angulation of introducer. FIG. 18 shows an exemplary embodiment of the insertion point for the low gluteal approach as indicated by the distal oval, which are also shown in FIG. 13B.

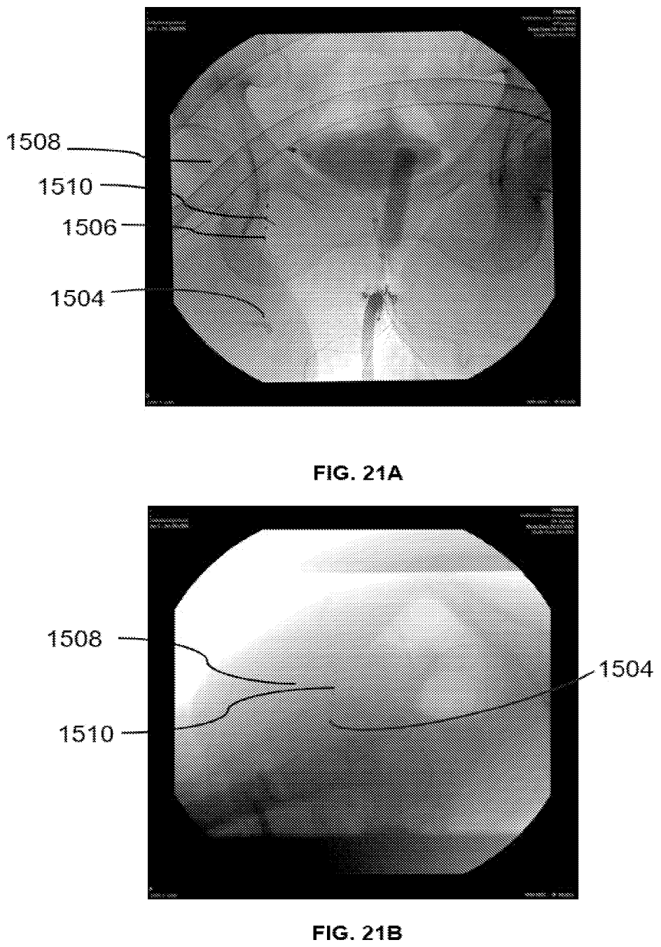

In some embodiments, the introducer is advanced and probed to find the location where the pudendal nerve angulates into Alcock's canal. In some embodiments, the location where the pudendal nerve angulates into Alcock's canal may be inferior to the L2 line. In some embodiments, the position of a first electrode lead (e.g., a trunk electrode lead 1504), placed by the ischiorectal approach, can be used as a guide for determining the site of angulation of the pudendal nerve as shown in FIGS. 21A-21B and subsequent placement of a second electrode 1508 (e.g., an anterior electrode). Responses from the different contact points 1506 (e.g., one or more electrodes disposed on the electrode lead, described elsewhere herein) on first electrode lead 1504 may indicate where the pudendal nerve turns forward in the region of the ischial spine and may mark the starting point for the second electrode lead 1508. In some embodiments, oblique X-ray views may be used to help determine the trajectory of the pudendal nerve as angulated forward to guide the placement of the second electrode lead 1508, as shown in FIG. 21B. The region of where the pudendal nerve turns forward can be visualized as the region where the first electrode lead and second electrode lead cross 1510, as shown in FIGS. 21A-21B. In some embodiments, the introducer with needle is advanced downward onto the ischial bone to help with orientation. In some embodiments, the introducer is angled anteriorly to skirt medial border of the ischial bone. In some embodiments, the needle is retracted, and the introducer is advanced slightly into gap between the sacrotuberous ligament and the sacrospinous ligament. In some embodiments, the introducer with the needle in a retracted position is advanced slightly downwards until resistance is felt. In some embodiments, the resistance to advancing the introducer occurs when the introducer is in contact with a bone. In some embodiments, the introducer is angulated medially and inserted a distance to reach the pudendal nerve. In some embodiments, the distance to reach the pudendal nerve is at least about 1 mm, 2 mm, 3 mm, 4 mm, 5 mm, 6 mm, 7 mm, 8 mm, 9 mm, or 10 mm. In some embodiments, the distance to reach the pudendal nerve is about 5 mm.

In some embodiments, a distal contact point of the lead is connected to an electrical connector. In some embodiments, the electrical connector is a J-hook electrical connector. In some embodiments, the EMG responses are evaluated after the lead is connected to the electrical connector. In some embodiments, a J-hook electrical connector is connected to most distal contact point of the lead and the EMG responses are evaluated. In some embodiments, the electrical connector provides electrical stimulation through the electrodes on the lead to the tissue in contact with the electrode. In some embodiments, the electrical connector provides electrical stimulation to the pudendal nerve through the electrodes on the lead that are in contact with the pudendal nerve.

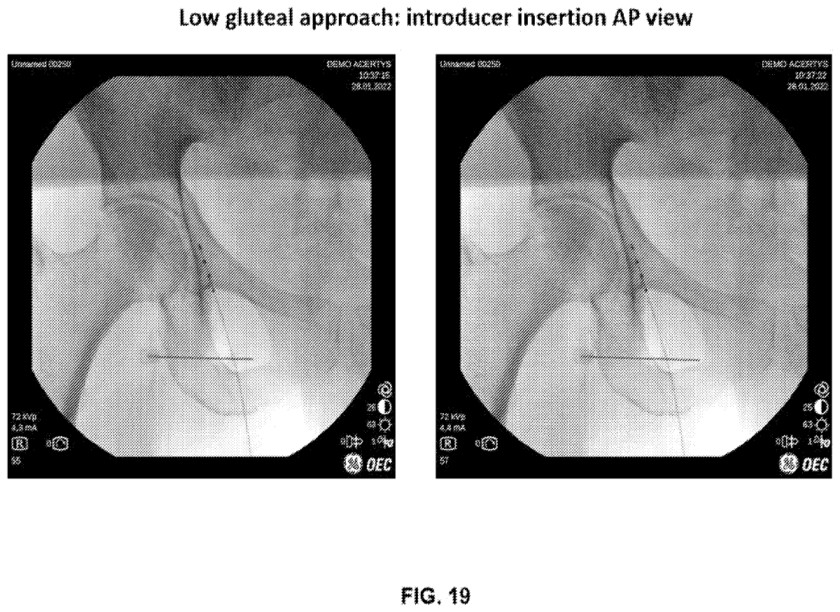

In some embodiments, the introducer is advanced further into the tissue through the opening with the needle in the retracted position and the lead connected to the electrical connector. In some embodiments, the introducer with the needle in the retracted position and the lead connected to the electrical connector is advanced further into the tissue in the forward and medial direction. In some embodiments, the introducer is advanced to follow the curve formed by the inferior pubic ramus at the inferior border of the obturator foramen. In some embodiments, the advancement of the introducer is guided by radiological imaging, including but not limited to fluoroscopy. In some embodiments, the advancement of the introducer is followed fluoroscopically in the AP view. In some embodiments, the advancement of the introducer is guided by EMG measurements to determine the location along the pudendal nerve to place the lead that results in desired muscle response to electrical stimulation. In some embodiments, the EMG measurement is taken of one or more of anal sphincter, urethral sphincter, muscles of the pelvic floor, muscles involved with urinary incontinence, or muscles involved in fecal incontinence. In some embodiments, the advancement of the introducer is guided by radiological imaging, EMG measurements, or a combination thereof. In some embodiments, the advancement of the introducer is guided by radiological imaging, EMG measurements, or a combination thereof, so that the introducer is observed as it is advanced to follow the curve formed by the inferior pubic ramus at the inferior border of the obturator foramen. In some embodiments, the introducer may eventually point in the direction of the pubic symphysis as shown in FIG. 19. FIG. 19 shows an exemplary embodiment of fluoroscopic images of the insertion of the introducer by the low gluteal approach in the AP view, where the introducer is directed along the line of the inferior border of the obturator foramen towards the pubic symphysis. The electrodes on the lead are shown as four round dots in the fluoroscopic images.

In some embodiments, one or more electrodes on the lead are stimulated as electrical signal and/or contraction of muscles innervated by the pudendal nerve is observed. In some embodiments, the placement of the lead on the pudendal nerve may be adjusted based on the electrical signal, contraction of muscles, or a combination thereof. In some embodiments, the lead position may be adjusted until a satisfactory FMG response is measured. In some embodiments, one or more electrodes on the lead are stimulated as muscle contraction of anal sphincter is observed. In some embodiments, all electrodes on the lead are stimulated as muscle contraction of anal sphincter is observed. In some embodiments, one or more electrodes on the lead are stimulated as muscle contraction of muscles of the pelvic floor is observed. In some embodiments, all electrodes on the lead are stimulated as muscle contraction of urinary sphincter is observed. In some embodiments, one or more electrodes on the lead are stimulated as muscle contraction of urinary sphincter is observed. In some embodiments, the muscle contraction of urinary sphincter is measured directly using a transducer on a urinary catheter. In some embodiments, the muscle contraction and EMG measurements of the pelvic floor are observed while the electrodes are stimulated. In some embodiments, the muscle contraction is observed while the electrodes are stimulated. In some embodiments, the EMG measurements of the pelvic floor at low stimulation amplitude are taken while one or more electrodes are stimulated. In some embodiments, the EMG measurements of the pelvic floor at low stimulation amplitude are taken while all electrodes are stimulated. In some embodiments, all electrodes on the introducer are stimulated to record the EMG of the pelvic floor muscles at low stimulation amplitude at three contact points. In some embodiments, the EMG measurements are taken using an intravaginal Maple device. In some embodiments, the EMG data is recorded in the operative record during the implantation procedure. In some embodiments, the obturator is removed, and the lead is introduced up to the marking point so that the lead electrodes are left at the same position as the introducer electrodes.

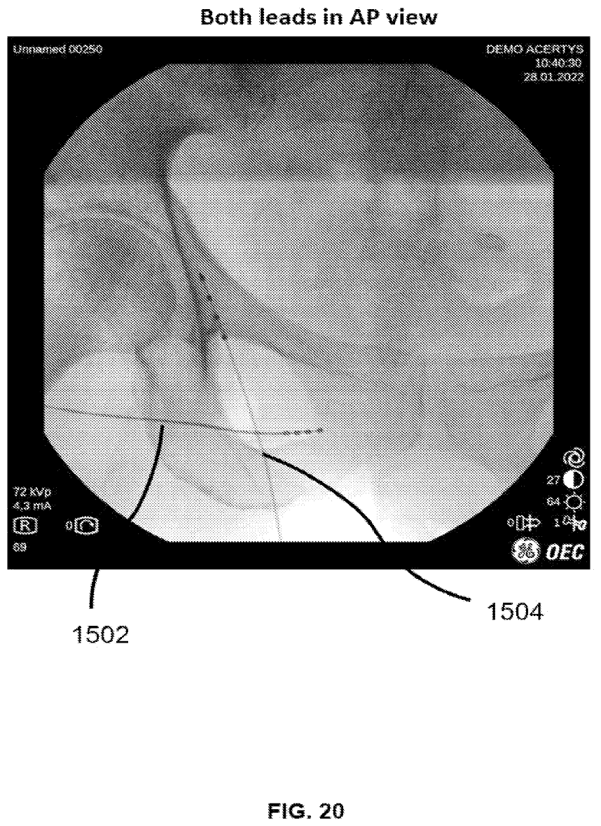

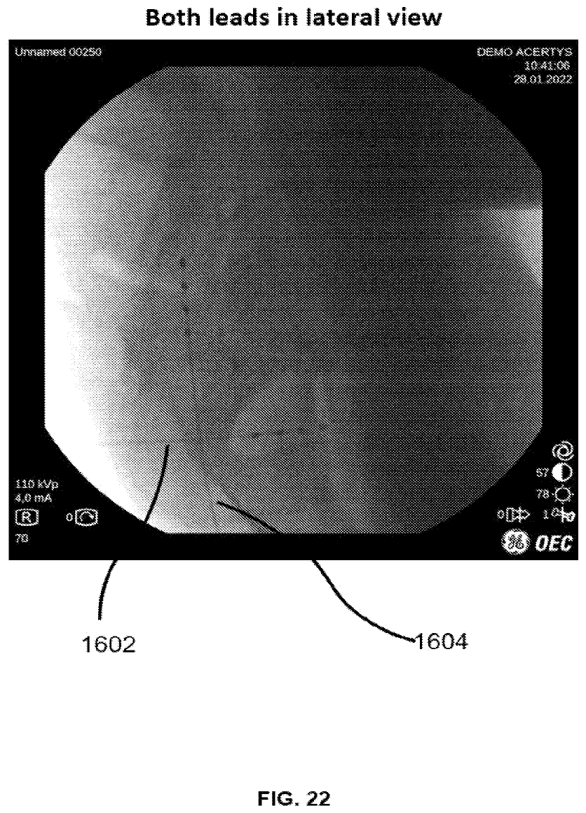

In some embodiments, the position of the lead on the pudendal nerve is adjusted based on the muscle contraction, the EMG measurements, or a combination thereof to achieve a desired muscle response to the electrical stimulation. In some embodiments, the desired muscle response comprises contraction of the muscle at a specified electrical stimulation parameter, including but not limited to amplitude, frequency, and duration. In some embodiments, the desired muscle response comprises a lack of muscle contraction at a specified electrical stimulation parameter. In some embodiments, EMG or evidence of muscle contraction is recorded at a low stimulation amplitude at one or more contact points. In some embodiments, EMG or evidence of muscle contraction is recorded at a low stimulation amplitude at two contact points. In some embodiments, EMG or evidence of muscle contraction is recorded at a low stimulation amplitude at three contact points. In some embodiments, the obturator may be removed, and the lead may be introduced up to the marking point. In some embodiments, the lead electrodes may be left at the same position as the introducer ring electrodes. In some embodiments, the introducer may be removed completely while keeping the lead stable at the desired position using continuous radiological imaging, including but not limited to fluoroscopy. In some embodiments, adequate position of the lead may be verified by electrical stimulation or radiological imaging, including but not limited to X-ray, or a combination thereof. FIG. 20 shows an exemplary embodiment of a fluoroscopic image in the AP view of insertion of the lead by the low gluteal approach after the introducer has been removed and two leads with four electrodes on each lead (as indicated by four circles) 1502, 1504 are in place on the pudendal nerve. FIG. 22 shows an exemplary embodiment of a fluoroscopic image in the lateral view of insertion of the lead by the low gluteal approach and confirmation of position of two leads 1602, 6504.

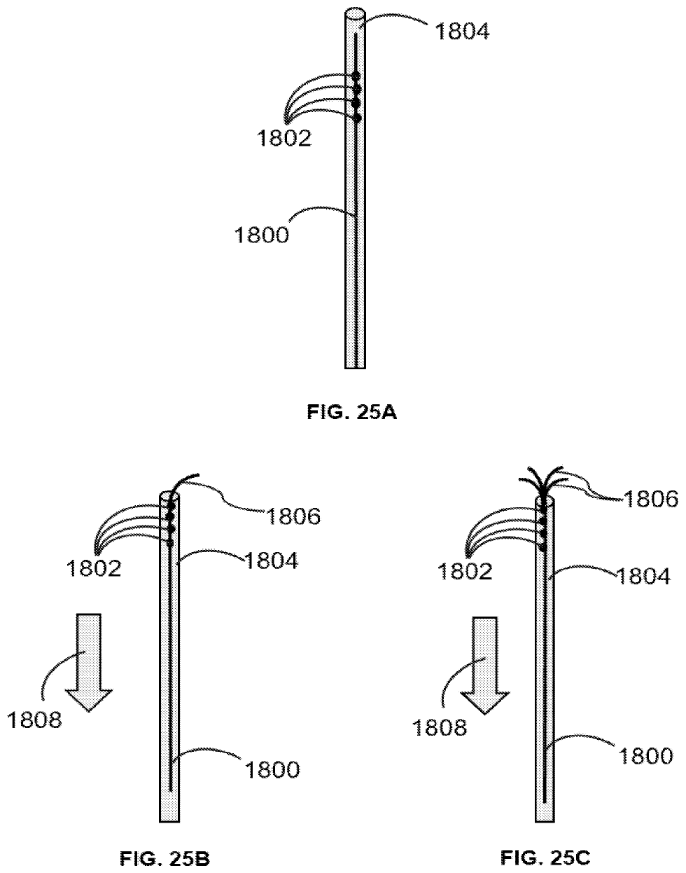

In some embodiments, the retraction 1808 of the introducer 1804 from the insertion site may allow for a change in the conformation of the electrode lead tip 1806. In some embodiments, the change in the conformation of the electrode lead tip 1806 may aid in preventing movement of the electrode lead 1800 from the placement site. In some embodiments, the change in conformation of the electrode lead tip 1806 may comprise an angulation of the electrode tip 1806, as shown in FIGS. 25B-25C. In some embodiments, the change in conformation of the electrode lead tip 1806 may comprise a change in the angle or orientation of the electrode lead tip. In some embodiments, the electrode lead tip 1806 may change in conformation from straight to curved with the retraction 1808 of the introducer 1804. In some embodiments, the introducer 1804 may help to keep the electrode lead tip 1806 in a linear conformation prior to retraction 1808 of the introducer relative to the electrode lead tip as shown in FIG. 25A. In some embodiments, the curvature of the electrode lead tip 1806 may be reduced when the introducer 1804 is placed over the electrode lead tip 1806. In some embodiments, the electrode lead tip 1806 may be straightened when the introducer 1804 is placed over the electrode lead tip 1806. In some embodiments, this curvature may be conferred by memory in the electrode lead 1800 design such that on protrusion from the lead introducer 1804 the curvature forms spontaneously. In some cases, the electrode lead 1800 may comprise a shape memory material e.g., nitinol, that on protrusion from the lead introducer 1804 forms into the shape set of the shape memory material. In some embodiments, the curvature may be re-straightened when retracted back inside the lead introducer 1804. In some embodiments, the lead curvature may prevent movement of the electrode lead tip 1806 once the electrode lead 1800 and corresponding one or more electrodes 1802 on the lead 1800 are positioned in a subject.

In some embodiments, retraction 1808 of the introducer 1804 may allow for the electrode lead tip 1806 to curve. In some embodiments, the electrode lead 1800 may have a curved tip 1806 at a distal end of the electrode lead 1800. In some embodiments, the electrode lead may have a flexible tip with a low mechanical stiffness. In some embodiments, the curvature of the lead tip may comprise up to about 20 degrees, up to about 30 degrees, up to about 40 degrees, up to about 50 degrees, up to about 60 degrees, up to about 90 degrees, up to about 100 degrees, up to about 120 degrees, up to about 140 degrees, up to about 160 degrees, or up to about 180 degrees curvature. In some embodiments, the curvature of the lead tip may be at most about 180 degrees, at most about 160 degrees, at most about 140 degrees, at most about 120 degrees, at most about 100 degrees, at most about 90 degrees, at most about 60 degrees, at most about 50 degrees, at most about 40 degrees, at most about 30 degrees, or at most about 20 degrees. In some embodiments, retraction 1808 of the introducer 1804 may expose one or more of a plurality of lead fixation methods that are customized to lead design, e.g., as seen in FIG. 25C. In some embodiments, the electrode lead tip 1806 may comprise a plurality of wires. In some embodiments, one or more of the wires in the plurality of wires may be curved. In some embodiments, one or more of the wires in the plurality of wires may form a flower-like arrangement, as shown in FIG. 25C, due to their flexibility. In some embodiments, when the lead wires are uncovered, the flower-like arrangement may form spontaneously due to the material properties of the lead wire. In some embodiments, the lead tip 1806 may comprise a flexible material with low mechanical stiffness. In some embodiments, the flower-like arrangement formed by the plurality of the electrode lead tip 1806 may change into a substantially straightened conformation when the plurality of the electrode lead tip 1806 is retracted inside the lead introducer 1804. In some embodiments, the flower-like arrangement may prevent movement of the lead tip 1806 once the electrode lead 1800 is positioned. In some embodiments, the flower-like arrangement may allow for the electrode lead tip 1806 to hook into a surrounding tissue and secures the electrode lead 1800 in the target location.

In some embodiments, when the electrode lead tip 1806 is covered by the lead introducer 1804, the electrode lead tip 1806 may have a different conformation than when the lead introducer 1804 is retracted 1808 away from the electrode lead tip 1806. In some embodiments, the electrode lead tip 1806 may comprise a first conformation when the tip is contained within the lead introducer 1804 and a second conformation when the tip 1806 is exposed from the lead introducer. In some embodiments, the first conformation may be substantially straight. In some embodiments, the second conformation may be curved. In some embodiments, the first conformation and the second conformation may be different.

### Lead Fixation

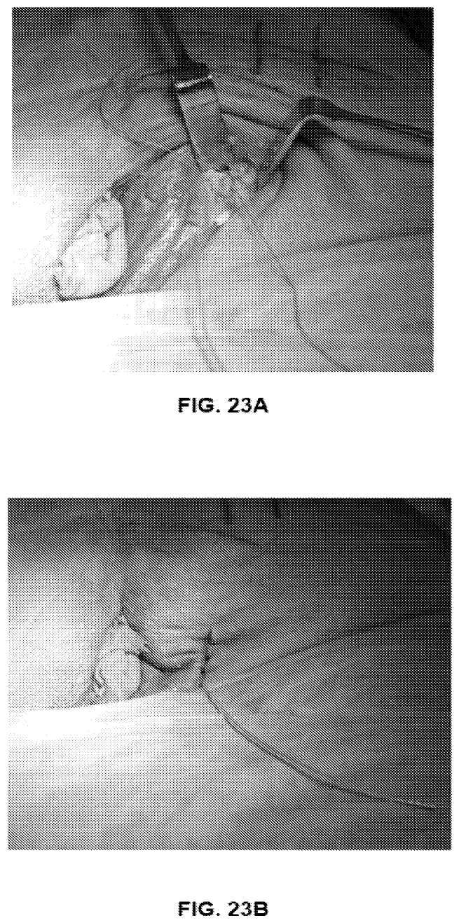

In some embodiments, one or more leads may be fixed in place on the pudendal nerve by various fixation methods. In some cases, one or more leads may be fixed to the tissues surrounding the pudendal nerve. In some cases, the electrode lead may be fixed with a passive anchor. In some cases, the one or more leads may comprise one or more anchoring features (e.g., one or more tines and/or fins disposed on a surface of the lead). The one or more anchoring features disposed on a surface of lead may fix and/or secure the lead in tissue when deployed. In some cases, the electrode lead may be supplied with the passive anchor for lead fixation. In some cases, the anchor may comprise an internal anchor layer disposed in contact with a surface of the lead and an outer layer disposed in contact with human tissue. In some cases, the anchor outer layer may have one or more grooves. In some cases, the anchor outer layer may comprise at least two grooves. In some cases, each groove may be disposed on the outer anchor layer such that compression is transferred to the internal anchor layer. In some cases, the internal anchor layer may be made of titanium. In some cases, the anchor may be compressed over the lead by tight ligatures in the grooves. In some instances, the anchor may be compressed onto a surface of the lead by a small-mounted screw assembly which can be tightened using a miniaturized torque wrench to a pre-specified pressure limit. In some cases, one anchor may be used per electrode lead. In some cases, additional anchors may be used on each lead. In some cases, the lead may be in a target position to treat incontinence as determined by radiology, electrophysiology, or a combination thereof. In some cases, after the lead is in a target position to treat incontinence, the anchor may be positioned onto the lead e.g., by sliding the anchor onto and/or over the lead, without displacing the lead position. In some cases, an anchor may be slid onto the lead and a suture taken through periosteum of ischium using a needle. FIG. 23A show an exemplary embodiment of an anchor that has been slid onto the lead and a polyester stay suture taken through periosteum of ischium using a J needle. In some cases, the suture may be tied around the groove of the passive anchor to compress the suture onto the lead. As shown in FIG. 23B, in some cases, the anchor may be drawn into the tissue by tightening the stay suture after a second ligature is used on the second groove of the anchor.

In some cases, the anchor may be deployed into the tissue. In some cases, the anchor may be deployed deep in the tissue. In some cases, the depth of deployment is between 1 cm and 5 cm. In some cases, dilation of the soft tissues deep to the site of skin incision may allow for advancement of the electrode lead and anchor. In some cases, dilatation may be achieved using a dissection tool or an over-sheath. In some cases, a tissue tunnel may be created using a standard surgical instrumentation such as a scalpel. In some cases, dilatation of the tissue tunnel may use a sheath to bluntly create the tissue tunnel of a diameter sufficient to permit smooth subsequent advancement of the anchor and the electrode lead. In some cases, the sheath may comprise a sufficiently high mechanical stiffness to create the tissue tunnel from the incision site. In some cases, the dilated tissue tunnel may be created in tissue adjacent to the ischial bone and fascial/ligamentous insertions.

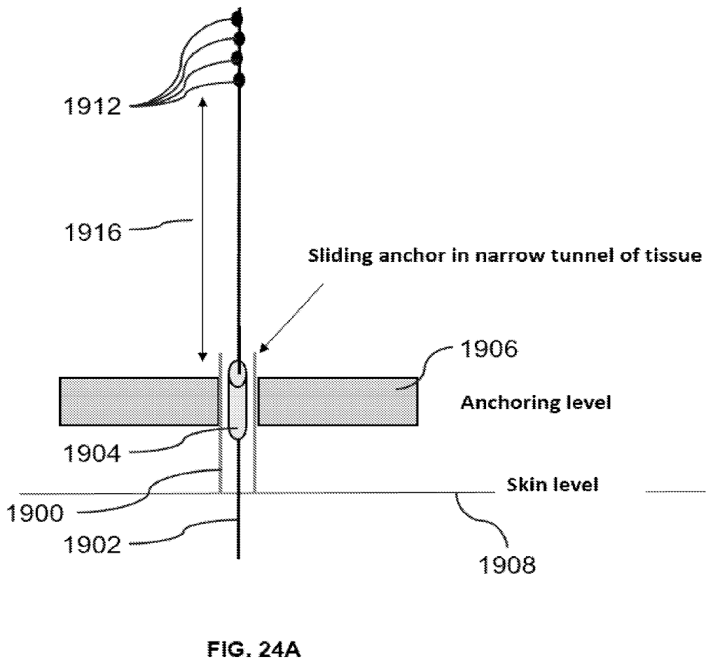

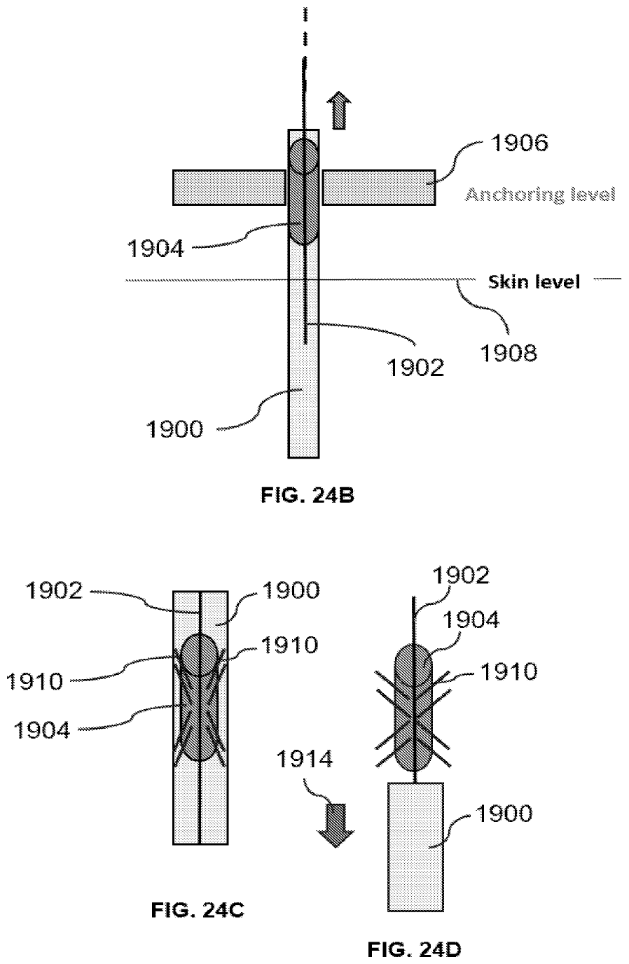

In some cases, the anchor 1904 may be threaded onto and advanced along the length the lead 1902 and secured at a target site on the lead, as shown in FIG. 24A. In some cases, an anchoring device may comprise an anchor 1904, an electrode lead 1902, and an over-sheath 1900 having a lumen configured to cover the anchor 1904 and the lead 1902, as seen in FIGS. 24A-24C. In some cases, the anchor 1904 comprises one or more securing arms 1910, as shown in FIG. 24C and 24D. In some cases, the securing arm 1910 may be located on an outward surface of the anchor. In some cases, the securing arm 1910 may comprise one or more a tines and/or one or more fins. In some cases, the securing arm 1910 may comprise one or more spring-loaded tines. In some cases, the securing arm 1910 may prevent the anchor 1904 from exiting the lumen of the over-sheath 1900 while and/or during positioning of the anchor 1904 to an anchoring position prior to be being pushed out of over-sheath 1900. In some cases, the spring-loaded securing arm can releasably secure the anchor 1904 within the lumen of the over-sheath 1900, where the anchor 1904 can be released from the over-sheath 1900 covering the anchor 1904. In some cases, the anchor 1904 may hold the electrode lead 1902 while covered by the over-sheath 1900 to prevent unwanted deployment of the electrode lead 1902 prior to deployment of the anchor 1904 and electrode lead 1902 by e.g., pushing the anchor 1904 and/or electrode lead out of the over-sheath 1900. In some cases, the over-sheath 1900 may be releasable from the anchor 1904 and the lead 1902.

In some cases, one or more anchors 1904 may be advanced onto the lead 1902 and into the soft tissue space 1906 created by dilatation (e.g., tunnel of tissue) before being secured to the tissue 1908 as shown in FIG. 24A. In some cases, the anchor 1904 may be positioned at a distance of at least about 1 cm, at least about 2 cm, at least about 3 cm, at least about 4 cm, or at least about 5 cm from a skin surface 1908. In some cases, the over-sheath 1900 that covers the anchor 1904, electrodes 1912, and/or lead 1902 may be advanced into the target location 1906 within the soft tissue space created by dilatation. In some cases, the anchor 1904 may comprise a distance 1916 from the electrodes 1912. In some cases, the distance 1916 may comprise a distance of at least about 5 cm, at least about 10 cm, at least about 15 cm, at least about 20 cm, at least about 25 cm, at least about 30 cm, at least about 35 cm, at least about 40 cm, at least about 45 cm, at least about 50 cm, at least about 55 cm, or at least about 60 cm. In some cases, the distance 1916 may comprise a distance of at most about 5 cm, at most about 10 cm, at most about 15 cm, at most about 20 cm, at most about 25 cm, at most about 30 cm, at most about 35 cm, at most about 40 cm, at most about 45 cm, at most about 50 cm, at most about 55 cm, or at most about 50 cm. In some cases, the over-sheath 1900 may be retracted 1914 from the anchor 1904 and the lead 1902. as shown in FIG. 24D. In some cases, the anchor 1904 may be secured to the lead 1902. In some cases, the securing arm 1910 on the anchor 1904 may expand outward when the over-sheath 1900 is retracted 1914, as shown in FIG. 24D. In some cases, the outward expansion of the securing arm 1910 may secure the anchor 1904 to the tissue. In some cases, the outward expansion of the securing arm 1910 may secure the anchor 1904 and the lead 1902, that the anchor 1904 is holding to the tissue. In some cases, the anchor 1904 may grip the lead 1902 and the surrounding tissue and prevent movement of the lead 1902. In some cases, the securing arm 1910 of the anchor 1904 may grip the lead 1902 and the surrounding tissue and prevents movement of the lead 1902.

In some cases, one or more passive anchors may be used. In some cases, the anchor(s) can be advanced onto the electrode lead by manual advancement (e.g., pushing by hand or basic surgical instrument such as a clip). In some cases, the over-sheath 1900 may be used to advance the anchor 1904 into position on the electrode lead 1902 as shown in FIG. 24B. In some cases, the anchor 1904 may be contained within the over-sheath 1900 before deployment as shown in FIG. 24C. In some instances, the anchor 1904 may expand radially outward from a central axis of the anchor 1904 upon deployment out of the over sheath 1900. In some cases, the radially outward expanding anchor 1904 may be coupled to the lead 1902 and thus anchor the lead 1902 and electrode at a target tissue 1906.

In some cases, one or more fixation methods may be used to secure the anchor to the lead. In some cases, one or more fixation methods may be used to secure the anchor to the surrounding soft tissues. In some cases, the anchor may be fixed to ligamentous, fascial and/or periosteal tissues. In some cases, standard surgical approaches for fixation to tissues may be used, e.g., suturing. In some cases, one or more physical features of the anchor may be used for fixation to the tissues. In some cases, the anchor may be secured to the electrode lead by the frictional force between the anchor and the lead. In some cases, ligatures may be used to compress the anchor onto the electrode lead. In some cases, suture materials may be used to provide ligatures for compression of the anchor onto the lead. In some cases, a small-mounted screw assembly may be used, which can be tightened using a miniaturized torque wrench to a pre-specified pressure limit. In some cases, a torque wrench designed to allow tightening of the mounted screw deep in soft tissues may be used to secure the screw assembly. In some cases, surgical ligating clips may be used to compress the anchor onto the electrode lead to secure the lead. In some cases, surgical ligating clips may be applied using a ligating clip applier. In some cases, the anchor may be used to secure the anchor to electrode lead and surrounding soft tissues. In some cases, the anchor 1904 may deploy automatically on extrusion from the over-sheath 1900 or retraction of the over-sheath 1900 as shown in FIG. 24C. In some cases, retraction of the over-sheath 1900 may activate the anchor 1904. In some cases, the anchor 1904 may be pushed out from the over-sheath 1900 to enable activation. In some cases, the anchor may be pushed out from the over-sheath using a pushing member e.g., a pushing rod or tube. In some cases, a mechanism to secure the anchor 1904 to the tissue may be activated automatically on extrusion of the anchor 1904 from the over-sheath 1900. In some cases, the mechanism may include the activation of spring-loaded tines 1910 as shown in FIG. 24C. In some cases, the activating mechanism may incorporate a clipping function to the lead 1902 and to surrounding tissues.

In some cases, the anchor may be advanced into the wound using suitable retraction of the wound edges. In some cases, the anchor may be advanced such that the anchor is adjacent to but not touching the deep fascial overlying the ischial bone or ligaments. In some cases, the distal groove lying deepest to the skin may be used to anchor the lead to the periosteum of the ischium or adjacent ligamentous tissue. In some cases, a suture on a suture needle may be used to suture the lead onto the groove of the anchor. In some cases, the suture may comprise a non-absorbable braided suture material e.g., polyester suture. In some cases, the suture needle comprises may comprise a J needle. In some cases, the suture needle may be advanced through the periosteum/ligamentous tissue and the suture is wound around the groove. In some cases, the suture may be tightened firmly to compress the enclosed clip over the lead and to fix the lead against retraction. In some cases, the proximal groove lying more superficially may be used to further compress the anchor on the lead using a suture. In some cases, the suture may comprise a polyester suture. In some cases, the anchor may be tested against light retraction on the lead without stretching the lead to ensure secure lead fixation.

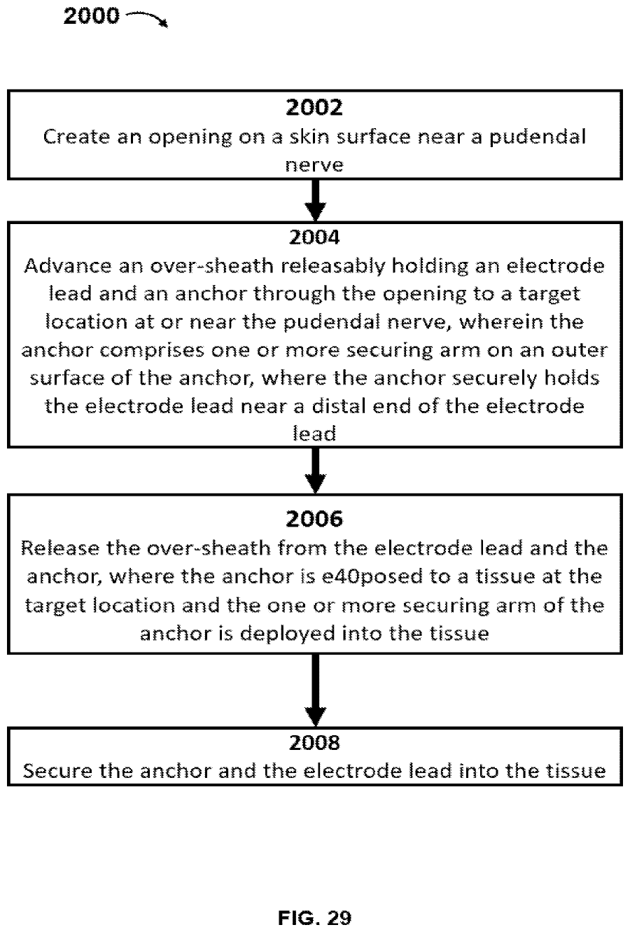

In some cases, the methods, devices, and systems described herein may comprise a method of securing an electrode lead to a tissue near a pudendal nerve 2000, as seen in FIG. 29. In some instances, the method may comprise: (a) creating an opening on a skin surface near a pudendal nerve 2002; (b) advancing an over-sheath releasably holding an electrode lead and an anchor through the opening to a target location at or near the pudendal nerve, where the anchor may comprises one or more securing arm on an outer surface of the anchor, and where the anchor may securely hold the electrode lead near a distal end of the electrode lead 2004; (c) releasing the over-sheath from the electrode lead and the anchor, where the anchor may be exposed to a tissue at the target location and the one or more securing arm of the anchor may be deployed into the tissue 2006; and (d) securing the anchor and/or the electrode lead into the tissue. In some cases, the method of securing an electrode lead to a tissue near a pudendal nerve may further comprise removing the over-sheath from the opening 2008. In some instances, creating the opening may comprise dilatating a tissue around the opening. The dilatation may be achieved by a dissection tool. The dilatation may be achieved by the over-sheath. In some instances, the tissue may be adjacent to ischial bone and fascial or ligamentous insertions. In some cases, the deployment of the one or more securing arm of the anchor into the tissue may comprises the one or more securing arms expanding outwardly from the anchor. In some instances, the one or more securing arms may be spring-loaded. In some cases, the anchor may comprise a passive anchor. In some cases, the tissue to which the anchor and the electrode lead are secured may comprise one or more of ligamentous, fascial, or periosteal tissues. In some instances, the securing may further comprise suturing of the anchor to the tissue. In some cases, the anchor may securely hold the electrode lead by a frictional force between the anchor and the electrode lead. In some cases, the anchor may securely hold the electrode lead by ligature of the anchor to the electrode lead. In some instances, the ligature may comprise compression of the anchor onto the electrode lead. In some cases, the ligature comprises using a screw. In some instances, the ligature may comprise using a ligating clip. In some instances, the electrode lead may comprise one or more distal lead tips. In some instances, the one or more distal lead tips may be configured to be substantially straight when covered by the introducer and/or the over-sheath and substantially curved when the introducer and/or over-sheath is retracted from the distal tip. In some cases, the curve of the one or more distal lead tips may aid in securing the electrode lead to the tissue.

### IPG Pocket Formation

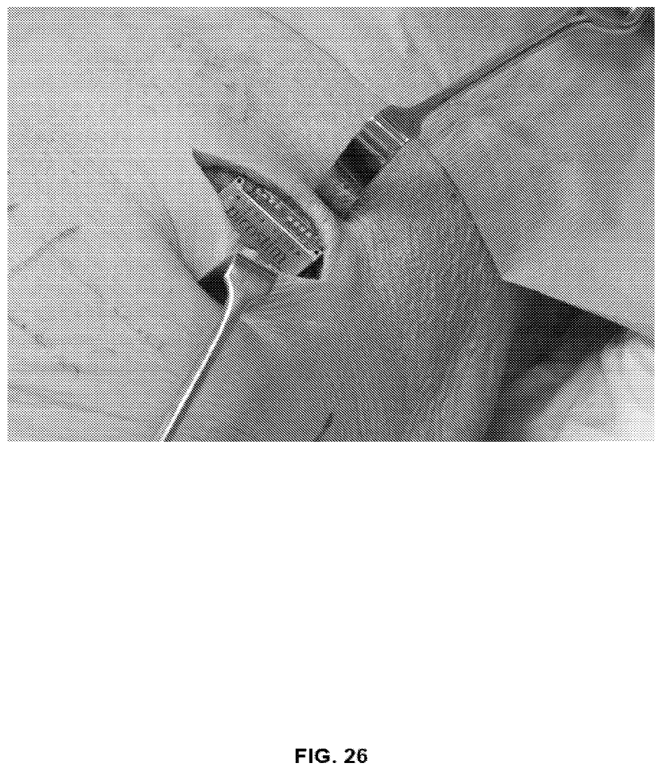

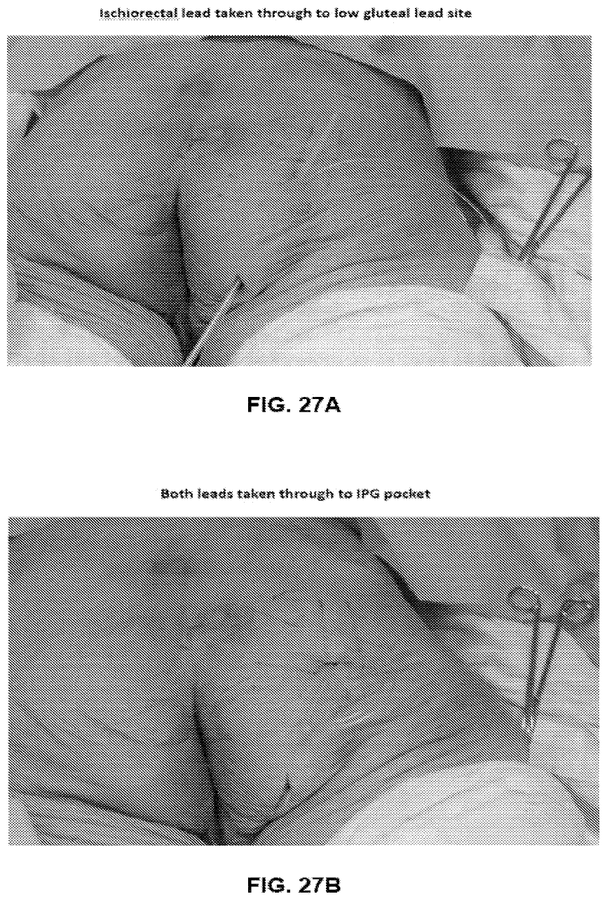

Provided herein are methods, devices, and systems for implanting at least one electrode lead and an implantable pulse generator (IPG) in an individual to treat incontinence. In some embodiments, a small pocket sized to hold an IPG is created in the ipsilateral upper buttock. In some embodiments, the pocket is created using a transverse skin incision. In some embodiments, the location of the pocket may be pre-marked prior to the implantation procedure while the individual is awake. In some embodiments, the location of the pocket may be chosen to avoid contacting clothing items, including but not limited to belts. FIG. 26 shows an exemplary embodiment of IPG pocket created in upper buttock. In some embodiments, the pocket is close to the skin surface to allow for recharging. In some embodiments, the pocket is no more than about 1 cm, 2 cm, 3 cm, 4 cm, or 5 cm from the skin surface. In some embodiments, the pocket is no more than 2 cm from the skin surface. In some embodiments, the pocket is of a sufficient size to comfortably accommodate the IPG without permitting excessive movement of the IPG within the pocket. In some embodiments, care is taken to separate the IPG from underlying gluteal muscle epimysium with a layer of fat in between the IPG and the epimysium.

### Electrode Lead Tunnelling

In some cases, the system described herein may be provided with a tunnelling tool (TT). In some cases, the tunnelling tool is used to direct the leads from their point of fixation to the IPG. FIGS. 27A and 27B show exemplary images of a sequence of tunnelling the leads, where FIG. 27A shows the ischiorectal lead taken through the low gluteal lead site and FIG. 27B shows both leads taken through to the IPG pocket. In some cases, the straw is slide over the obturator. In some cases, the obturator is a sharp metal obturator. In some cases, the assembly is bent to reflect the chosen tunnelling path. In some cases, the TT is pushed through the gluteal fat from the ischiorectal anchoring point to the low gluteal anchoring point while ensuring not to damage or displace the lead or the anchors. In some cases, the obturator is removed from the individual, leaving the straw in place. In some cases, the lead is threaded carefully through the straw and the straw is removed over the lead. In some cases, the process is repeated by taking both leads from the low gluteal point to the IPG pocket, ensuring not to damage or displace the lead or the anchors. In some cases, tunnelling the leads comprises using a system using standard surgical instruments. In some cases, tunnelling the leads comprises using a tunneller that connects at one end to a straw and drags the lead through the straw. In some cases, the surgeon takes care not to pass the tunnelling tool into the gluteal muscles and stays in subcutaneous fat to avoid bleeding, vascular injury, or nerve injury.

### Connecting Leads to IPG

In some cases, the electrode lead is connected to the IPG to deliver electrical stimulation to the tissue in contact with the electrodes of the lead. In some cases, before insertion into the IPG, the PNS leads are wiped clean and dry. In some cases, each PNS lead is inserted into the header of the IPG. In some cases, a visual check is performed to ensure that the leads have been placed correctly into the IPG. In some cases, the torque wrench is passed through the slit in the screw hole, which is located in the upper part of the IPG header. In some cases, the setscrew on the IPG is tightened until the torque wrench clicks once, where the click sound is an indicator that the setscrew is tightened. In some cases, to tighten the setscrew, the setscrew is rotated with the torque wrench clockwise until one click is heard. In some cases, to loosen the setscrew, rotate using the torque wrench anticlockwise. In some cases, the wrench is torque limiting as to prevent overtightening of the setscrew. In some cases, the supplied wrench only is used to tighten the set screw. In some cases, other tools may cause overtightening of the setscrew and damage to the PNS electrode.

### IPG Placement

In some embodiments, the IPG is placed in a certain orientation in the pocket. In some embodiments, the IPG is inserted with the engraved inscription “THIS SIDE OUT” facing the skin. In some embodiments, if the IPG faces the wrong way, the IPG may not charge. In some embodiments, if the IPG faces the wrong way, a revision surgery may be needed. In some embodiments, any excess length of the PNS lead is rolled up under the IPG to prevent lead injury during any revision surgery for the stimulator.

### Check Impedances

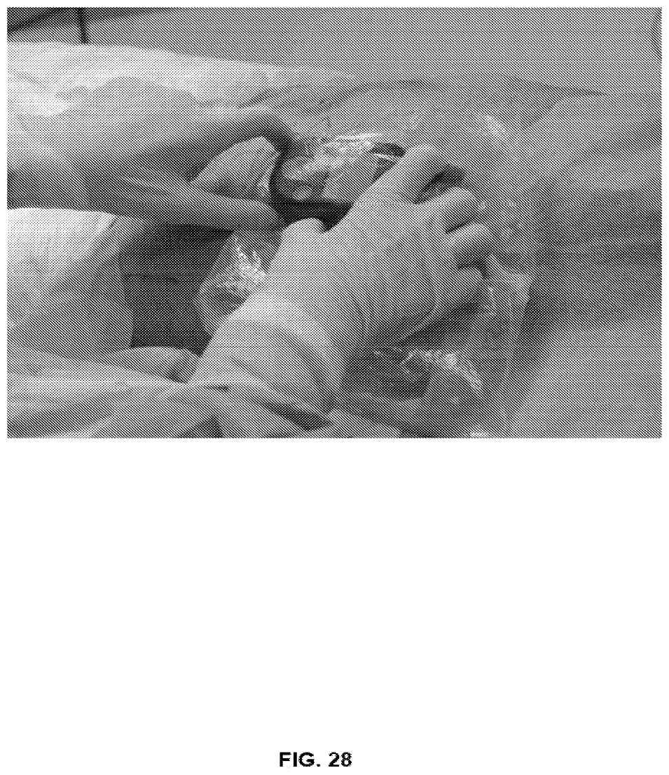

Often, a check of the impedance may be performed to verify the connection between the lead and the IPG. In some embodiments, an impedance check is performed using the Patient Controller or PicoPC software. In some embodiments, the Patient Controller nor the charging coil are sterile. In some embodiments, the Patient Controller or charging coil may be enclosed in a surgical sterile bag when checking impedances. FIG. 28 shows an exemplary image of the patient controller in a sterile bag to check impedance. Alternatively or in combination, IPG is linked to the Patient Controller prior to procedure and the link is maintained throughout the procedure. In some embodiments, if the impedance measurements are not satisfactory after a few attempts, the lead is removed from the header and re-inserted. In some embodiments, if this does not resolve the issue, the IPG or the PNS lead may be replaced. In some embodiments, the incision may be closed if the impedance measurements are satisfactory.

### Wound Closure

Usually, after the lead placement has been verified and the impedance measurements are satisfactory, the incision opening may be closed. In some embodiments, the fat is closed over the IPG with an interrupted absorbable suture. In some embodiments, the interrupted absorbable suture comprises as 2-0 vicryl. In some embodiments, the skin is closed with a continuous subcuticular absorbable monofilament suture. In some embodiments, skin glue is used over incision to seal the incision. In some embodiments, a dressing is placed over the incision.

### Intra-Operative Assessments

In some embodiments, non-invasive pelvic floor EMG may be recorded. In some embodiments, non-invasive pelvic floor EMG may be recorded using a trans-vaginal probe in women and transrectally in men. In some embodiments, a solid-state urodynamic catheter may be placed to measure pressure and urethral length. In some embodiments, a urethral transducer may be inserted in the individual to monitor and improve the accuracy of lead placement. In some embodiments, a transducer on urinary catheter may be used to monitor and improve the accuracy of lead placement. In some embodiments, various measurements may be taken intraoperatively. In some embodiments, intraoperatively measurements comprise one or more of pelvic floor ‘far field’ EMG (from electrode leads with focus on anterior lead); pelvic floor surface EMG (from transvaginal probe: women only); pudendal nerve ENG (from electrode leads), urethral sphincter pressure (from catheter mounted transducer); amplitude, frequency, and pulse width for each electrode on the lead; and surgeon test actuation of device by inertia (tapping).

Ambulatory Assessments after Procedure

In some embodiments, various ambulatory assessments may be taken determine the effectiveness of the procedure. In some embodiments, the implanted IPG permits telemetric downloading of data (inputs, outputs, and event classification). In some embodiments, with the participant in an awake ambulatory setting, a series of resting and provoked electrophysiological data may be recorded. In some embodiments, at treatment initiation (24-48 hours post-implant) sensory and motor responses is determined from the different electrodes on the implanted leads. In some embodiments, based upon the responses, the electrodes with the most adequate response may be selected to initiate treatment.

In some embodiments, the patients are subjected to different physiological events to program the IPG. In some embodiments, these events comprise one or more of: coughing, Valsalva maneuver, and picking up a 5 kg weight. In some embodiments, pelvic floor EMG with the Maple probe and urethral pressures are measured. In some embodiments, 1 hour continuous ‘resting’ recording of inputs and outputs (downloaded by telemetry) is taken. In some embodiments, recording during controlled participant provoked events, such as coughing, Valsalva, lifting 5 Kg weight, are obtained. In some embodiments, recording during pelvic floor surface EMG (from transvaginal probe: women only) to correlate inputs from lead vs. surface EMG are obtained. In some embodiments, patient tolerances of basal stimulation ramping and actuation parameters are obtained. In some embodiments, standard urodynamic tests are performed at 48 hours. In some embodiments, undesired detrusor contractions (UDCs) (with or without reporting of urge) are recorded during bladder filling to assess the acute effect of patient-actuation of device. In some embodiments, a standard 1-hour pad test is performed.

### Clinical Outcomes

In some embodiments, clinical outcomes may be assessed using a 5-day voiding diary recording number of voids, number of urgency episodes, number of leaks with severity of leaks to derive: stress and urge UI (summative) episodes per unit time; stress UI episodes per unit time; urge UI episodes per unit time; urgency to void episodes per unit time; total voiding frequency per unit time; responder rate: based on >50% decrease in UI episodes per unit time; functional cure rate defined as either >90% decrease in mean total UI episodes from baseline OR mean <1 UIE per week: and ICIQ-SF-UI questionnaire.

### DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of measurement and include determining if an element is present or not (for example, detection). These terms can include quantitative, qualitative, or quantitative and qualitative determinations. Assessing is alternatively relative or absolute. “Detecting the presence of” includes determining the amount of something present, as well as determining whether it is present or absent.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. The disease can be endometriosis. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An “ex vivo” assay is not performed on a subject. Rather, it is performed upon a sample separate from a subject. An example of an “ex vivo” assay performed on a sample is an “in vitro” assay.

The term “in vitro” is used to describe an event that takes places contained in a container for holding laboratory reagent such that it is separated from the living biological source organism from which the material is obtained. In vitro assays can encompass cell-based assays in which cells alive or dead are employed. In vitro assays can also encompass a cell-free assay in which no intact cells are employed.

As used herein, the term ‘about’ a number refers to that number plus or minus 10% of that number. The term ‘about’ a range refers to that range minus 10% of its lowest value and plus 10% of its greatest value.

As used herein, the terms “treatment” or “treating” are used in reference to an intervention regimen for obtaining beneficial or desired results in the recipient. Beneficial or desired results include but are not limited to a therapeutic benefit and/or a prophylactic benefit. A therapeutic benefit may refer to reduction, eradication, or amelioration of symptoms or of an underlying disorder being treated. Also, a therapeutic benefit can be achieved with the reduction, eradication, or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### EXAMPLES

The following examples are included for illustrative purposes only and are not intended to limit the scope of the invention.

### Example 1: Siting an Electrode Lead by Ischiorectal Approach

The pudendal nerve may be accessed in an individual by ischiorectal approach to place an electrode lead by the following steps:

Incise skin just medial to ischial tuberosity at the level determined above from AP measurements.

Open soft tissues to admit finger easily and allow clear palpation of ischial tuberosity and also flexibility on angulation of introducer.

With needle retracted, advance introducer toward marked ‘area of interest’ and to a depth as marked on the patient laterally. This can be followed fluoroscopically in AP view if required.

When introducer meets resistance, deploy internal needle using screw lock mechanism.

Advance about 5-10 mm further to perforate or pass close to the sacrotuberous ligament and then retract needle leaving the obturator in place.

Connect J-hook electrical connector to most distal contact point and advance introducer from bottom to top of ‘region of interest’ (approx. 2.5 cm) guided by X-ray and EMG measurement.

Stimulate all electrodes on the introducer while observing anal contraction and recording pelvic floor EMG using the intravaginal Maple device.

EMG and some evidence of anal contraction should be recorded at low stimulation amplitude at n−1 contact points, where n is total number of electrodes (document in operative record).

Remove obturator and introduce lead up to the marking point leaving the lead electrodes at the same position as the introducer ring electrodes.

Remove the introducer completely keeping lead stable at position using continuous fluoroscopy.

Verify adequate position of the lead by electrical stimulation and X-ray.

### Example 2: Siting an Electrode Lead by Low Gluteal Approach

The pudendal nerve may be accessed in an individual by low gluteal approach to place an electrode lead by the following steps:

Incise skin 2 cm just lateral from the inner brin of the ischial bone as determined by the prior AP measurements. Open soft tissues to admit finger easily to allow palpation of the bone and flexibility on angulation of introducer.

Probe the ‘sweet spot’ (where the nerve angulates into Alcock's canal). An oblique X-ray view may help to determine the angulation required to follow the nerve.

Start just below L2. With needle retracted, advance JEB introducer slightly downwards until resistance is felt, when hitting the bone. Angulate medially and insert the introducer 5 mm.

Connect J-hook electrical connector to most distal contact point and evaluate EMG responses when advancing introducer 5 mm (needle is retracted).

If no or inadequate EMG responses are measured, remove the introducer and repeat the procedure 5 mm below L2.

Continue until satisfactory EMG response is measured.

Further advance the introducer (with needle retracted and J hook connected), forwards and medial, following this fluoroscopically in AP view and under EMG measurement such that the introducer is seen to follow the curve formed by the inferior pubic ramus at the inferior border of the obturator foramen. The introducer will eventually point in the direction of the pubic symphysis.

Stimulate all 4 electrodes on the introducer-ideally pelvic floor EMG should be recorded at low stimulation amplitude at 3 contact points.

Record these data in the operative record.

Remove obturator and introduce lead up to the marking point leaving the lead electrodes at the same position as the introducer electrodes.

Remove the introducer completely keeping lead stable at position using continuous fluoroscopy.

Verify adequate position of the lead by electrical stimulation and X-ray.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### NUMBERED EMBODIMENTS

Numbered embodiment 1 comprises a device for placing an electrode lead to a target site, the device comprising: an introducer sheath comprising a sheath elongate shaft having a lumen and a sheath handle at a distal end of the sheath shaft, wherein the introducer sheath is configured to carry at least one electrode on an outer surface of the sheath elongate shaft; an obturator comprising an obturator elongate shaft having a lumen and an obturator handle at a distal end of the obturator elongate shaft, wherein the obturator elongate shaft is configured to fit inside the sheath lumen; and a needle comprising a needle elongate shaft having a lumen, a needle handle at a distal end of the needle elongate shaft, and a needle tip at the proximal end of the needle elongate shaft, wherein the needle elongate shaft is configured to fit inside the obturator lumen, and wherein the lumen of the needle elongate shaft is configured to allow the electrode lead to pass through; wherein the introducer sheath has a stiffness sufficient to guide the placement of the electrode lead at the target site, wherein the target site is a pudendal nerve.

Numbered embodiment 2 comprises the device of embodiment 1, wherein the obturator handle comprise a latch configured to attach to the sheath handle.

Numbered embodiment 3 comprises the device of embodiment 1 or 2, wherein the at least one electrode comprises a flexible printed circuit.

Numbered embodiment 4 comprises the device of embodiment 1 or any preceding embodiments, wherein the at least one electrode is wrapped around the outer surface of the sheath elongate shaft.

Numbered embodiment 5 comprises the device of embodiment 1 or any preceding embodiments, wherein the at least one electrode is bonded to the outer surface of the sheath elongate shaft.

Numbered embodiment 6 comprises the device of embodiment 1 or any preceding embodiments, wherein the needle tip is blunt and has a lumen.

Numbered embodiment 7 comprises the device of embodiment 6 or any preceding embodiments, wherein the needle tip is configured to protrude at least 1 mm beyond the end of the obturator lumen.

Numbered embodiment 8 comprises the device of embodiment 6 or any preceding embodiments, wherein the needle tip is configured to protrude by a movement of the needle handle.

Numbered embodiment 9 comprises the device of embodiment 1 or any preceding embodiments, wherein the needle tip is configured to be retractable into the obturator lumen.

Numbered embodiment 10 comprises the device of embodiment 1 or any preceding embodiments, wherein the needle tip has an angle ranging from about 15 degrees to about 45 degrees from the needle elongate shaft.

Numbered embodiment 11 comprises the device of embodiment 10 or any preceding embodiments, wherein the needle tip angle is configured to advance the needle tip through tissue.

Numbered embodiment 12 comprises the device of embodiment 1 or any preceding embodiments, wherein a proximal end of the sheath elongate shaft is angled to form an angle of about 30 degrees to about 90 degrees.

Numbered embodiment 13 comprises the device of embodiment 12 or any preceding embodiments, wherein the angle of the proximal end of the sheath elongate shaft allows for advancing the device with little to no damage to surrounding tissue.

Numbered embodiment 14 comprises the device of embodiment 1 or any preceding embodiments, wherein the needle has a diameter of about 0.4 mm to about 2 mm.

Numbered embodiment 15 comprises the device of embodiment 1 or any preceding embodiments, wherein the needle has a diameter of between 12 and 26 gauge.

Numbered embodiment 16 comprises the device of embodiment 1 or any preceding embodiments, wherein the obturator has a diameter of about 1 mm to about 4 mm.

Numbered embodiment 17 comprises the device of embodiment 1 or any preceding embodiments, wherein the introducer sheath has a diameter of about 1 mm to about 5 mm.

Numbered embodiment 18 comprises the device of embodiment 1 or any preceding embodiments, wherein the sheath elongate shaft comprises high density polyethylene (HDPE).

Numbered embodiment 19 comprises the device of embodiment 1 or any preceding embodiments, wherein the obturator elongate shaft comprises stainless steel.

Numbered embodiment 20 comprises the device of embodiment 1 or any preceding embodiments, wherein the needle elongate shaft comprises stainless steel.

Numbered embodiment 21 comprises the device of embodiment 1 or any preceding embodiments, wherein the introducer sheath has a Young's modulus of about 10 mega pascal (MPa) to about 10,000 MPa.

Numbered embodiment 22 comprises the device of embodiment 1 or any preceding embodiments, wherein the sheath elongate shaft has a Young's modulus of about 10 mega pascal (MPa) to about 10,000 MPa.

Numbered embodiment 23 comprises the device of embodiment 1 or any preceding embodiments, wherein the introducer sheath and the needle have a combined Young's modulus sufficient to allow for the device to penetrate a deep surgical plane in an individual.

Numbered embodiment 24 comprises the device of embodiment 23 or any preceding embodiments, wherein the deep surgical plane comprises a surgical plane of muscle, fat, or any combination thereof.

Numbered embodiment 25 comprises the device of embodiment 1 or any preceding embodiments, wherein the introducer sheath and needle have a combined Young's modulus to sufficient allows a user to place the lead adjacent to the pudendal nerve.

Numbered embodiment 26 comprises a method for accessing a pudendal nerve in a subject, the method comprising: (a) using needle electrodes to determine optimal site of introducer insertion including laterality of response; (b) creating an opening on a skin surface medial to ischial tuberosity: (c) advancing an electrode introducer through the opening to a predetermined depth; (d) deploying and advancing a needle tip of the electrode introducer to pass close to or penetrate ligamentous structures; and (e) accessing the pudendal nerve.

Numbered embodiment 27 comprises the method of embodiment 26 wherein the method further comprising placing at least one electrode to the pudendal nerve, wherein the electrode introducer is configured to hold and deliver the electrode.

Numbered embodiment 28 comprises the method of embodiment 27 or any preceding embodiments, wherein the at least one electrode is placed at the pudendal nerve trunk proximal to Alcock's canal.

Numbered embodiment 29 comprises the method of embodiment 26 or any preceding embodiments, the method further comprising identifying and providing on a skin surface a plurality of surface markings corresponding a plurality of anatomical locations based an image of the plurality of anatomical locations, wherein the plurality of surface markings is used to determine the location of the opening on the skin surface.

Numbered embodiment 30 comprises the method of embodiment 29 or any preceding embodiments, wherein the plurality of anatomical locations comprises one or more of medial border of ischial bone, lateral border of ischial bone, superior border of inferior pubic ramus, inferior border of inferior pubic ramus, inferior border of acetabulum, and superior border of greater trochanter of femur.

Numbered embodiment 31 comprises the method of embodiment 29 or any preceding embodiments, wherein the image is obtained by fluoroscopy.

Numbered embodiment 32 comprises the method of embodiment 29 or any preceding embodiments, wherein the plurality of surface markings comprise one or more of a horizontal line through the superior tip of the greater trochanter of the femur (L3), a horizontal line through the inferior border of the acetabulum (L2), a curved line on the inner surface of the ischial bone (L1), and a marking representing the ischial spine that crosses with L3 line.

Numbered embodiment 33 comprises the method of embodiment 32 or any preceding embodiments, wherein the pudendal nerve trunk is located in an area medial to the L1 curved line and substantially superior to the L2 line.

Numbered embodiment 34 comprises the method of embodiment 32 or any preceding embodiments, wherein a distal portion of the pudendal nerve is located in an area medial to the L1 curved line and substantially inferior to L2 line.

Numbered embodiment 35 comprises the method of embodiment 26 or any preceding embodiments, wherein the electrode introducer is advanced toward the ischial spine.

Numbered embodiment 36 comprises the method of embodiment 27 or any preceding embodiments, the method further comprising retracting the needle tip into the electrode introducer prior to placing the at least one electrode at the pudendal nerve.

Numbered embodiment 37 comprises the method of embodiment 26 or any preceding embodiments, wherein advancing the electrode introducer is guided by imaging.

Numbered embodiment 38 comprises the method of embodiment 37 or any preceding embodiments, wherein the imaging comprises fluoroscopy.

Numbered embodiment 39 comprises the method of embodiment 27 or any preceding embodiments, the method further comprising stimulating at least one electrode on the pudendal nerve at a low stimulation amplitude.

Numbered embodiment 40 comprises the method of embodiment 39 or any preceding embodiments, the method further comprising observing contraction of the anal sphincter or a urethral pressure contraction or a combination thereof.

Numbered embodiment 41 comprises the method of embodiment 40 or any preceding embodiments, the method further comprising obtaining an electromyography (EMG) reading of a pelvic floor muscle.

Numbered embodiment 42 comprises the method of embodiment 41 or any preceding embodiments, the method further comprising obtaining a transvaginal electromyography (EMG) reading.

Numbered embodiment 43 comprises the method of embodiment 42 or any preceding embodiments, wherein the placement of the at least one electrode is adjusted based on the EMG reading.

Numbered embodiment 44 comprises the method of embodiment 27 or any preceding embodiments, the method further comprising verifying the placement of the at least one electrode by electrical stimulation.

Numbered embodiment 45 comprises the method of embodiment 41 or any preceding embodiments, the method further comprising verifying the placement of the at least one electrode by EMG of the anal sphincter and the pelvic floor.

Numbered embodiment 46 comprises the method of embodiment 27 or any preceding embodiments, the method further comprising verifying the placement of the at least one electrode by X-ray imaging.

Numbered embodiment 47 comprises the method of embodiment 26 or any preceding embodiments, the method further comprising removing the electrode introducer through the opening after placing the at least one electrode at the pudendal nerve.

Numbered embodiment 48 comprises the method of embodiment 47 or any preceding embodiments, the method comprising a change in conformity of the electrode lead tip after removal of the electrode introducer with the purpose of preventing lead movement.

Numbered embodiment 49 comprises the method of embodiment 26 or any preceding embodiments, wherein the subject has experienced or is at risk for experiencing an episode of incontinence.

Numbered embodiment 50 comprises a method for accessing a pudendal nerve in a subject, the method comprising: (a) using needle electrodes to determine optimal site of introducer insertion including laterality of response; (b) creating an opening on a skin surface at a predetermined incision distance laterally from inner brim of ischial bone; (c) advancing an electrode introducer through the opening down to the ischial bone and along the medial border of the ischial bone; (d) moving the electrode introducer into a gap between sacrotuberous ligament and sacrospinous ligament; (e) further advancing the electrode introducer following a curve of the inferior pubic ramus at inferior border of obturator foramen and in the direction of the pubic symphysis; and (f) accessing the pudendal nerve.

Numbered embodiment 51 comprises the method of embodiment 50, wherein the method further comprising placing at least one electrode at the pudendal nerve, wherein the electrode introducer is configured to hold and deliver the at least one electrode.

Numbered embodiment 52 comprises the method of embodiment 51 or any preceding embodiments, wherein the at least one electrode is placed at an anterior portion of the pudendal nerve or branches thereof.

Numbered embodiment 53 comprises the method of embodiment 52 or any preceding embodiments, wherein the anterior branch of the pudendal nerve comprises the dorsal genital nerve.

Numbered embodiment 54 comprises the method of embodiment 51 or any preceding embodiments, the method further comprising deploying a needle tip of the at least one electrode introducer in step (b).

Numbered embodiment 55 comprises the method of embodiment 54 or any preceding embodiments, the method further comprising retracting the needle tip in step (c) and prior to placing the at least one electrode at the pudendal nerve.

Numbered embodiment 56 comprises the method of embodiment 50 or any preceding embodiments, the method further comprising advancing the electrode introducer anteriorly in the ischiorectal fossa and below the pelvic floor in step (d).

Numbered embodiment 57 comprises the method of embodiment 50 or any preceding embodiments, wherein the method further comprising identifying and providing on a skin surface a plurality of surface markings corresponding a plurality of anatomical locations based an image of the plurality of anatomical locations, wherein the plurality of surface markings is used to determine the location of the opening on the skin surface.

Numbered embodiment 58 comprises the method of embodiment 57 or any preceding embodiments, wherein the plurality of anatomical locations comprises one or more of medial border of ischial bone, lateral border of ischial bone, superior border of inferior pubic ramus, inferior border of inferior pubic ramus, inferior border of acetabulum, and superior border of greater trochanter of femur.

Numbered embodiment 59 comprises the method of embodiment 57 or any preceding embodiments, wherein the image is obtained by fluoroscopy.

Numbered embodiment 60 comprises the method of embodiment 57 or any preceding embodiments, wherein the plurality of surface markings comprise one or more of a horizontal line through the superior tip of the greater trochanter of the femur (L3), a horizontal line through the inferior border of the acetabulum (L2), a curved line on the inner surface of the ischial bone (L1), and a marking representing the ischial spine that crosses with L3 line.

Numbered embodiment 61 comprises the method of embodiment 60 or any preceding embodiments, wherein the pudendal nerve trunk is located in an area medial to the L1 curved line and substantially superior to the L2 line.

Numbered embodiment 62 comprises the method of embodiment 60 or any preceding embodiments, wherein a distal portion of the pudendal nerve is located in an area medial to the L1 curved line and substantially inferior to L2 line.

Numbered embodiment 63 comprises the method of embodiment 50 or any preceding embodiments, wherein advancing the electrode introducer is guided by imaging.

Numbered embodiment 64 comprises the method of embodiment 63 or any preceding embodiments, wherein the imaging comprises fluoroscopy.

Numbered embodiment 65 comprises the method of embodiment 50 or any preceding embodiments, wherein advancing the electrode lead is guided by the position of the already sited pudendal trunk lead.

Numbered embodiment 66 comprises the method of embodiment 51 or any preceding embodiments, wherein the method further comprising stimulating the at least one electrode on the pudendal nerve at a low stimulation amplitude.

Numbered embodiment 67 comprises the method of embodiment 66 or any preceding embodiments, wherein the method further comprising observing contraction of the anal or urethral sphincter.

Numbered embodiment 68 comprises the method of embodiment 67 or any preceding embodiments, wherein an electromyography (EMG) reading of the contraction of a urethral or anal sphincter is taken at least one contact point.

Numbered embodiment 69 comprises the method of embodiment 68 or any preceding embodiments, wherein the method further comprising obtaining an electromyography (EMG) reading of a pelvic floor muscle.

Numbered embodiment 70 comprises the method of embodiment 69 or any preceding embodiments, the method further comprising obtaining a transvaginal electromyography (EMG) reading.

Numbered embodiment 71 comprises the method of embodiment 70 or any preceding embodiments, wherein the EMG reading is taken at least one contact point.

Numbered embodiment 72 comprises the method of embodiment 51 or any preceding embodiments, wherein the method further comprising verifying the placement of the at least one electrode by electrical stimulation.

Numbered embodiment 73 comprises the method of embodiment 512 or any preceding embodiments, wherein the method further comprising verifying the placement of the at least one electrode by EMG of the anal sphincter and the pelvic floor.

Numbered embodiment 74 comprises the method of embodiment 51 or any preceding embodiments, the method further comprising verifying the placement of the at least one electrode by X-ray imaging.

Numbered embodiment 75 comprises the method of embodiment 51 or any preceding embodiments, wherein the method further comprising removing the electrode introducer through the opening after placing the at least one electrode at the pudendal nerve.

Numbered embodiment 76 comprises the method of embodiment 50 or any preceding embodiments, wherein the method comprising a change in conformity of the electrode lead tip after removal of the electrode introducer with the purpose of preventing lead movement.

Numbered embodiment 77 comprises the method of embodiment 51 or any preceding embodiments, wherein the subject has experienced or is at risk for experiencing an episode of incontinence.

1.-34. (canceled)

35. A method for securing an electrode lead to a tissue near a pudendal nerve of a patient, the method comprising:
inserting a needle toward an ischial spine;
applying stimulation through the needle;
locating the pudendal nerve by measuring an EMG response to the stimulation applied through the needle;
after locating the pudendal nerve, advancing an introducer along a path of the needle;
advancing the electrode lead through the introducer; and
anchoring the electrode lead into the tissue surrounding the pudendal nerve.

36. The method of claim 35, further comprising adjusting a location of the introducer based on a pelvic floor EMG response.

37. The method of claim 35, further comprising adjusting a location of the introducer based on an anal sphincter EMG response.

38. The method of claim 35, further comprising applying stimulation through the electrode lead to confirm a position of the electrode lead in the tissue.

39. The method of claim 38, further comprising adjusting the position of the electrode lead based on an EMG response to the stimulation applied through the electrode lead.

40. The method of claim 39, wherein the EMG response is measured at a pelvic floor.

41. The method of claim 39, wherein the EMG response is measured at an anal sphincter.

42. The method of claim 35, further comprising marking a buttocks to indicate a greater trochanter and ischial tuberosity to locate a surface position of the ischial spine.

43. The method of claim 35, further comprising locating the pudendal nerve by measuring a urethral pressure indicative of urethral contraction.

44. The method of claim 35, further comprising inserting the needle by ischiorectal approach.

45. The method of claim 35, further comprising inserting the needle by a posterior or gluteal approach.

46. The method of claim 35, further comprising placing the electrode lead on an anterior branch of the pudendal nerve.

47. The method of claim 35, further comprising fixing the electrode lead to the tissue using one or more fins.

48. The method of claim 35, further comprising implanting an implantable pulse generator.

49. The method of claim 48, further comprising tunnelling to direct the electrode lead to an implanted pulse generator.

50. A method for securing an electrode lead to a tissue near a pudendal nerve of a patient, the method comprising:
advancing an introducer toward the pudendal nerve;
advancing the electrode lead through the introducer;
applying stimulation through the electrode lead to confirm a position of the electrode lead in the tissue;
adjusting the position of the electrode lead based on an EMG response to the stimulation applied through the electrode lead; and
anchoring an electrode lead into the tissue surrounding the pudendal nerve.

51. The method of claim 50, further comprising locating the pudendal nerve by measuring a urethral pressure indicative of urethral contraction.

52. The method of claim 50, further comprising adjusting a location of the introducer based on a pelvic floor EMG response.

53. The method of claim 50, further comprising adjusting a location of the introducer based on an anal sphincter EMG response.

54. The method of claim 50, wherein the EMG response is measured at a pelvic floor.

55. The method of claim 50, wherein the EMG response is measured at an anal sphincter.

56. The method of claim 50, further comprising fixing the electrode lead to the tissue using one or more fins.
